(19) United States
(12) Patent Application Publication
(10) Pub. No.: US 2014/0296129 A1
(43) Pub. Date: Oct. 2, 2014

(54) REGULATION OF RECEPTOR EXPRESSION THROUGH DELIVERY OF ARTIFICIAL TRANSCRIPTION FACTORS

(71) Applicant: ALIOPHTHA AG, Basel (CH)

(72) Inventors: Josef Flammer, Binningen (CH); Albert Neutzner, Schliengen (DE); Alice Huxley, Binningen (CH)

(21) Appl. No.: 14/349,443

(22) PCT Filed: Oct. 10, 2012
(86) PCT No.: PCT/EP2012/069981

§ 371 Date: Apr. 3, 2014

(30) Foreign Application Priority Data
Oct. 11, 2011 (EP) 11184706.7

(51) Int. Cl. C07K 14/47

(52) U.S. Cl. CPC C07K 14/47
USPC 514/1.1; 530/350

(57) ABSTRACT

The invention relates to an artificial transcription factor comprising a polydactyl zinc finger protein targeting specifically a receptor gene promoter fused to an inhibitory or activatory protein domain, a nuclear localization sequence, and a protein transduction domain. In particular examples these receptor gene promoters regulate the expression of the endothelin receptor A, the endothelin receptor B, the Toll-like receptor 4 or the high-affinity IgE receptor. Artificial transcription factors directed to the endothelin A or B receptors are useful in the treatment of diseases modulated by endothelin, such as cardiovascular diseases, and, in particular, eye diseases, e.g. retinal vein occlusion, retinal artery occlusion, macular edema, optic neuropathy, central serous chorioretinopathy, retinitis pigmentosa, Leber's hereditary optic neuropathy, and the like. Artificial transcription factors directed to the Toll-like receptor 4 or the IgE receptor are useful for the treatment of autoimmune disorders, and the like, and allergic disorders, respectively.

### FIELD OF THE INVENTION

The invention relates to artificial transcription factors comprising a polydactyl zinc finger protein targeting specifically a receptor gene promoter fused to an inhibitory or activatory domain, a nuclear localization sequence, and a protein transduction domain, and their use in treating diseases modulated by the binding of specific effectors to such receptors.

### BACKGROUND ART

Artificial transcription factors (ATFs) are proposed to be useful tools for modulating gene expression (Sera T., 2009, Adv Drug Deliv Rev 61, 513-526). Many naturally occurring transcription factors, influencing expression either through repression or activation of gene transcription, possess complex specific domains for the recognition of a certain DNA sequence. This makes them unattractive targets for manipulation if one intends to modify their specificity and target gene(s). However, a certain class of transcription factors contains several so called zinc finger (ZF) domains, which are modular and therefore lend themselves to genetic engineering. Zinc fingers are short (30 amino acids) DNA binding motifs targeting almost independently three DNA base pairs. A protein containing several such zinc fingers is thus able to recognize longer DNA sequences. A hexameric zinc finger protein (ZFP) recognizes an 18 base pairs (bp) DNA target, which is almost unique in the entire human genome. Initially thought to be completely context independent, more in-depth analyses revealed some context specificity for zinc fingers (Klug A., 2010, Annu Rev Biochem 79, 213-231). Mutating certain amino acids in the zinc finger recognition surface altering the binding specificity of ZF modules resulted in defined ZF building blocks for most of 5′-GNN-3′,5′-CNN-3′,5′-ANN-3′, and some 5′-TNN-3′ codons (e.g. so-called Barbas modules, see Dreier B., Barbas C. F. 3^{rd} et al., 2005, J Biol Chem 280, 35588-35597). While early work on artificial transcription factors concentrated on a rational design based on combining preselected zinc fingers with a known 3 bp target sequence, the realization of a certain context specificity of zinc fingers necessitated the generation of large zinc finger libraries which are interrogated using sophisticated methods such as bacterial or yeast one hybrid, phage display, compartmentalized ribosome display or in vivo selection using FACS analysis.

Using such artificial zinc finger proteins, DNA loci within the human genome can be targeted with high specificity. Thus, these zinc finger proteins are ideal tools to transport protein domains with transcription-modulatory activity to specific promoter sequences resulting in the modulation of expression of a gene of interest. Suitable domains for the silencing of transcription are the Krueppel-associated domain (KRAB) as N-Terminal (SEQ ID NO: 1) or C-terminal (SEQ ID NO: 2) KRAB domain, the Sin3-interacting domain (SID, SEQ ID NO: 3) and the ERF repressor domain (ERD, SEQ ID NO: 4), while activation of gene transcription is achieved through herpes virus simplex VP16 (SEQ ID NO: 5) or VP64 (tetrameric repeat of VP16, SEQ ID NO: 6) domains (Beerli R. R. et al., 1998, Proc Natl Acad Sci USA 95, 14628-14633). In addition, transcriptionally active domains of proteins defined by gene ontology GO:0001071 (http://amigo.geneontology.org/cgi-bin/amigo/term_details?term=GO:0001071) are considered to achieve transcriptional regulation of target proteins.

A large percentage of all known drug targets are receptor molecules that are either stimulated or blocked by the action of small molecule drugs with oftentimes considerable off-target activities. Examples for such receptors are the histamine H1 receptor or alpha- and beta-adrenoreceptors, but in general proteins defined by gene ontology GO:0004888 and GO:0004930.

While small molecule drugs are not always able to selectively target a certain member of a given protein family due to the high conservation of specific features, biologicals offer great specificity as shown for antibody-based novel drugs. However, virtually all biologicals to date act extracellularly.

Especially above mentioned artificial transcription factors would be suitable to influence gene transcription in a therapeutically useful way. However, the delivery of such factors to the site of action—the nucleus—is not easily achieved, thus hampering the usefulness of therapeutic artificial transcription factor approaches, e.g. by relaying on retroviral delivery with all the drawbacks of this method such as immunogenicity and the potential for cellular transformation (Lund C. V. et al., 2005, Mol Cell Biol 25, 9082-9091).

So called protein transduction domains (PTDs) were shown to promote protein translocation across the plasma membrane into the cytosol/nucleoplasm. Short peptides such as the HIV derived TAT peptide (SEQ ID NO: 7) and others were shown to induce a cell-type independent macropinocytotic uptake of cargo proteins (Wadia J. S. et al., 2004, Nat Med 10, 310-315). Upon arrival in the cytosol, such fusion proteins were shown to have biological activity. Interestingly, even misfolded proteins can become functional following protein transduction most likely through the action of intracellular chaperones.

The vasoactive endothelin system plays an important role in the pathogenesis of various diseases. Endothelins, on the one hand, are involved in the regulation of blood supply and, on the other hand, are main players in the cascade of events induced by hypoxia. Endothelin is e.g. involved in the breakdown of the blood-brain or the blood-retina barrier and in the neovascularisation. Endothelin is furthermore involved in neurodegeneration but also the regulation of the threshold of pain sensation or even thirst feeling. Endothelin is also involved in regulation of intraocular pressure.

The action of endothelin is mediated by its cognate receptors, mainly endothelin receptor A, usually located on smooth muscle cells surrounding blood vessels. Influencing the endothelin system—systemically or locally—is of interest for the treatment of many diseases such as subarachnoidal or brain hemorrhages. Endothelin also influences the course of multiple sclerosis. Endothelin contributes to (pulmonary) hypertension, but also to arterial hypotension, cardiomyopathy and to Raynaud syndrome, variant angina and other cardiovascular diseases. Endothelin is involved in diabetic nephropathy and diabetic retinopathy. In the eye it further plays a role for the glaucomatous neurodegeneration, retinal vein occlusion, giant cell arthritis, retinitis pigmentosa, age related macula degeneration, central serous chorioretinopathy, Morbus Leber, Susac syndrome, intraocular hemorrhages, epiretinal gliosis and certain other pathological conditions.

The eye is an exquisite organ that strongly relies on a balanced and sufficient perfusion to meet its high oxygen demand. Failure to provide sufficient and stable oxygen supply causes ischemia-reperfusion injury leading to glial activation and neuronal damage as observed in glaucoma patients with progressing disease despite normal or normalized intraocular pressure. Insufficient blood supply also leads to hypoxia causing run-away neovascularization with the potential of further retinal damage as evident during diabetic retinopathy or wet age related macula degeneration. Eye tissue perfusion is under complex control and depends on blood pressure, intraocular pressure as well as local factors modulating vessel diameter. Such local factors are for example the mentioned endothelins, short peptides with a strong vasoconstrictive activity. Three isoforms of endothelins (ET-1, ET-2, and ET-3) are produced by endothelin converting enzyme from precursor molecules secreted by endothelial cells localized in the blood vessel wall. Two cognate receptors for mature ET are known, ETRA and ETRB. While ETRA is localized to smooth muscle cells forming vessels walls and promoting vasoconstriction, ETRB is mainly expressed on endothelial cells and acts vasodilatatory by promoting the release of nitric oxide, thus causing smooth muscle relaxation. ETRA and ETRB belong to the large class of G-protein coupled seven transmembrane helix receptors. The binding of ET to ETRA or ETRB results in G protein activation, thus triggering an increase in intracellular calcium concentration and thereby causing a wide array of cellular reactions.

Influencing the ET system pharmacologically might prove useful in cases where ET levels are elevated and ETs act in a detrimental fashion, such as during retinal vein occlusion, glaucomatous neurodegeneration, retinitis pigmentosa, giant cell arthritis, central serous chorioretinopathy, multiple sclerosis, optic neuritis, rheumatoid arthritis, Susac syndrome, radiation retinopathy, epiretinal gliosis, fibromyalgia and diabetic retinopathy. To this end, down-regulation of ETRA will aid to modulate disease outcome. But under certain circumstances, upregulation of ETRA and therefore an increased sensitivity towards ET might be desirable, for example to promote corneal wound healing during the recovery from corneal trauma or corneal ulcer.

In addition, ETRB-mediated signaling is connected to pathophysiological processes e.g. during cancer stem cell maintenance and tumor growth. In addition, upregulation of ETRB is associated with glaucomatous neurodegeneration while inhibition of ETRB was shown to act neuroprotective during glaucoma. Furthermore, ETRB is upregulated during inflammation. Thus, pharmacological modulation of ETRB through a specific artificial transcription factor will be useful in the treatment of cancer, the prevention of neurodegeneration and the modulation of inflammatory processes.

Bacterial cell wall components such as lipopolysaccharide (LPS) play important roles in the pathogenesis of various diseases. The presence of LPS in the body points to a bacterial infection that needs to be addressed by the immune system. Since LPS is a general component of gram negative bacteria, LPS constitutes a so called danger signal that can activate the immune system. LPS is recognized by the Toll-like receptor 4 (TLR4), a member of the larger family of Toll-like receptors involved in the recognition of varied danger signals or pathogen associated molecular patterns (PAMPs) associated with bacterial or viral infections. While recognition of LPS as danger signal is an important part of innate immunity, overstimulation or prolonged stimulation of the TLR4 receptor is connected to a variety of pathological conditions associated with chronic inflammation. Examples are various liver diseases such as alcoholic liver disease, nonalcoholic fatty liver disease, nonalcoholic steatohepatitis, chronic hepatitis B or C virus (HCV) infection, and HIV-HCV co-infection. Other diseases associated with TLR4 signaling are rheumatoid arthritis, artherosclerosis, psoriasis, Crohn's disease, uveitis, contact lens associated keratitis and corneal inflammation. In addition, TLR4-mediated signaling is involved in cancer progression and resistance to chemotherapy.

Influencing LPS recognition and TLR4 signaling pharmacologically might prove useful for diseases associated with chronic inflammation due to inappropriate activation of TLR4. Thus, downregulation of TLR4 protein through the action of a specific negative-regulatory artificial transcription factor targeted to the TLR4 promoter will aid to modulate disease outcome through breaking the vicious cycle of chronic inflammation caused by LPS.

Immunoglobulins isotype E (IgE) are part of the adaptive immune system and as such involved in the protection against infections but also neoplastic transformation. IgE is bound by the high-affinity IgE receptor (FCER1) localized on mast cells and basophiles. Binding of IgE to FCER1 followed by cross-linking these complexes via specific antigens called allergens leads to the release of various factors from mast cells and basophils causing the allergic response. Among these factors are histamine, leukotrienes, various cytokines but also lysozyme, tryptase or β-hexosaminidase. The release of these factors is associated with allergic diseases such as allergic rhinitis, asthma, eczema and anaphylaxis.

### SUMMARY OF THE INVENTION

The invention relates to an artificial transcription factor comprising a polydactyl zinc finger protein targeting specifically a receptor gene promoter fused to an inhibitory or activatory protein domain, a nuclear localization sequence, and a protein transduction domain, and to pharmaceutical compositions comprising such an artificial transcription factor. Furthermore the invention relates to the use of such artificial transcription factors for modulating the reaction of cells to external stimuli and to other soluble signaling molecules, and in treating diseases modulated by the binding of specific effectors to such receptors.

In a particular embodiment, the receptor gene promoter is the endothelin receptor A promoter. In another particular embodiment the invention relates to such an artificial transcription factor for use in influencing the cellular response to endothelin, for lowering or increasing endothelin receptor levels, and for use in the treatment of diseases modulated by endothelin, in particular for use in the treatment of such eye diseases.

Likewise the invention relates to a method of treating a disease modulated by endothelin comprising administering a therapeutically effective amount of an artificial transcription factor of the invention to a patient in need thereof.

In another particular embodiment the invention relates to an artificial transcription factor intermediate comprising a polydactyl zinc finger protein targeting specifically the endothelin receptor A promoter fused to an inhibitory or activatory protein domain and a nuclear localization sequence.

In another particular embodiment, the receptor gene promoter is the endothelin receptor B promoter. In another particular embodiment the invention relates to such an artificial transcription factor for use in influencing the cellular response to endothelin, for lowering or increasing endothelin receptor B levels, and for use in the treatment of diseases modulated by endothelin, in particular for use in the treatment of such eye diseases. Likewise the invention relates to a method of treating a disease modulated by endothelin comprising administering a therapeutically effective amount of an artificial transcription factor of the invention to a patient in need thereof.

In another particular embodiment the invention relates to an artificial transcription factor intermediate comprising a polydactyl zinc finger protein targeting specifically the endothelin receptor B promoter fused to an inhibitory or activatory protein domain and a nuclear localization sequence.

In another particular embodiment, the receptor gene promoter is the Toll-like receptor 4 promoter. In another particular embodiment the invention relates to such an artificial transcription factor for use in influencing the cellular response to lipopolysaccharide, for lowering or increasing Toll-like receptor 4 levels, and for use in the treatment of diseases modulated by lipopolysaccharide, in particular for use in the treatment of eye diseases. Likewise the invention relates to a method of treating a disease modulated by lipopolysaccharide comprising administering a therapeutically effective amount of an artificial transcription factor of the invention to a patient in need thereof.

In another particular embodiment the invention relates to an artificial transcription factor intermediate comprising a polydactyl zinc finger protein targeting specifically the Toll-like receptor 4 promoter fused to an inhibitory or activatory protein domain and a nuclear localization sequence.

In another particular embodiment, the receptor gene promoter is the high-affinity immunoglobulin epsilon receptor subunit alpha promoter. In another particular embodiment the invention relates to such an artificial transcription factor for use in influencing the cellular response to immunoglobulin E (IgE), for lowering or increasing high-affinity IgE receptor levels, and for use in the treatment of diseases modulated by IgE, in particular for use in the treatment of eye diseases. Likewise the invention relates to a method of treating a disease modulated by IgE comprising administering a therapeutically effective amount of an artificial transcription factor of the invention to a patient in need thereof.

In another particular embodiment the invention relates to an artificial transcription factor intermediate comprising a polydactyl zinc finger protein targeting specifically the high-affinity immunoglobulin epsilon receptor subunit alpha promoter fused to an inhibitory or activatory protein domain and a nuclear localization sequence.

### BRIEF DESCRIPTION OF THE FIGURES

FIG. 1: Altering Cellular Sensitivity by Regulating Receptor Expression

An artificial transcription factor containing a hexameric zinc finger (ZF) protein targeting specifically a receptor gene (RG) promoter (P) fused to an inhibitory/activatory domain (RD=regulatory domain) as well as a nuclear localization sequence (NLS) is transported into cells by the action of a protein transduction domain (PTD) such as TAT or others. Depending on the transcription-regulatory domain, receptor gene expression is either increased (+) or suppressed (−) resulting in an enhanced or diminished cellular sensitivity towards receptor (R1, R2 or R3) agonist (A), respectively.

FIG. 2: Human Endothelin Receptor A (ETRA) Promoter Region

Shown is the 5′ untranslated region of the ETRA gene containing the putative ETRA promoter. Highlighted are the transcription start (marked with +1) and potential 15 bp and 18 bp target sites (TS) for artificial transcription factors (underlined and marked with TS−855, TS−555, TS−487, TS−447, TS−306, TS−230, TS−103, TS−37, TS+74).

FIG. 3: Human Toll-Like Receptor 4 (TLR4) Promoter Region

The 5′ region of the TLR4 gene containing the TLR4 promoter is shown. Highlighted are the transcription start (marked with +1), the initiation codon and the open reading frame of the first exon (bold letters) and potential 18 bp target sites for specific artificial transcription factors (underlined and marked with TS−276, TS−55, TS+113).

FIG. 4: Human High-Affinity IgE Receptor A (FCER1A) Promoter Region

The 5′ region of the FCER1A gene containing the proximal, constitutive promoter is shown. Highlighted are the transcription start (marked with +1), the initiation codon and the open reading frame of the first exon (bold letters) and potential 18 bp target sites for specific artificial transcription factors (underlined and marked with TS−147 and TS+17).

FIG. 5: Human Endothelin Receptor B (ETRB) Promoter Region

The 5′ region of the ETRB gene containing the ETRB promoter is shown. Highlighted are the translation start (marked with +1) and potential 18 bp target sites for specific artificial transcription factors (underlined and marked with TS−1149 and TS−487). Since several, alternative transcription start sites are reported (Arai H. et al., 1993, J Biol Chem 268, 3463-70; Tsutsumi M. et al., 1999, Gene 4, 43-9) the translation start site was chosen as reference point for naming target sites.

FIG. 6: Artificial Transcription Factors

A) Various zinc finger proteins were cloned into three different plasmids. Unique restriction enzymes sites are shown to highlight the modular design of the various expression plasmids. Resulting DNA constructs coded for the following fusion proteins: KRAB-NLS-6ZFP−3xmyc (SEQ ID NO: 8), SID-NLS-6ZFP−3xmyc, NLS-6ZFP-GGSGGS (SEQ ID NO: 9) linker-KRAB A-3xmyc, and NLS-6ZFP-GGSGGS linker-VP64-3xmyc.

FIG. 7: Regulation of Human Endothelin Receptor A (ETRA) Activity by Artificial Transcription Factors AO74A, AO74E, AO74R and AO74V

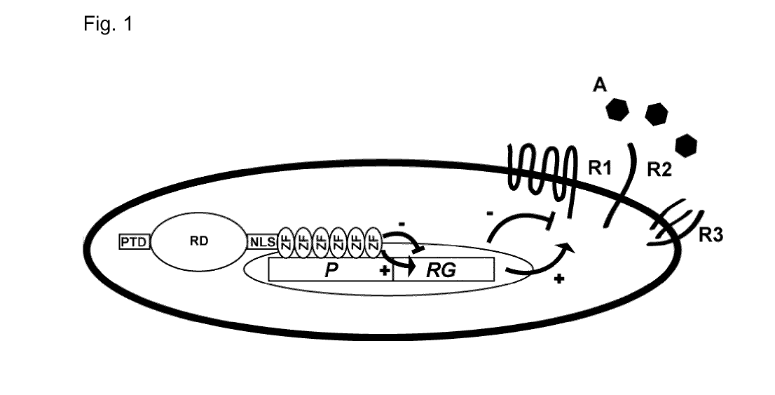

(A) Artificial transcription factor dependent repression of ETRA promoter-driven protein expression. Shown is the result of a luciferase reporter assay (RLuA=relative luciferase activity, in % relative to control C) following expression of AO74A (SEQ ID NO: 10), AO74E (SEQ ID NO: 11), AO74R (SEQ ID NO: 12), and AO74V (SEQ ID NO: 13) directed against target sites within the ETRA promoter. C=Yellow fluorescent protein (YFP) as control.

(B) AO74Vp (SEQ ID NO: 14), the transducible AO74V protein, does not inhibit HeLa cell proliferation compared to control B (buffer treated cells). RP=relative proliferation in % of control.

(C) AO74Vp does not inhibit proliferation of human uterine smooth muscle cells (hUtSMC) compared to control B (buffer treated cells).

(D) AO74Vp blocks ET-1-dependent contraction of hUtSMC. hUtSMC were embedded into 3-dimensional collagen lattices. C=cells treated with buffer as control. B=cells treated with buffer and ET-1. AO74Vp=cells treated with AO74Vp and ET-1. RLA=relative lattice area in % of control C. Details are described below.

FIG. 8: Enhancement of ETRA Promoter Activity Driven by Artificial Transcription Factors AO74Ra and AO74Va

(A) ETRA promoter-driven expression of luciferase reporter is increased following expression of the activating artificial transcription factors AO74Ra (SEQ ID NO: 15) and AO74Va (SEQ ID NO: 16). RLuA=relative luciferase activity, in % relative to control C, YFP.

(B) Treatment with AO74Vap (SEQ ID NO: 17) does not inhibit hUtSMCs cell proliferation. AO74Vp delivered as protein is not toxic to hUtSMCs cells and does not negatively impact cellular proliferation. B=buffer treated cells. RP=relative proliferation in % of control.

FIG. 9: Repression of Human Endothelin Receptor B (ETRB) Promoter Activity by AO1149N and AO1149P

Expression of artificial transcription factor AO1149N (SEQ ID NO: 18) and AO1149P (SEQ ID NO: 19) represses ETRB promoter activity compared to YFP (control C) in a luciferase reporter assay. RLuA=relative luciferase activity, in % relative to control C.

FIG. 10: Modulation of Human Toll-Like Receptor 4 (TLR4) Activity by AO55B and AO55E

(A) Expression of AO55B (SEQ ID NO: 20) and AO55E (SEQ ID NO: 21) blocks TLR4 promoter activity compared to YFP (control C) in a luciferase reporter assay. RLuA=relative luciferase activity, in % relative to control C.

(B) TLR4-dependent, LPS-induced secretion of interleukin (IL)-6 is blunted following expression of AO55B in macrophage-like U937 cells.

(C) Treatment with AO55 Bp (SEQ ID NO: 22) does not inhibit HeLa cell proliferation. RP=relative proliferation in % of control. B=buffer treated cells. RP=relative proliferation in % of control.

FIG. 11: High-Affinity IgE Receptor is Regulated by AO147A

(A) High-affinity IgE receptor alpha subunit (FCER1A) promoter-driven expression of a luciferase reporter is inhibited in rat basophilic RBL-2H3 cells following expression of AO147A (SEQ ID NO: 23). RLuA=relative luciferase activity, in % relative to control C, YFP.

(B) AO147Ap (SEQ ID NO: 24) does not inhibit HeLa cell proliferation. B=buffer treated cells. RP=relative proliferation in % of control.

C). Treatment with AO147Ap inhibits binding of human IgE to human basophilic KU812F cells by around 80%. IgEB=IgE bindability to FCER1 determined by flow cytometry using human IgE and mouse anti-human IgE labeled with FITC, in % compared to buffer-treated cells as control (B).

### DETAILED DESCRIPTION OF THE INVENTION

The invention relates to an artificial transcription factor comprising a polydactyl zinc finger protein targeting specifically a receptor gene promoter fused to an inhibitory or activatory protein domain, a nuclear localization sequence, and a protein transduction domain, and to pharmaceutical compositions comprising such an artificial transcription factor.

Treatment of many diseases is based on modulating cellular receptor signaling. Examples are high blood pressure where beta blockers inhibit the function of the beta adrenergic receptors, depression where serotonin uptake blockers increase agonist concentration and thus serotonin receptor signaling or glaucoma where prostaglandin analogues activate prostaglandin receptors in turn decreasing intraocular pressure. Traditionally, small molecules either in the form of receptor agonist or antagonists are used to impact receptor signaling for therapeutic purposes. However, cellular receptor signaling can also be influenced by direct modulation of receptor protein expression.

Pathological processes amenable to direct modulation of receptor expression levels are, for example, the following: Patients with congestive heart failure due to congenital heart disease will benefit from the upregulation of beta-adrenoceptors, since downregulation of this receptor in the myocardium is associated with the risk of post-operative heart failure. In Parkinson's disease, treatment with dopaminergic medication suppresses the availability of dopamine receptors, thus, upregulation of dopamine receptor will improve the efficacy of dopaminergic medication. In epilepsy, insufficient expression of cannabinoid receptors in the hippocampus is involved in disease etiology, thus, upregulation of cannabinoid receptor will be a viable therapy for epileptic patients.

For genetic diseases caused by haploinsufficiency of a receptor protein, such as insulin-like growth factor I receptor causing growth retardation, but also others, additional activation of the remaining functional receptor gene will be beneficial for the patient. Furthermore and among others, induction and perpetuation of pathological autoimmunity is connected to inappropriate signaling from Toll-like receptors. Thus, downregulation of Toll-like receptors breaks the vicious cycle of various autoimmune diseases. In allergic disease, prevention of the IgE-mediated signaling through the high-affinity IgE receptor is useful to manage allergic reactions. In cancer, downregulation of growth factor receptors or upregulation of extracellular matrix receptors are beneficial for the prevention of tumor progression.

Among such receptor molecules are proteins from the so called seven-transmembrane or G protein coupled receptor (GPCR) family of proteins, characterized by seven transmembrane domains anchoring the receptor in the plasma membrane and a G protein dependent signaling cascade. Examples for such proteins are receptors A and B for endothelin. Other receptor proteins are anchored via a single transmembrane region, for example the receptor for lipopolysaccharide, Toll-like receptor 4, or various cytokine receptors such as IL-4 receptor. Other receptors consist of multimeric protein complexes, for example the high-affinity receptor for IgE antibodies that consists of alpha, beta and gamma chains, or the T-cell receptor consisting of alpha, beta, gamma, delta, epsilon and zeta chains. Thus, subsumed under the term “receptor molecule” are proteins from different protein families with very different modes of action.

Receptors considered in the present invention are human receptor molecules encoded by HTR1A, HTR1B, HTR1D, HTR1E, HTR1F, HTR2A, HTR2B, HTR2c, HTR4, HTR5A, HTR5BP, HTR6, HTR7, CHRM1, CHRM2, CHRM3, CHRM4, CHRM5, ADORA1, ADORA2A, ADORA2B, ADORA3, ADRA 1A, ADRA1B, ADRA1D, ADRA2A, ADRA2B, ADRA2C, ADRB1, ADRB2, ADRB3, AGTR1, AGTR2, APLNR, GPBAR1, NMBR, GRPR, BRS3, BDKRB1, BDKRB2, CNR1, CNR2, CCR1, CCR2, CCR3, CCR4, CCR5, CCR6, CCR7, CCR8, CCR9, CCR10, CXCR1, CXCR2, CXCR3, CXCR4, CXCR5, CXCR6, CXCR7, CX3CR1, XCR1, CCKAR, CCKBR, C3AR1, C5AR1, GPR77, DRD1, DRD2, DRD3, DRD4, DRD5, EDNRA, EDNRB, GPER, FPR1, FPR2, FPR3, FFAR1, FFAR2, FFAR3, GPR42, GALR1, GALR2, GALR3, GHSR, FSHR, LHCGR, TSHR, GNRHR, GNRHR2, HRH1, HRH2, HRH3, HRH4, HCAR1, HCAR2, HCAR3, KISS1R, LTB4R, LTB4R2, CYSLTR1, CYSLTR2, OXER1, FPR2, LPAR1, LPAR2, LPAR3, LPAR4, LPAR5, S1PR1, S1PR2, S1PR3, S1PR4, S1PR5, MCHR1, MCHR2, MC1R, MC2R, MC3R, MC4R, MC5R, MTNR1A, MTNR1B, MLNR, NMUR1, NMUR2, NPFFR1, NPFFR2, NPSR1, NPBWR1, NPBWR2, NPY1R, NPY2R, PPYR1, NPY5R, NPY6R, NTSR1, NTSR2, OPRD1, OPRK1, OPRM1, OPRL1, HCRTR1, HCRTR2, P2RY1, P2RY2, P2RY4, P2RY6, P2RY11, P2RY12, P2RY13, P2RY14, QRFPR, PTAFR, PROKR1, PROKR2, PRLHR, PTGDR, PTGDR2, PTGER1, PTGER2, PTGER3, PTGER4, PTGFR, PTGIR, TBXA2R, F2R, F2RL1, F2RL2, F2RL3, RXFP1, RXFP2, RXFP3, RXFP4, SSTR1, SSTR2, SSTR3, SSTR4, SSTR5, TACR1, TACR2, TACR3, TRHR, TAAR1, UTS2R, AVPR1A, AVPR1B, AVPR2, OXTR, CCRL2, CMKLR1, GPR1, GPR3, GPR4, GPR6, GPR12, GPR15, GPR17, GPR18, GPR19, GPR20, GPR21, GPR22, GPR25, GPR26, GPR27, GPR31, GPR32, GPR33, GPR34, GPR35, GPR37, GPR37L1, GPR39, GPR42, GPR45, GPR50, GPR52, GPR55, GPR61, GPR62, GPR63, GPR65, GPR68, GPR75, GPR78, GPR79, GPR82, GPR83, GPR84, GPR85, GPR87, GPR88, GPR101, GPR119, O3FAR1, GPR132, GPR135, GPR139, GPR141, GPR142, GPR146, GPR148, GPR149, GPR150, GPR151, GPR152, GPR153, GPR160, GPR161, GPR162, GPR171, GPR173, GPR174, GPR176, GPR182, GPR183, LGR4, LGR5, LGR6, LPAR6, MAS1, MAS1L, MRGPRD, MRGPRE, MRGPRF, MRGPRG, MRGPRX1, MRGPRX2, MRGPRX3, MRGPRX4, OPN3, OPN5, OXGR1, P2RY8, P2RY10, SUCNR1, TAAR2, TAAR3, TAAR4P, TAAR5, TAAR6, TAAR8, TAAR9, CCBP2, CCRL1, DARC, CALCR, CALCRL, CRHR1, CRHR2, GHRHR, GIPR, GLP1R, GLP2R, GCGR, SCTR, PTH1R, PTH2R, ADCYAP1R1, VIPR1, VIPR2, BAI1, BAI2, BAI3, CD97, CELSR1, CELSR2, CELSR3, ELTD1, EMR1, EMR2, EMR3, EMR4P, GPR56, GPR64, GPR97, GPR98, GPR110, GPR111, GPR112, GPR113, GPR114, GPR115, GPR116, GPR123, GPR124, GPR125, GPR126, GPR128, GPR133, GPR144, GPR157, LPHN1, LPHN2, LPHN3, CASR, GPRC6A, GABBR1, GABBR2, GRM1, GRM2, GRM3, GRM4, GRM5, GRM6, GRM7, GRM8, GPR156, GPR158, GPR179, GPRC5A, GPRC5B, GPRC5C, GPRC5D, TAS1R1, TAS1R2, TAS1R3, FZD1, FZD2, FZD3, FZD4, FZD5, FZD6, FZD7, FZD8, FZD9, FZD10, SMO, GPR107, GPR137, OR51E1, TPRA1, GPR143, THRA, THRB, RARA, RARB, RARG, PPARA, PPARD, PPARG, NR1D1, NR1D2, RORA, RORB, RORC, NR1H4, NR1H5P, NR1H3, NR1H2, VDR, NR112, NR113, HNF4A, HNF4G, RXRA, RXRB, RXRG, NR2C1, NR2C2, NR2E1, NR2E3, NR2F1, NR2F2, NR2F6, ESR1, ESR2, ESRRA, ESRRB, ESRRG, AR, NR3C1, NR3C2, PGR, NR4A1, NR4A2, NR4A3, NR5A1, NR5A2, NR6A1, NROB1, NROB2, HTR3A, HTR3B, HTR3c, HTR3D, HTR3E, GABRA1, GABRA2, GABRA3, GABRA4, GABRA5, GABRA6, GABRB1, GABRB2, GABRB3, GABRG1, GABRG2, GABRG3, GABRD, GABRE, GABRQ, GABRP, GABRR1, GABRR2, GABRR3, GLRA1, GLRA2, GLRA3, GLRA4, GLRB, GRIA1, GRIA2, GRIA3, GRIA4, GRID1, GRID2, GRIK1, GRIK2, GRIK3, GRIK4, GRIK5, GRIN¹, GRIN2A, GRIN2B, GRIN2C, GRIN2D, GRIN3A, GRIN3B, CHRNA1, CHRNA2, CHRNA3, CHRNA4, CHRNA5, CHRNA6, CHRNA7, CHRNA9, CHRNA10, CHRNB1, CHRNB2, CHRNB3, CHRNB4, CHRNG, CHRND, CHRNE, P2RX1, P2RX2, P2RX3, P2RX4, P2RX5, P2RX6, P2RX7, ZACN, AGER, TLR1, TLR2, TLR3, TLR4, TLR5, TLR6, TLR7, TLR8, TLR9, TLR10, TLR11, LILRA1, LILRA2, LILRA3, LILRA4, LILRA5, LILRA6, LILRB1, LILRB2, LILRB3a, LILRB4, LILRB5, LILRB6, LILRB7, EGFR, ERBB2, ERBB3, ERBB4, GFRa1, GFRa2, GFRa3, GFRa4, NPR1, NPR2, NPR3, NPR4, NGFR, NTRK1, NTRK2, NTRK3, EGFR, ERB2, ERB3, ERB4, INSR, IRR, IG1R, PDGFalpha, PDGFbeta, Fms, Kit, Flt3, FGFR1, FGFR2, FGFR3, FGFR4, BFR2, VGR1, VGR2, VGR3, EPA1, EPA2, EPA3, EPA4, EPA5, EPA7, EPA8, EPB1, EPB2, EPB3, EPB4, EPB6, TrkA, TrkB, TrkC, UFO, TYRO3, MERK, TIE1, TIE2, RON, MET, DDR1, DDR2, RET, ROS, LTK, ROR1, ROR2, RYK, PTK7, and KIT.

Further receptors considered are human receptors recognizing interleukin (IL)-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, leptin, interferon-alpha, interferon-beta, interferon-gamma, tumor necrosis factor alpha, lymphotoxin, prolactin, oncostatin M, leukemia inhibitory factor, colony-stimulating factor, immunoglobulin A, immunoglobulin D, immunoglobulin G, immunoglobulin M, immunoglobulin E, human leukocyte antigen (HLA) A, HLA-B, HLA-C, HLA-E, HLA-F, HLA-G, HLA-DP, HLA-DQ, HLA-DR, transforming growth factor alpha, transforming growth factor beta, nerve growth factor, brain-derived neurotrophic factor, neurotrophin-3, neurotrophin-4, adrenomedullin, angiopoietin, autocrine motility factor, bone morphogenetic proteins, erythropoietin, fibroblast growth factor, glial cell line-derived neurotrophic factor, granulocyte colony-stimulating factor, granulocyte macrophage colony-stimulating factor, growth differentiation factor-9, hepatocyte growth factor, hepatoma-derived growth factor, insulin-like growth factor, insulin, migration-stimulating factor, myostatin, platelet-derived growth factor, thrombopoietin, vascular endothelial growth factor, placental growth factor, and growth hormone.

Further considered are receptors encoded by homologous non-human genes, for example by porcine, equine, bovine, feline, canine, or murine genes; and receptors encoded by homologous plant receptor genes, for example genes found in crop plants such as wheat, barley, corn, rice, rye, oat, soybean, peanut, sunflower, safflower, flax, beans, tobacco, or life-stock feed grasses, and genes found in fruit plants such as apple, pear, banana, citrus fruit, grape or the like.

Retroviruses have exceptionally high potential for immunogenicity, thus limiting their use in repeated application of a certain treatment. Due to the high conservation of zinc finger modules such an immune reaction will be minor or absent following application of artificial transcription factors of the invention, or might be avoided or further minimized by small changes to the overall structure eliminating immunogenicity while still retaining target site binding and thus function. Furthermore, modification of artificial transcription factors of the invention with polyethylene glycol is considered to reduce immunogenicity. In addition, application of artificial transcription factors of the invention to immune privileged organs such as the eye and the brain will avoid any immune reaction, and induce whole body tolerance to the artificial transcription factors. For the treatment of chronic diseases outside of immune privileged organs, induction of immune tolerance through prior intraocular injection is considered.

Identification of small molecules for the modulation of receptor activity mostly relies on extensive and time-consuming screening procedures among a wide variety of different molecules from different classes of substances. Especially, fast, rational design of such small molecule drugs for a given receptor molecule is quite challenging. In contrast, artificial transcription factors of the invention all belong to the same substance class with a highly defined overall composition. Two hexameric zinc finger protein-based artificial transcription factors targeting two very diverse promoter sequences still have a minimal amino acid sequence identity of 85% with an overall similar tertiary structure and can be generated via a standardized method (as described below) in a fast and economical manner. Thus, artificial transcription factors of the invention combine, in one class of molecule, exceptionally high specificity for a very wide and diverse set of targets with overall similar composition. In addition, formulation of artificial transcription factors of the invention into drugs can rely on previous experience further expediting the drug development process.

Protein transduction domain (PTD) mediated, intracellular delivery of artificial transcription factors is a new way of taking advantage of the high selectivity of biologicals to target receptor molecules in a novel fashion. While conventional drugs modulate the activity of certain receptors, artificial transcription factors alter the availability of these proteins. And since artificial transcription factors are tailored to act specifically on the promoter region of such receptor genes, the invention allows selectively targeting even closely related proteins. This is based on the only loose conservation of the promoter regions even of closely related proteins. The protein transduction domain-mediated delivery of artificial transcription factors is useful to modulate the reaction of cells to external stimuli including but not limited to hormones as for example insulin, endothelin or immunomodulatory peptides such as interleukins, chemokines and cytokines, but also antibodies, antigens and molecular patterns. But also the cellular response towards other soluble signaling molecules such as glutamate or gamma-amino butyric acid and other neurotransmitters can be modulated by this approach. Taking advantage of the high selectivity of the artificial transcription factors according to the invention, even a tissue-specific targeting of a drug action is possible based on the oftentimes tissue-specific expression of certain members of a given receptor protein family.

The invention also relates to the use of such artificial transcription factors in treating diseases modulated by the binding of specific effectors to receptors, for which the polydactyl zinc finger protein is specifically targeting the receptor gene promoter. Likewise the invention relates to a method of treating diseases comprising administering a therapeutically effective amount of an artificial transcription factor to a patient in need thereof, wherein the disease to be treated is modulated by the binding of specific effectors to receptors, for which the polydactyl zinc finger protein is specifically targeting the receptor gene promoter.

Polydactyl zinc finger proteins considered are tetrameric, pentameric, hexameric or heptameric zinc finger proteins. “Tetrameric”, “pentameric”, “hexameric” and “heptameric” means that the zinc finger protein consists of four, five, six, and seven partial protein structures, respectively, each of which has binding specificity for a particular nucleotide triplet. Preferably the artificial transcription factors comprises a hexameric zinc finger protein.

Selection of Target Sites within a Given Promoter Region

Target site selection is crucial for the successful generation of a functional artificial transcription factor. For an artificial transcription factor to modulate target gene expression in vivo, it must bind its target site in the genomic context of the target gene. This necessitates the accessibility of the DNA target site, meaning chromosomal DNA in this region is not tightly packed around histones into nucleosomes and no DNA modifications such as methylation interfere with artificial transcription factor binding. While large parts of the human genome are tightly packed and transcriptionally inactive, the immediate vicinity of the transcriptional start site (−1000 to +200 bp) of an actively transcribed gene must be accessible for endogenous transcription factors and the transcription machinery such as

RNA polymerases. Thus, selecting a target site in this area of any given target gene will greatly enhance the success rate for the generation of an artificial transcription factor with the desired function in vivo.

Selection of Target Sites within the Human Endothelin Receptor A (ETRA) Promoter Region

Hexameric zinc finger proteins (6ZFPs) targeting specifically the endothelin receptor A promoter are determined by analysing the human ETRA gene as follows:

The human ETRA gene (genomic region containing the promoter region SEQ ID NO: 25, coding region SEQ ID NO: 26) is comprised of eight exons separated by seven introns (Hosoda K. et al., 1992, J Biol Chem 267, 18797-18804). Exon 1 and intron 1 are located in the 5′ non-coding region, the transcription start site is 502 bp upstream of the ATG translation initiation codon.

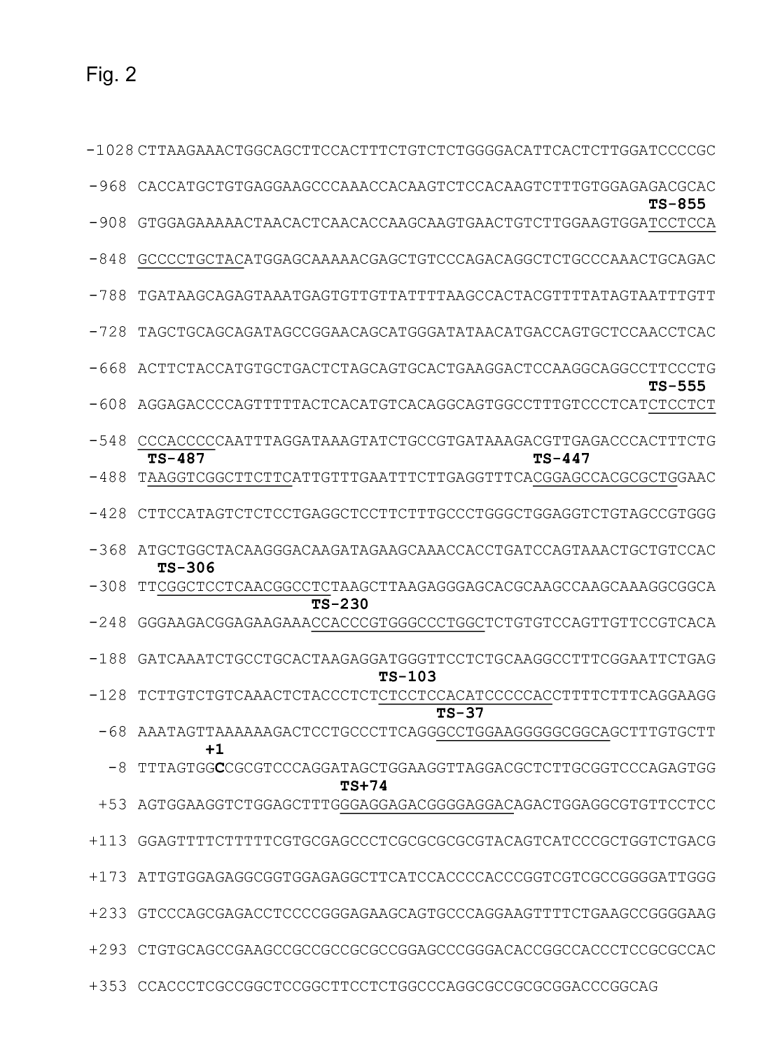

The ETRA promoter region from −1000 bp to +100 bp relative to the transcription start site was analyzed for (GNN)₆ target sites (FIG. 2 and Table 1). Using ZiFiT software (Sander J. D. et al., 2010, Nucleic Acids Res 38, W462-468), TS−855 and TS+74 were identified and GNN zinc finger modules of the Barbas set were chosen to design ZFP−855A and ZFP+74A.

Although the rational design of zinc finger proteins based on preselected zinc finger modules is known, screening approaches based on unbiased ZFP-containing libraries proved superior for the identification of high-affinity zinc finger proteins. Therefore, an additional (GNN)₆ sequence (TS−103), initially excluded by the ZiFiT program, was selected. Furthermore, other 18 bp target sites containing GNN or CNN triplets were selected between TS−855 and TS+74. In addition, 15 bp target sites were selected between TS−855 and TS−306 for screening with 6ZFP libraries.

Selection of Target Sites within the Human Endothelin Receptor B (ETRB) Promoter Region

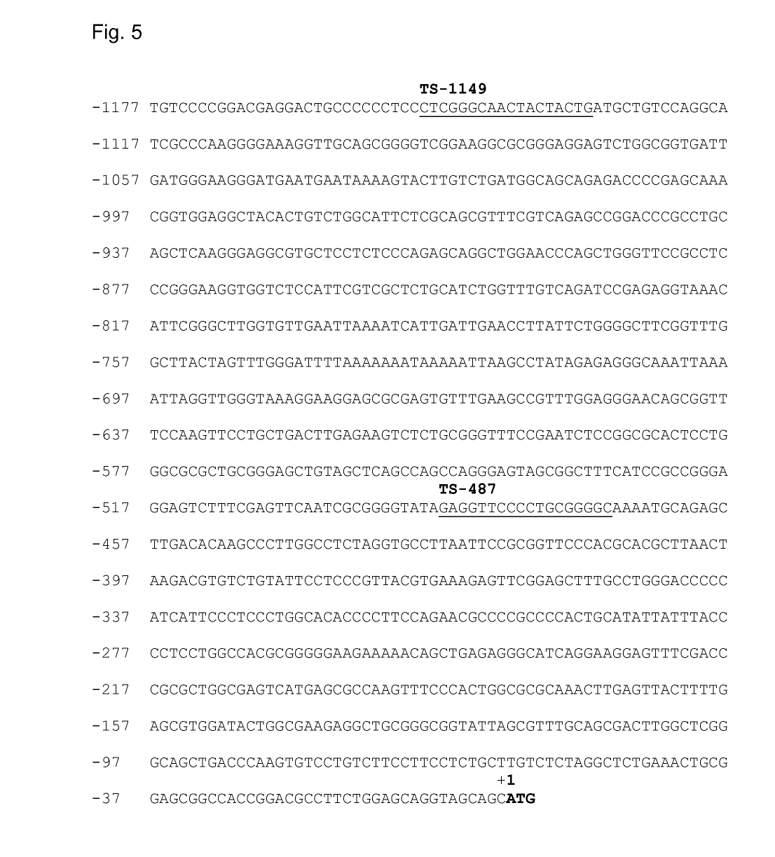

Binding sites for artificial transcription factors for regulating ETRB expression were selected as follows: The 5′ region of the ETRB gene (SEQ ID NO: 27) contains putative transcriptional start sites at −1195, −817, −229 and −258 bp upstream of the translational start site. Therefore, 18 bp target sites consisting of GNN or CNN triplets were selected between −1149 bp and −487 bp (see FIG. 5).

Selection of Target Sites within the Human Toll-Like Receptor 4 (TLR4) Promoter Region

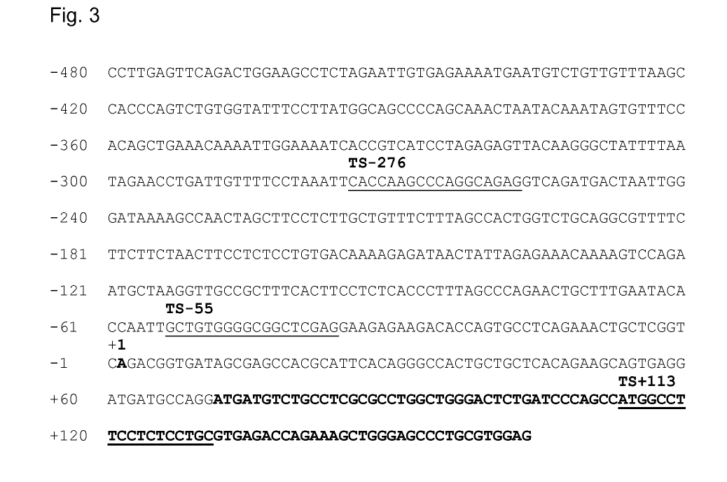

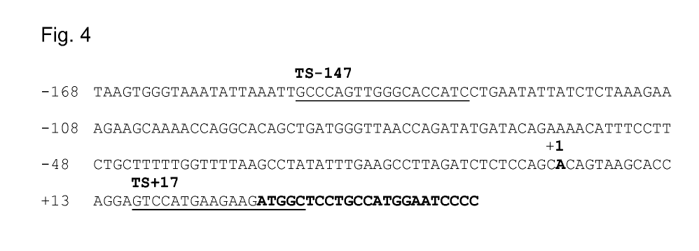

18 bp potential binding sites for TLR4-expression regulating artificial transcription factors consisting of six G/CNN triplets were selected in the 5′ region of the TLR4 gene (SEQ ID NO: 28) between −276 bp and +113 bp relative to the transcription start site (see FIG. 3).

Selection of Target Sites within the Human High-Affinity IgE Receptor A (FCER1A) Promoter Region

Binding sites for FCER1A expression-regulating artificial transcription factors were selected in the 5′ region of the FCER1A gene (SEQ ID NO: 29). The human FCER1A promoter contains a proximal regulatory region around 200 bp upstream of the transcriptional start site as well as a distal region further upstream containing IL-4 responsive elements (Nishiyama C., 2006, Biosci Biotechnol Biochem 70 (1), 1-9). Potential binding sites for FCER1A-regulating artificial transcription factors were selected in the proximal regulatory region at −147 bp and +17 bp relative to the transcriptional start site.

### a Modified Yeast One Hybrid (Y1H) Screen to Select Hexamerixc Zinc Finger Proteins

Based on the library cloning scheme published by Gonzalez B. et al., 2010, Nat Protoc 5, 791-810, the yeast shuttle vector pGAD10 (pAN1025) was modified to allow for efficient generation of zinc finger protein coding libraries. To improve cloning efficiency, initial assembly of zinc finger protein coding libraries was done in pBluescript followed by transfer of the libraries into pAN1025. Using sequential digest and DNA dephosphorylation, the formation of head-head or tail-tail ligated zinc finger modules was prevented, thus improving effective library coverage.

Conventional Y1H screening is aimed at identifying transcription factors for a given DNA sequence out of a relatively small pool of naturally occurring proteins. The goal here was to select hexameric zinc finger proteins (6ZFPs) from a very large pool of proteins (around 16*10⁶) all with the potential of binding to the used target site. This necessitates the use of additional selection pressure to identify 6ZFPs with the highest target site affinity. While Aureobasidin A (AbA) concentrations of 200 ng/ml are typically used for conventional Y1H analyses, up to 4000 ng/ml AbA were useful to improve selection above what is normally achieved with the employed Y1H system (MatchMaker Gold, Clontech).

To increase selection pressure further and thus to identify 6ZFPs with even higher binding capacity to a given target site, the Y1H system was further modified. For the first round of selection, artificial transcription factor libraries were contained in yeast vectors based on the 2μ origin of replication. Such vectors replicate independently inside yeast cells to about 50 copies, leading to a strong production of 6ZFPs. For a second round of selection, the artificial transcription factor libraries were contained on yeast vectors based on a low-copy ARS/CEN vector with a copy number of 1-2/cell. Due to the lower expression level of library zinc finger proteins, ARS/CEN-based Y1H screens combined with 4000 ng/ml of AbA are more sensitive and yield 6ZFPs with higher binding affinity for their cognate target sequence.

TABLE 1
Target sites within ETRA promoter region and results from Y1H screens

| | ZFPs | | SEQ |
|---|---|---|---|
| Target site DNA seq. | from Y1H | ZF modules of isolated ZFPs | ID |
| 5′-3′ ^{a}) | screen ^{b}) | (F1 -F2-F3-F4-F5-F6) ^{c}) | NO^{d}) |
| −855 | ZFP − 855A | GM03-GM03-GM14-GM11-GM02-GM04 | 31 |
| TCCTCCAGCCCCTGCTAC | ZFP − 855B | GM15-GM03-GM04-GM15-GM02-GM04 | 32 |
| (SEQ ID NO: 30) | ZFP − 855C | GM15-GM11-GM08-GM11-GM02-GM04 | 33 |
| | ZFP − 855D | GM02-GM02-GM02-GM15-GM11-GM16 | 34 |
| | ZFP − 855E | GM03-GM15-GM03-GM08-GM11-GM04 | 35 |
| | ZFP − 855F | GM03-GM07-GM16-GM15-GM02-GM04 | 36 |
| | ZFP − 855G | GM15-GM03-GM06-GM10-GM04-GM04 | 37 |
| −555 | ZFP − 555A | GM15-GM11-GM03-GM12-GM11-GM15 | 39 |
| CTCCTCTCCCACCCC | ZFP − 555B | GM09-GM03-GM11-GM11-GM10-GM06 | 40 |
| (SEQ ID NO: 38) | ZFP − 555C | GM15-GM02-GM12-GM02-GM09-GM06 | 41 |
| | ZFP − 555D | GM09-GM07-GM12-GM03-GM12-GM06 | 42 |
| | ZFP − 555E | GM08-GM03-GM09-GM03-GM11-GM09 | 43 |
| −487 | ZFP − 487A | CM04-GM13-GM12-GM02-GM16-GM09 | 45 |
| AAGGTCGGCTTCTTC | ZFP − 487B | CM15-GM16-GM06-GM01-GM01-GM13 | 46 |
| (SEQ ID NO: 44) | ZFP − 487C | CM12-GM13-GM06-GM01-GM01-GM16 | 47 |
| | ZFP − 487D | CM13-GM10-GM01-GM01-GM14-GM05 | 48 |
| | ZFP − 487E | CM15-GM06-GM06-GM01-GM01-GM16 | 49 |
| | ZFP − 487F | CM11-GM09-GM16-GM05-GM04-GM04 | 50 |
| −447 | ZFP − 447A | GM16-CM14-GM10-GM13-CM09-CM11 | 52 |
| CGGAGCCACGCGCTG | | | |
| (SEQ ID NO: 51) | | | |
| -306 | ZFP − 306A | CM15-GM04-GM09-GM16-GM05-GM04 | 54 |
| CGGCTCCTCAACGGCCTC | ZFP − 306B | CM11-GM04-GM03-GM09-GM04-GM05 | 55 |
| (SEQ ID NO: 53) | ZFP − 306C | CM13-GM04-GM03-GM09-GM01-GM06 | 56 |
| | ZFP − 306D | CM05-GM04-GM03-GM09-GM01-GM06 | 57 |
| −230 | ZFP − 230A | GM03-CM09-GM09-GM05-CM11-CM15 | 59 |
| CCACCCGTGGGCCCTGGC | ZFP − 230B | GM12-CM11-GM04-GM07-CM03-CM08 | 60 |
| (SEQ ID NO: 58) | ZFP − 230C | GM03-CM07-GM03-GM02-CM15-CM11 | 61 |
| | ZFP − 230D | GM12-CM11-GM04-GM07-CM07-CM12 | 62 |
| | ZFP − 230E | GM13-CM09-GM13-GM13-CM11-CM12 | 63 |
| | ZFP − 230F | GM13-CM09-GM07-GM04-CM11-CM12 | 64 |
| −103 | ZFP − 103A | GM15-GM08-GM14-GM13-GM11-GM16 | 66 |
| CTCCTCCACATCCCCCAC | ZFP − 103B | GM07-GM12-GM13-GM11-GM02-GM07 | 67 |
| (SEQ ID NO: 65) | ZFP − 103C | GM06-GM15-GM12-GM13-GM11-GM07 | 68 |
| | ZFP − 103D | GM10-GM12-GM13-GM11-GM16-GM08 | 69 |
| | ZFP − 103E | GM15-GM07-GM12-GM13-GM11-GM06 | 70 |
| | ZFP − 103F | GM03-GM06-GM12-GM13-GM11-GM05 | 71 |
| | ZFP − 103G | GM14-GM10-GM13-GM11-GM11-GM07 | 72 |
| | ZFP − 103H | GM01-GM12-GM10-GM14-GM11-GM10 | 73 |
| | ZFP − 103I | GM07-GM06-GM12-GM13-GM11-GM16 | 74 |
| | ZFP − 103J | GM05-GM10-GM13-GM11-GM08-GM06 | 75 |
| | ZFP − 103K | GM07-GM10-GM13-GM11-GM11-GM08 | 76 |
| | ZFP − 103L | GM10-GM12-GM13-GM11-GM16-GM06 | 77 |
| | ZFP − 103M | GM10-GM12-GM13-GM11-GM04-GM06 | 78 |
| | ZFP − 103N | GM03-GM07-GM10-GM13-GM11-GM08 | 79 |
| | ZFP − 103O | GM05-GM04-GM13-GM11-GM08 | 80 |
| −37 | ZFP − 37A | GM14-GM04-GM04-GM12-CM11-GM07 | 82 |
| GGCCTGGAAGGGGGCGGC | ZFP − 37B | GM02-GM02-GM12-GM13-CM11-GM12 | 83 |
| (SEQ ID NO: 81) | ZFP − 37C | GM08-GM13-GM04-GM06-CM11-GM09 | 84 |
| | ZFP − 37D | GM04-GM04-GM06-GM11-CM03-GM04 | 85 |
| | ZFP − 37E | GM02-GM02-GM13-GM11-CM14-GM07 | 86 |
| | ZFP − 37F | GM11-GM01-GM12-GM07-CM15-GM06 | 87 |
| | ZFP − 37G | GM02-GM02-GM02-GM11-CM06-GM07 | 88 |
| | ZFP − 37H | GM12-GM16-GM04-GM06-CM11-GM09 | 89 |
| | ZFP − 37I | GM03-GM07-GM11-GM13-CM14-GM06 | 90 |
| | ZFP − 37J | GM06-GM13-GM04-GM06-CM11-GM09 | 91 |
| | ZFP − 37K | GM15-GM07-GM12-GM13-CM11-GM15 | 92 |
| | ZFP − 37L | GM15-GM14-GM02-GM10-CM11-GM08 | 93 |
| | ZFP − 37M | GM08-GM02-GM10-GM11-CM14-GM06 | 94 |
| | ZFP − 37N | GM02-GM02-GM12-GM13-CM11-GM03 | 95 |
| +74 | ZFP + 74A | GM05-GM09-GM11-GM05-GM03-GM03 | 97 |
| GGAGGAGACGGGGAGGAC | ZFP + 74B | GM01-GM12-GM07-GM05-GM03-GM07 | 98 |
| (SEQ ID NO: 96) | ZFP + 74C | GM09-GM11-GM08-GM05-GM11-GM07 | 99 |
| | ZFP + 74D | GM09-GM09-GM04-GM02-GM12-GM09 | 100 |
| | ZFP + 74E | GM12-GM11-GM11-GM05-GM03-GM13 | 101 |
| | ZFP + 74F | GM16-GM09-GM07-GM05-GM04-GM07 | 102 |
| | ZFP + 74G | GM09-GM15-GM05-GM02-GM07-GM06 | 103 |
| | ZFP + 74H | GM09-GM07-GM05-GM15-GM03-GM06 | 104 |
| | ZFP + 74I | GM09-GM07-GM08-GM03-GM03-GM10 | 105 |
| | ZFP + 74J | GM09-GM12-GM06-GM03-GM03-GM07 | 106 |
| | ZFP + 74K | GM08-GM09-GM06-GM05-GM03-GM07 | 107 |
| | ZFP + 74L | GM16-GM05-GM06-GM09-GM11-GM06 | 108 |
| | ZFP + 74M | GM08-GM09-GM12-GM14-GM05-GM12 | 109 |
| | ZFP + 74N | GM15-GM09-GM08-GM07-GM03-GM03 | 110 |
| | ZFP + 74O | GM09-GM11-GM07-GM05-GM13-GM16 | 111 |
| | ZFP + 74P | GM11-GM05-GM12-GM12-GM04-GM03 | 112 |
| | ZFP + 74Q | GM05-GM12-GM09-GM05-GM16-GM07 | 113 |
| | ZFP + 74R | GM09-GM10-GM05-GM03-GM07-GM07 | 114 |
| | ZFP + 74S | GM05-GM12-GM15-GM05-GM12-GM16 | 115 |
| | ZFP + 74T | GM01-GM13-GM12-GM15-GM07-GM12 | 116 |
| | ZFP + 74U | GM09-GM11-GM06-GM03-GM07-GM08 | 117 |
| | ZFP + 74V | GM09-GM12-GM15-GM05-GM04-GM14 | 118 |

^{a}) Shown are ETRA promoter target sites (named according to their distance to the transcription start site) in column 1.
^{b}) Column 2 names the ZFPs identified in a Y1H screen to bind to ETRA promoter target sites. The naming scheme is as follows: ZFP followed by the name of the target site and a letter designating different ZFP isolated in the screen.
^{c}) Column 3 shows the constitution of the ZFPs by detailing the individual zinc finger modules according to their established binding preference. GM01 designates a zinc finger module preferably binding to a GAA, GM02 to GCA, GM03 to GGA, GM04 to GTA, GM05 to GAC, GM06 to GCC, GM07 to GGC, GM08 to GTC, GM09 to GAG, GM10 to GCG, GM11 to GGG, GM12 to GTG, GM13 to GAT, GM14 to GCT, GM15 to GGT, GM16 to GTT, and furthermore, CM01 to CAC, CM02 to CAA, CM03 to CAG, CM04 to CAT, CM05 to CCA, CM06 to CCC, CM07 to CCG CM08 to CCT, CM09 to CGA, CM10 to CGC, CM11 to CGG, CM12 to CGT, CM13 to CTA, CM14 to CTG, and CM15 to CTT triplets.
^{d}) Column 4 refers to the sequence IDs of ZFPs identified to bind to the respective target site sequence.

TABLE 2
Target sites within ETRB promoter and results from Y1H screen

| | ZFPs from | | SEQ |
|---|---|---|---|
| Target site DNA seq. | Y1H screen | ZF modules of isolated ZFPs | ID |
| 5′-3′ ^{a}) | ^{b}) | (F1-F2-F3-F4-F5-F6) ^{c}) | NO^{d}) |
| −1149 | ZEB − 1149A | CM14-CM12-CM13-CM06-GM09-CM11 | 120 |
| CTCGGGCAACTACTACTG | ZEB − 1149B | CM14-CM08-CM11-CM11-GM09-CM11 | 121 |
| (SEQ ID NO: 119) | ZEB − 1149C | CM12-CM14-CM11-CM11-GM09-CM11 | 122 |
| | ZEB − 1149D | CM06-CM04-CM11-CM11-GM09-CM11 | 123 |
| | ZEB − 1149E | CM11-CM12-CM08-CM08-GM06-CM06 | 124 |
| | ZEB − 1149F | CM11-CM08-CM12-CM11-GM09-CM11 | 125 |
| | ZEB − 1149G | CM15-CM11-CM08-CM06-GM09-CM11 | 126 |
| | ZEB − 1149H | CM14-CM15-CM04-CM15-GM13-CM11 | 127 |
| | ZEB − 1149I | CM08-CM12-CM11-CM11-GM09-CM11 | 128 |
| | ZEB − 1149J | CM02-CM10-CM11-CM11-GM09-CM11 | 129 |
| | ZEB − 1149K | CM14-CM04-CM11-CM11-GM09-CM11 | 130 |
| | ZEB − 1149L | CM15-CM12-CM11-CM11-GM09-CM11 | 131 |
| | ZEB − 1149M | CM08-CM12-CM11-CM11-GM09-CM11 | 132 |
| | ZEB − 1149N | CM15-CM08-CM11-CM11-GM09-CM11 | 133 |
| | ZEB − 1149O | CM06-CM08-CM04-CM11-GM07-CM08 | 134 |
| | ZEB − 1149P | CM09-CM11-CM11-GM09-CM11 | 135 |
| | ZEB − 1149Q | CM15-CM14-CM12-CM12-GM04-CM08 | 136 |
| −487 | ZEB − 487A | GM03-CM11-CM14-CM12-GM16-GM13 | 138 |
| GAGGTTCCCCTGCGGGGC | ZEB − 487B | GM07-CM11-CM14-CM06-GM16-GM09 | 139 |
| (SEQ ID NO: 137) | ZEB − 487C | GM15-CM11-CM08-CM12-GM16-GM13 | 140 |
| | ZEB − 487D | GM03-CM11-CM15-CM12-GM16-GM09 | 141 |
| | ZEB − 487E | GM15-CM11-CM14-CM12-GM04-GM09 | 142 |
| | ZEB − 487F | GM03-CM11-CM14-CM12-GM04-GM13 | 143 |

^{a}) Shown are ETRB promoter target sites (named according to their distance to the translation start site) in column 1.
^{b}) Column 2 names the ZFPs identified in a Y1H screen to bind to ETRB promoter target sites. The naming scheme is as follows: ZEP followed by the name of the target site and a letter designating different ZFP isolated in the screen.
^{c}) Column 3 shows the constitution of the ZFPs by detailing the individual zinc finger modules according to their established binding preference. GM01 designates a zinc finger module preferably binding to a GAA, GM02 to GCA, GM03 to GGA, GM04 to GTA, GM05 to GAC, GM06 to GCC, GM07 to GGC, GM08 to GTC, GM09 to GAG, GM10 to GCG, GM11 to GGG, GM12 to GTG, GM13 to GAT, GM14 to GCT, GM15 to GGT, GM16 to GTT, and furthermore, CM01 to CAC, CM02 to CAA, CM03 to CAG, CM04 to CAT, CM05 to CCA, CM06 to CCC, CM07 to CCG CM08 to CCT, CM09 to CGA, CM10 to CGC, CM11 to CGG, CM12 to CGT, CM13 to CTA, CM14 to CTG, and CM15 to CTT triplets.
^{d}) Column 4 refers to the sequence IDs of ZFPs identified to bind to the respective target site sequence.

TABLE 3
Target sites within TLR4 promoter and results from Y1H screen

| | ZFPs from | | SEQ |
|---|---|---|---|
| Target site DNA seq. | Y1H screen | ZF modules of isolated ZFPs | ID |
| 5′-3′ ^{a}) | ^{b}) | (F1-F2-F3-F4-F5-F6) ^{c}) | NO^{d}) |
| −276 | ZFP − 276A | GM09-GM04-CM03-GM13-CM02-CM08 | 145 |
| CACCAAGCCCAGGCAGAG | ZFP − 276B | GM09-GM02-CM03-GM04-CM08-CM03 | 146 |
| (SEQ ID NO: 144) | ZFP − 276C | GM09-GM04-CM03-GM04-CM02-CM15 | 147 |
| | ZFP − 276D | GM09-GM02-CM14-GM04-CM02-CM14 | 148 |
| | ZFP − 276E | GM09-GM02-CM03-GM13-CM08-CM12 | 149 |
| | ZFP − 276F | GM09-GM02-CM03-GM16-CM02-CM02 | 150 |
| | ZFP − 276G | GM09-GM02-CM03-GM16-CM08-CM12 | 151 |
| | ZFP − 276H | GM11-GM02-CM03-GM16-CM02-CM12 | 152 |
| | ZFP − 276I | GM09-GM04-CM14-GM14-CM02-CM12 | 153 |
| −55 | ZFP − 55A | GM07-CM15-CM12-GM09-GM01-GM13 | 155 |
| GCTGTGGGGCGGCTCGAG | ZFP − 55B | GM03-CM09-CM04-GM16-GM09-GM07 | 156 |
| (SEQ ID NO: 154) | ZFP − 55C | GM07-CM15-CM06-GM14-GM06-GM12 | 157 |
| | ZFP − 55D | GM13-CM11-CM04-GM14-GM04-GM07 | 158 |
| | ZFP − 55E | GM06-CM03-CM12-GM09-GM01-GM16 | 159 |
| | ZFP − 55F | GM07-CM08-CM05-GM01-GM09-GM06 | 160 |
| | ZFP − 55G | GM13-CM11-CM11-GM06-GM02-GM06 | 161 |
| | ZFP − 55H | GM04-CM14-CM05-GM09-GM09-GM13 | 162 |
| | ZFP − 55I | GM07-CM04-CM15-GM02-GM12-GM09 | 163 |
| | ZFP − 55J | GM06-CM13-CM04-GM16-GM09-GM13 | 164 |
| +113 | ZFP + 113A | CM13-GM07-GM09-GM03-GM02-GM06 | 166 |
| ATGGCCTTCCTCTCCTGC | ZFP + 113B | CM06-GM13-GM12-GM02-GM03-GM12 | 167 |
| (SEQ ID NO: 165) | ZFP + 113C | CM11-GM03-GM09-GM03-GM08-GM06 | 168 |
| | ZFP + 113D | CM06-GM13-GM07-GM01-GM11-GM07 | 169 |
| | ZFP + 113E | CM06-GM07-GM03-GM09-GM07-GM02 | 170 |
| | ZFP + 113F | CM04-GM07-GM02-GM09-GM07-GM02 | 171 |
| | ZFP + 113G | CM14-GM07-GM03-GM09-GM07-GM16 | 172 |
| | ZFP + 113H | CM12-GM13-GM09-GM15-GM02-GM06 | 173 |

^{a}) Shown are TLR4 promoter target sites (named according to their distance to the transcription start site) in column 1.
^{b}) Column 2 names the ZFPs identified in a Y1H screen to bind to TLR4 promoter target sites. The naming scheme is as follows: ZFP followed by the name of the target site and a letter designating different ZFP isolated in the screen.
^{c}) Column 3 shows the constitution of the ZFPs by detailing the individual zinc finger modules according to their established binding preference. GM01 designates a zinc finger module preferably binding to a GAA, GM02 to GCA, GM03 to GGA, GM04 to GTA, GM05 to GAC, GM06 to GCC, GM07 to GGC, GM08 to GTC, GM09 to GAG, GM10 to GCG, GM11 to GGG, GM12 to GTG, GM13 to GAT, GM14 to GCT, GM15 to GGT, GM16 to GTT, and furthermore, CM01 to CAC, CM02 to CAA, CM03 to CAG, CM04 to CAT, CM05 to CCA, CM06 to CCC, CM07 to CCG CM08 to CCT, CM09 to CGA, CM10 to CGC, CM11 to CGG, CM12 to CGT, CM13 to CTA, CM14 to CTG, and CM15 to CTT triplets.
^{d}) Column 4 refers to the sequence IDs of ZFPs identified to bind to the respective target site sequence.

TABLE 4
Target sites within FCER1A promoter and results from Y1H screen

| | ZFPs from | | SEQ |
|---|---|---|---|
| Target site DNA seq. | Y1H screen | ZF modules of isolated ZFPs | ID |
| 5′-3′ ^{a}) | ^{b}) | (F1-F2-F3-F4-F5-F6) ^{c}) | NO^{d}) |
| −147 | ZFP − 147A | GM07-CM14-CM02-GM16-GM15-GM13 | 175 |
| GCCCAGTTGGGCACCATC | ZFP − 147B | GM04-CM14-CM02-GM16-GM16-GM13 | 176 |
| (SEQ ID NO: 174) | ZFP − 147C | GM04-CM14-CM09-GM16-GM16-GM13 | 177 |
| | ZFP − 147D | GM16-CM14-CM09-GM16-GM15-GM13 | 178 |
| | ZFP − 147E | GM15-CM14-CM09-GM16-GM03-GM13 | 179 |
| | ZFP − 147F | GM15-CM09-CM02-GM01-GM15-GM13 | 180 |
| | ZFP − 147G | GM03-CM14-CM09-GM04-GM15-GM13 | 181 |
| +17 | ZFP + 17A | GM15-GM16-GM06-GM13-CM11-GM04 | 183 |
| GTCCATGAAGAAGATGGC | ZFP + 17B | GM14-GM12-GM10-GM13-CM11-GM04 | 184 |
| (SEQ ID NO: 182) | ZFP + 17C | GM12-GM04-GM06-GM13-CM11-GM04 | 185 |
| | ZFP + 17D | GM05-GM11-GM06-GM13-CM11-GM04 | 186 |
| | ZFP + 17E | GM10-GM13-GM11-GM04-CM15-GM04 | 187 |
| | ZFP + 17F | GM05-GM12-GM06-GM13-CM11-GM04 | 188 |
| | ZFP + 17G | GM02-GM12-GM06-GM13-CM03-GM04 | 189 |
| | ZFP + 17H | GM05-GM01-GM01-GM13-CM11-GM04 | 190 |
| | ZFP + 17I | GM02-GM12-GM02-GM13-CM11-GM11 | 191 |

^{a}) Shown are FCER1A promoter target sites (named according to their distance to the transcription start site) in column 1.
^{b}) Column 2 names the ZFPs identified in a Y1H screen to bind to FCER1A promoter target sites. The naming scheme is as follows: ZFP followed by the name of the target site and a letter designating different ZFP isolated in the screen.
^{c}) Column 3 shows the constitution of the ZFPs by detailing the individual zinc finger modules according to their established binding preference. GM01 designates a zinc finger module preferably binding to a GAA, GM02 to GCA, GM03 to GGA, GM04 to GTA, GM05 to GAC, GM06 to GCC, GM07 to GGC, GM08 to GTC, GM09 to GAG, GM10 to GCG, GM11 to GGG, GM12 to GTG, GM13 to GAT, GM14 to GCT, GM15 to GGT, GM16 to GTT, and furthermore, CM01 to CAC, CM02 to CAA, CM03 to CAG, CM04 to CAT, CM05 to CCA, CM06 to CCC, CM07 to CCG CM08 to CCT, CM09 to CGA, CM10 to CGC, CM11 to CGG, CM12 to CGT, CM13 to CTA, CM14 to CTG, and CM15 to CTT triplets.
^{d}) Column 4 refers to the sequence IDs of ZFPs identified to bind to the respective target site sequence.

The artificial transcription factors according to the invention comprise a zinc finger protein based on the zinc finger module composition shown in Tables 1 to 4, columns 3, where up to three individual zinc finger modules are exchanged against other zinc finger modules with alternative binding characteristic to modulate the binding of the artificial transcription factor to its target sequence.

The artificial transcription factors according to the invention comprise a zinc finger protein based on the zinc finger module composition shown in Tables 1 to 4, columns 3, where individual amino acids are exchanged in order to minimize potential immunogenicity while retaining binding affinity to the intended target site.

Preferably, the artificial transcription factors according to the invention comprise a zinc finger protein of a protein sequence selected from the group consisting of SEQ ID NO: 31 to SEQ ID NO: 37, SEQ ID NO: 39 to SEQ ID NO: 43, SEQ ID NO: 45 to SEQ ID NO: 50, SEQ ID NO: 52, SEQ ID NO: 54 to SEQ ID NO: 57, SEQ ID NO: 59 to SEQ ID NO: 64, SEQ ID NO: 66 to SEQ ID NO: 80, SEQ ID NO: 82 to SEQ ID NO: 95, SEQ ID NO: 97 to SEQ ID NO: 118, SEQ ID NO: 120 to SEQ ID NO: 136, SEQ ID NO: 138 to SEQ ID NO: 143, SEQ ID NO: 145 to SEQ ID NO: 153, SEQ ID NO: 155 to SEQ ID NO: 164, SEQ ID NO: 166 to SEQ ID NO: 173, SEQ ID NO: 175 to SEQ ID NO: 181, and SEQ ID NO: 183 to SEQ ID NO: 191.

More preferably, the artificial transcription factors according to the invention comprise a pentameric zinc finger protein of SEQ ID NO 135 or a hexameric zinc finger protein of a protein sequence selected from the group consisting of SEQ ID NO 33, 54, 56, 64, 68, 83, 84, 85, 97, 101, 114, 118, 122, 127, 133, 140, 142, 146, 147, 156, 159, 169, 171, 173, 175, 181, 184, 187, 189, and 191.

Even more preferred are the artificial transcription factors comprising a pentameric zinc finger protein of SEQ ID NO 135 or a hexameric zinc finger protein of a protein sequence selected from the group consisting of SEQ ID NO 56, 83, 85, 101, 114, 118, 127, 133, 140, 142, 146, 147, 156, 159, 175, and 181.

Even more preferred are the artificial transcription factors comprising hexameric zinc finger proteins of SEQ ID NO 118, 127, 146, 156, or 175.

Even more preferred are the artificial transcription factors comprising hexameric zinc finger proteins of SEQ ID NO 118, 127, 156, or 175.

Most preferred are the artificial transcription factors comprising hexameric zinc finger proteins of SEQ ID NO 118, 156, or 175.

The polydactyl zinc finger proteins are fused to a regulatory domain, which is either an inhibitory or an activatory protein domain. Inhibitory protein domains considered are the transcriptionally active domains of proteins defined by gene ontology GO:0001071 such as N-terminal KRAB, C-terminal KRAB, SID and ERD domains, preferably KRAB or SID. Activatory protein domains considered are the transcriptionally active domains of proteins defined by gene ontology GO:0001071 such as VP16 or VP64 (tetrameric repeat of VP16), preferably VP64.

Further to the polydactyl zinc finger proteins fused to an inhibitory or activatory protein domain, the artificial transcription factors of the invention comprise a nuclear localization sequence (NLS). Nuclear localization sequences considered are amino acid motifs conferring nuclear import through binding to proteins defined by gene ontology GO:0008139, for example clusters of basic amino acids containing a lysine residue followed by a lysine or arginine residue, followed by any amino acid, followed by a lysine or arginine residue (K-K/R-X-K/R consensus sequence, Chelsky D. et al., 1989 Mol Cell Biol 9, 2487-2492) or the SV40 NLS, with the SV40 NLS being preferred.

The artificial transcription factors of the invention further comprise optionally a protein transduction domain (PTD). Protein transduction domains considered are the HIV derived TAT peptide, the HSV-1 VP22 peptide, the synthetic peptide mT02 (PVRRPRRRRRRK, SEQ ID NO: 192, Yoshikawa T. et al. 2009 Biomaterials 30, 3318-23), the synthetic peptide mT03 (THRLPRRRRRRK, SEQ ID NO: 193), the R9 peptide (RRRRRRRRR, SEQ ID NO: 194), the ANTP domain, and the protective antigen/lethal factor N terminus PTD, preferably the TAT PTD.

Artificial transcription factors directed to a receptor gene promoter, but without the protein transduction domain, are also a subject of the invention. They are intermediates for the artificial transcription factors of the invention as defined hereinbefore.

Artificial transcription factors directed to ETRA, but without the protein transduction domain, are also a subject of the invention. They are intermediates for the artificial transcription factors of the invention as defined hereinbefore.

Artificial transcription factors directed to the ETRB promoter, but without the protein transduction domain, are also a subject of the invention. They are intermediates for the artificial transcription factors of the invention as defined hereinbefore.

Artificial transcription factors directed to the TLR4 promoter, but without the protein transduction domain, are also a subject of the invention. They are intermediates for the artificial transcription factors of the invention as defined hereinbefore.

Artificial transcription factors directed to the FCER1A promoter, but without the protein transduction domain, are also a subject of the invention. They are intermediates for the artificial transcription factors of the invention as defined hereinbefore.

The domains of the artificial transcription factors of the invention may be connected by short flexible linkers. A short flexible linker has 2 to 8 amino acids, preferably glycine and serine. A particular linker considered is GGSGGS (SEQ ID NO: 9). Artificial transcription factors may further contain markers to ease their detection and processing.

### Activity of Artificial Transcription Factors in Regulating Receptor Promoter Activity

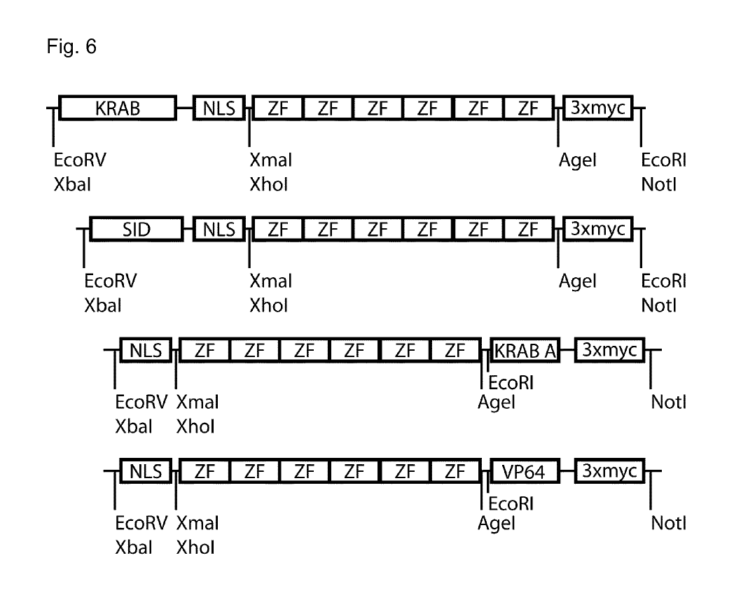

Zinc finger module based artificial transcription factors were constructed according to the scheme shown in FIG. 6 from ZFPs (see Tables 1 to 4) selected using Y1H screening to specifically bind to certain target sites of receptor promoters. These artificial transcription factors contained different transcriptionally active domains such as N-terminal KRAB, C-terminal KRAB, SID or VP64. Based on published data (Beerli R. R. et al., 1998 Proc Natl Acad Sci USA 95, 14628-14633), KRAB as well as SID domains are predicted to act as transcriptional repressors, while VP64 mediates transcriptional activation. To assess the potential of artificial transcription factors (the fusion between a 6ZFP and a transcriptionally active domain) to influence transcription driven by the receptor promoter, a luciferase reporter assay was employed. To this end, cells capable of driving expression from a certain promoter were co-transfected with an artificial transcription factor expression plasmid together with a dual-reporter plasmid. The dual-reporter plasmid contained the secreted Gaussia luciferase gene under the control of the receptor promoter in question together with the gene for secreted alkaline phosphatase (SEAP) under control of the constitutive CMV promoter based on the NEG-PG04 and EF1a-PG04 plasmids (GeneCopoeia, Rockville, Md.).

Many promoters display cell type-specific expression patterns with virtually no expression in some cell types and high level expression in other cell types. Thus, the selection of a suitable cell model for promoter regulation studies depends on the tissue-specificity of a given receptor promoter. In the instances shown here, HeLa cells, a cervix carcinoma cell line, are able to express the luciferase reporter from ETRA, ETRB or TLR4 promoter. Expression of luciferase under control of the FCER1A promoter was not possible in HeLa cells due to the tissue-specificity of this promoter. Therefore, rat basophilic leukemia RBL-2H3 cells were employed to assess artificial transcription factor function against the FCER1A promoter. This cell line supported expression of the FCER1A promoter-driven luciferase reporter and was transfectable with around 50% efficiency using nucleofection.

This co-transfection was done in a 3:1 ATF:reporter plasmid ratio to ensure the presence of artificial transcription factor (ATF) expression in cells transfected with the reporter plasmid and luciferase, and SEAP activity was measured according to manufacturer's recommendation (GeneCopoeia, Rockville, Md.). Luciferase values were normalized to SEAP activity and compared to control cells expressing yellow fluorescent protein (YFP) set to 100%. By measuring the ratio between luciferase and SEAP activity in the supernatant of transfected cells, normalization of receptor promoter-driven luciferase expression to SEAP expression only in cells transfected with artificial transcription factor plasmid was possible. This approach proved useful to account and normalize for differences in transfection efficiency between different experiments and allowed for quantification of artificial transcription factor mediated regulation of a given receptor promoter.

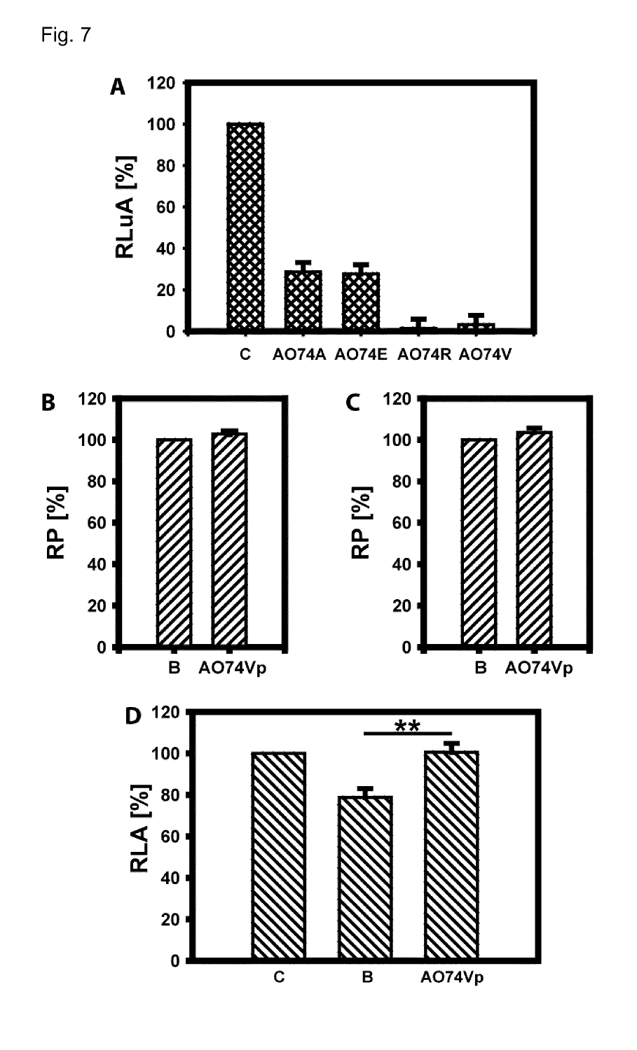

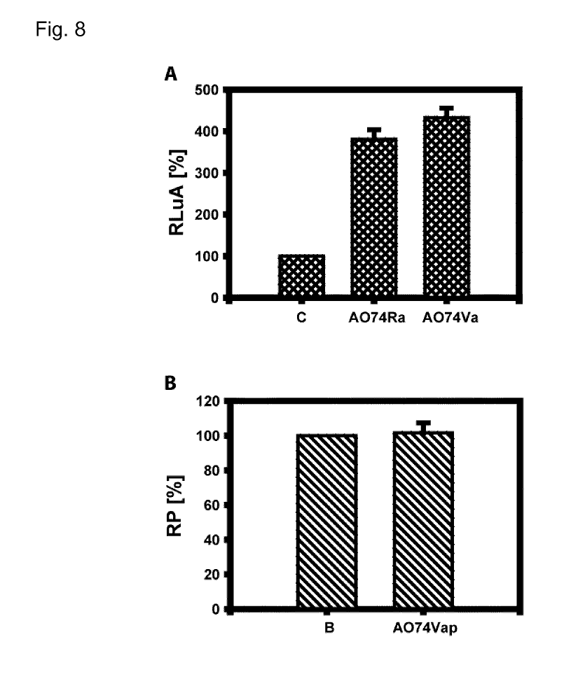

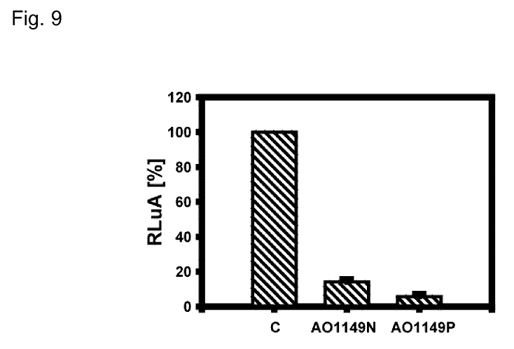

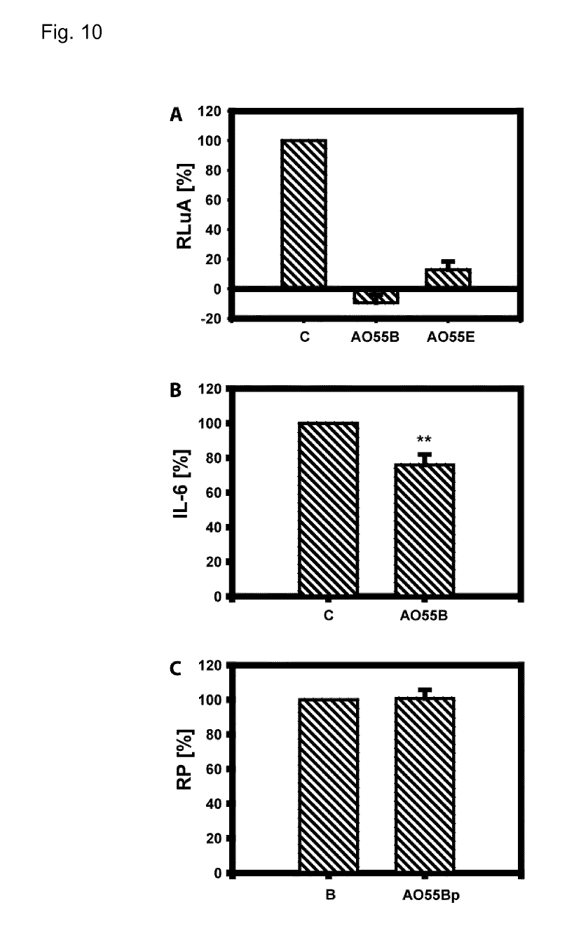

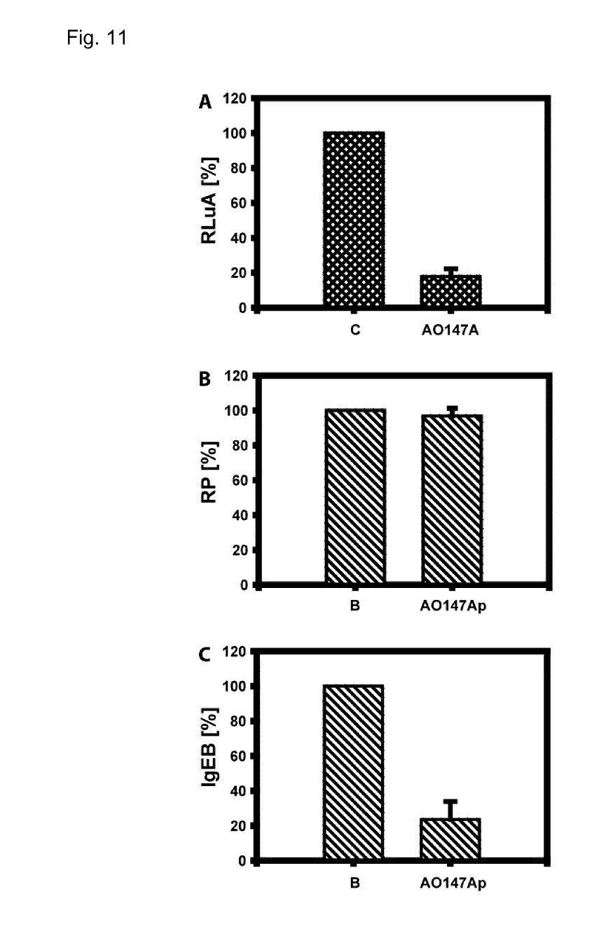

All luciferase expression studies (FIGS. 7A to 11A) were performed at least three times in triplicates, averaged, compared to control transfected cells, expressed as relative luciferase acivity (RLuA) in % of control and plotted with error bars depicting SEM.

The naming convention for artificial transcription factors in is as follows: Artificial transcription factors expressed in mammalian cells using a mammalian expression vector and consisting of a zinc finger protein (ZFP), a nuclear localization sequence and a negatively regulatory domain such as SID or KRAB (N- or C-terminal) are designated with the letters AO followed by a number representing a target site and a letter identifying a certain ZFP identified using Y1H screening. The addition of a lower case “a” to this name designates an artificial transcription factor containing the activatory VP64 domain. The addition of a lower case “p” designates a purified artificial transcription factor protein produced in a heterologous expression system and in addition to aforementioned domains containing the protein transduction domain TAT and the HA tag (SEQ ID NO: 195).

FIG. 7A shows the artificial transcription factor-dependent downregulation of ETRA promoter-dependent luciferase expression. HeLa cells were co-transfected with an ETRA promoter luciferase/constitutive SEAP reporter construct as described above and expression plasmids for AO74A, AO74E, AO74R, AO74V or yellow fluorescent protein (YFP) as control (labeled C). These artificial transcription factors are directed against TS+74 of ETRA promoter and contain the negative regulatory SID domain. While AO74A and AO74E suppressed ETRA promoter-driven expression by about 70%, AO74R, and AO74V were capable of blocking the ETRA promoter to background levels.

FIG. 8A highlights the versatility of the approach for generating transcription factors targeting receptor promoters. By simply exchanging the inhibitory domain SID in AO74V or AO74R against the activatory domain VP64, activating transcription factors capable of boosting transcriptional activity of the ETRA promoter to around 400% could be generated.

Using the same approach, artificial transcription factors targeting the ETRB receptor promoter were constructed. FIG. 9A shows repression of ETRB promoter activity by AO1149N and AO1149P containing a ZFP directed against target site TS−1149 of the ETRB promoter (see FIG. 2) as well as an inhibitory SID domain. HeLa cells were co-transfected with an ETRB promoter luciferase/SEAP reporter construct and expression plasmids for AO1149N, AO1149P or YFP as control. AO1149N suppressed ETRB promoter activity by around 80%, while AO1149P blocked the ETRB promoter almost to background levels.

To analyze the activity of TLR4-specific artificial transcription factors AO55B and AO55E consisting of a ZFP directed against target site TS−55 in the TLR4 promoter (see FIG. 3) and the inhibitor KRAB domain at the C-terminus, the Gaussia luciferase/SEAP reporter assay was employed. As shown in FIG. 10, expression of AO55B or AO55E in HeLa cells repressed TLR4 promoter driven expression with AO55B, completely blocking luciferase expression compared to control transfected cells expressing YFP.

For assessing activity of artificial transcription factors directed against the FCER1A promoter, AO147A was expressed in rat basophilic RBL-2H3 cells together with FCER1A promoter driven Gaussia luciferase and CMV-driven SEAP as above. This artificial transcription factor is directed against target site TS−147 and contains a N-terminal KRAB domain. RBL-2H3 cells were chosen based on the tissue-specificity of the FCER1A promoter and the ease of transfection using nucleoporation. As shown in FIG. 11, FCER1A-driven expression in RBL-2H3 cells producing AO147A is reduced by around 80% in comparison to YFP expressing control cells (C).

Taken together, artificial transcription factor mediated regulation of receptor promoter-driven regulation is feasible and is able to upregulate expression by up to 400% or completely block expression depending on the regulatory domain used for artificial transcription factor construction opening up a possible regulatory range in the order of almost two orders of magnitude.

### Assessing Potential Toxic Effects of Artificial Transcription Factors

Although artificial transcription factors are selected for a given target site and although the target sites chosen were unique inside the human genome, artificial transcription factors might have off-target effects by binding to similar sequences thereby exerting toxic effects. Such toxic effects might potentially interfere with functional assays of such artificial transcription factors. For any given unique 18 bp target site any number of highly similar sequences can be identified with one, two or three substitutions. While these sequences might allow binding of an artificial transcription factor and might lead to off-target effects, most such off-target sites are located in other locations than in the regulatory sequences of actively transcribed genes, greatly ameliorating the potential for off-target effects of artificial transcription factor treatment. For the following experiments, cells were treated with 1 μM of transducible artificial transcription factor protein and cellular proliferation as measure of potential toxicity was assessed using MTS assay. Each experiment was performed in triplicates at least three times; proliferation of artificial transcription factor treated cells was averaged and expressed as percent of control treated with the corresponding buffer. Of note, the addition of “p” to the artificial transcription factor designation indicates a protein containing TAT protein transduction domain, zinc finger proteins and a regulatory domain such as SID, KRAB or VP64 rather than an expression plasmid coding for artificial transcription factors without protein transduction domain.

As shown in FIG. 7B, treatment of HeLa cells with 1 μM of AO74Vp protein, the transducible version of AO74V, for two days did not result in the loss of proliferation compared to cells treated with buffer. Similarly, treatment of hUtSMCs with AO74Vp was not toxic to these cells and did not inhibit their proliferation (FIG. 7C). In addition, the ZFP−74V based activating artificial transcription factor protein AO74Vap did not display any toxic effects on hUtSMC proliferation (FIG. 8B). These data are consistent with negligible off-target binding of ZFP−74V containing artificial transcription factors in the human genome. Similarly, neither treatment of HeLa cells with the TLR4-specific artificial transcription factor AO55 Bp (FIG. 10C) or the FCER1A-specific AO147Ap (FIG. 11B) did result in loss of proliferation. Taken together, all tested artificial transcription factors are highly specific towards their intended target site and do not cause off-target effects that might result in cellular death or a decrease in proliferation.

### Functional Analysis of Artificial Transcription Factors Using Cellular Response Assays

To ascertain artificial transcription factor function on endogenous receptor promoters following TAT-based protein delivery, the cellular response to receptor agonist treatment between artificial transcription factor and control treated cells was observed.

### Assessment of ETRA Downregulation Following Artificial Transcription Factor Treatment

Smooth muscle cells (SMCs) express ETRA and are capable of contraction following exposure to ET-1. To measure the effectiveness of anti-ETRA promoter artificial transcription factor AO74V, human uterine smooth muscle cells (hUtSMCs) were used as model system. To this end, hUtSMCs were embedded into 3-dimensional collagen lattices and treated for three days with 1 μM AO74Vp or buffer control before exposure to 0 or 100 nM ET-1. The protein or buffer treatment was repeated every 24 hours. Following detachment of the lattices from their support and addition of ET-1, contraction of lattices was observed. As shown in FIG. 7D, control lattices exposed to ET-1 contract to about 78% compared to lattices not treated with ET-1. In contrast, AO74V treated lattices did not significantly contract in the presence of ET-1 when compared to control lattices not treated with ET-1. This is consistent with a complete block of ET-1 induced contraction of hUtSMCs following treatment with AO74Vp. The data shown in FIG. 7D represents the average lattice area 9 hours after ET-1 addition of three independent experiments done in sextuplicates. Statistical analysis using the SPSS software package employing a general linear univariate model revealed high significance (** represent p<0.001) for the blocking action of AO74Vp.

### Assessment of TLR4 Downregulation Following Artificial Transcription Factor Treatment

Macrophages express TLR4 and produce in response to LPS binding to TLR4 pro-inflammatory cytokines such as IL-6. Phorbol 12-myristate 13-acetate (PMA)-stimulated U937 cells are a widely accepted model for human macrophage-like cells. To measure the effectiveness of anti-TLR4 promoter artificial transcription factor AO55B, PMA-stimulated U937 cells expressing AO55B or YFP as control were challenged for 8 hours with 0.5 ng/ml LPS and the production of IL-6 was measured using ELISA. As shown in FIG. 10B, expression of AO55B significantly reduced (p<0.005) the secretion of IL-6 compared to control cells by around 25%. Taken into account that U937 nucleofection efficiency is about 50%, meaning AO55B was in these experiments only expressed in about 50% of the cells, the actual repression of IL-6 production by AO55B is in the order of 50%.

Assessment of FCER1 Function Following Artificial Transcription Factor Treatment

Binding of an IgE antibody to the heterotrimeric high-affinity IgE receptor FCER1 on the surface of macrophages, mast cells and basophiles is the first step in triggering an allergic response in an atopic individual. Encounter with an allergen leads to the cross-linking of IgE-loaded FCER1 molecules triggering an intra-cellular signaling cascade resulting in the release of allergic mediators and cytokines. Thus the ability of IgE to bind to e.g. basophiles is one crucial step in the allergic process. To assess the IgE bindability following treatment with an artificial transcription factor directed against the promoter of the alpha subunit of the FCER1, human basophilic KU812F cells were treated daily for 48 hours with 1 μM of AO147Ap or buffer. Following treatment, IgE bindability was measured using flow cytometry. The average IgE bindability (IgEB) of AO147A treated KU812F cells of three independent experiments is shown in FIG. 110. Treatment with AO147Ap reduced IgE bindability of basophilic cells by about 80% compared to control treated cells. Interestingly, although artificial transcription factor AO147Ap is directed only against the alpha subunit of the FCER1 receptor complex, the function of the whole receptor in terms of ability to bind IgE is greatly reduced. Thus, allergen-induced FCER1 cross-linking is expected to be reduced greatly, increasing the threshold for allergic mediator release. Unlike some other receptors, FCER1 is a multimeric protein complex comprised of alpha, beta, and gamma subunits encoded by three different genetic loci. Only a correctly assembled FCER1 containing one alpha, one beta and two gamma chains, with the alpha chain providing the IgE binding site, is able to trigger allergic responses. Thus, downregulating the expression of the FCER1 alpha chain (FCER1A) e.g. with a suitable artificial transcription factor will prevent the correct assembly and function of the FCER1 as a whole. This notion is supported by the FCER1A−/− mouse where anaphylaxis is abolished (Dombrowicz D., 1993, Cell 75, 969-976). Thus, targeting FCER1A expression using artificial transcription factor technology is suitable to abrogate allergic reactions. Furthermore, although artificial transcription factors are highly specific for one target gene, multimeric receptors in general are amenable to artificial transcription factor mediated knock-down.

### Pharmaceutical Compositions

The present invention relates also to pharmaceutical compositions comprising an artificial transcription factor as defined above. Pharmaceutical compositions considered are compositions for parenteral systemic administration, in particular intravenous administration, compositions for inhalation, and compositions for local administration, in particular ophthalmic-topical administration, e.g. as eye drops, or intravitreal, subconjunctival, parabulbar or retrobulbar administration, to warm-blooded animals, especially humans. Particularly preferred are eye drops and compositions for intravitreal, subconjunctival, parabulbar or retrobulbar administration. The compositions comprise the active ingredient alone or, preferably, together with a pharmaceutically acceptable carrier. Further considered are slow-release formulations. The dosage of the active ingredient depends upon the disease to be treated and upon the species, its age, weight, and individual condition, the individual pharmacokinetic data, and the mode of administration.

Further considered are pharmaceutical compositions useful for oral delivery, in particular compositions comprising suitably encapsulated active ingredient, or otherwise protected against degradation in the gut. For example, such pharmaceutical compositions may contain a membrane permeability enhancing agent, a protease enzyme inhibitor, and be enveloped by an enteric coating.

The pharmaceutical compositions comprise from approximately 1% to approximately 95% active ingredient. Unit dose forms are, for example, ampoules, vials, inhalers, eye drops and the like.

The pharmaceutical compositions of the present invention are prepared in a manner known per se, for example by means of conventional mixing, dissolving or lyophilizing processes.

Preference is given to the use of solutions of the active ingredient, and also suspensions or dispersions, especially isotonic aqueous solutions, dispersions or suspensions which, for example in the case of lyophilized compositions comprising the active ingredient alone or together with a carrier, for example mannitol, can be made up before use. The pharmaceutical compositions may be sterilized and/or may comprise excipients, for example preservatives, stabilizers, wetting agents and/or emulsifiers, solubilizers, salts for regulating osmotic pressure and/or buffers and are prepared in a manner known per se, for example by means of conventional dissolving and lyophilizing processes. The said solutions or suspensions may comprise viscosity-increasing agents, typically sodium carboxymethylcellulose, carboxymethylcellulose, dextran, polyvinylpyrrolidone, or gelatins, or also solubilizers, e.g. Tween 80® (polyoxyethylene(20)sorbitan mono-oleate).

Suspensions in oil comprise as the oil component the vegetable, synthetic, or semi-synthetic oils customary for injection purposes. In respect of such, special mention may be made of liquid fatty acid esters that contain as the acid component a long-chained fatty acid having from 8 to 22, especially from 12 to 22, carbon atoms. The alcohol component of these fatty acid esters has a maximum of 6 carbon atoms and is a monovalent or polyvalent, for example a mono-, di- or trivalent, alcohol, especially glycol and glycerol. As mixtures of fatty acid esters, vegetable oils such as cottonseed oil, almond oil, olive oil, castor oil, sesame oil, soybean oil and groundnut oil are especially useful.

The manufacture of injectable preparations is usually carried out under sterile conditions, as is the filling, for example, into ampoules or vials, and the sealing of the containers.

For parenteral administration, aqueous solutions of the active ingredient in water-soluble form, for example of a water-soluble salt, or aqueous injection suspensions that contain viscosity-increasing substances, for example sodium carboxymethylcellulose, sorbitol and/or dextran, and, if desired, stabilizers, are especially suitable. The active ingredient, optionally together with excipients, can also be in the form of a lyophilizate and can be made into a solution before parenteral administration by the addition of suitable solvents.

Compositions for inhalation can be administered in aerosol form, as sprays, mist or in form of drops. Aerosols are prepared from solutions or suspensions that can be delivered with a metered-dose inhaler or nebulizer, i.e. a device that delivers a specific amount of medication to the airways or lungs using a suitable propellant, e.g. dichlorodifluoro-methane, trichlorofluoromethane, dichlorotetrafluoroethane, carbon dioxide or other suitable gas, in the form of a short burst of aerosolized medicine that is inhaled by the patient. It is also possible to provide powder sprays for inhalation with a suitable powder base such as lactose or starch.

Eye drops are preferably isotonic aqueous solutions of the active ingredient comprising suitable agents to render the composition isotonic with lacrimal fluid (295-305 mOsm/l). Agents considered are sodium chloride, citric acid, glycerol, sorbitol, mannitol, ethylene glycol, propylene glycol, dextrose, and the like. Furthermore the composition comprise buffering agents, for example phosphate buffer, phosphate-citrate buffer, or Tris buffer (tris(hydroxymethyl)-aminomethane) in order to maintain the pH between 5 and 8, preferably 7.0 to 7.4. The compositions may further contain antimicrobial preservatives, for example parabens, quaternary ammonium salts, such as benzalkonium chloride, polyhexamethylene biguanidine (PHMB) and the like. The eye drops may further contain xanthan gum to produce gel-like eye drops, and/or other viscosity enhancing agents, such as hyaluronic acid, methylcellulose, polyvinylalcohol, or polyvinylpyrrolidone.

### Use of Artificial Transcription Factors in a Method of Treatment

Furthermore the invention relates an artificial transcription factors directed to the endothelin receptor A promoter as described above for use in influencing the cellular response to endothelin, for lowering or increasing endothelin receptor levels, and for use in the treatment of diseases modulated by endothelin, in particular for use in the treatment of such eye diseases. Likewise the invention relates to a method of treating a disease modulated by endothelin comprising administering a therapeutically effective amount of an artificial transcription factor directed to the endothelin receptor A promoter to a patient in need thereof.

Diseases modulated by endothelin are, for example, cardiovascular diseases such as essential hypertension, pulmonary hypertension, chronic heart failure but also chronic renal failure. In addition, renal protection before, during and after radioopaque material application is achieved by blunting the endothelin response. In addition, multiple sclerosis is negatively impacted by the endothelin system.

Further diseases modulated by endothelin are diabetic kidney disease or eye diseases such as glaucomatous neurodegeneration, vascular dysregulation in ocular blood circulation, retinal vein occlusion, retinal artery occlusion, macular edema, age related macula degeneration, optic neuropathy, central serous chorioretinopathy, retinitis pigmentosa, Susac syndrome, and Leber's hereditary optic neuropathy.

Likewise the invention relates to a method of treating a disease modulated by endothelin comprising administering a therapeutically effective amount of an artificial transcription factor of the invention to a patient in need thereof. In particular the invention relates to a method of treating glaucomatous neurodegeneration, vascular dysregulation in ocular blood circulation, in particular to a method of treating retinal vein occlusion, retinal artery occlusion, macular edema, optic neuropathy, central serous chorioretinopathy, retinitis pigmentosa, and Leber's hereditary optic neuropathy, comprising administering an effective amount of an artificial transcription factor of the invention to a patient in need thereof. The effective amount of an artificial transcription factor of the invention depends upon the particular type of disease to be treated and upon the species, its age, weight, and individual condition, the individual pharmacokinetic data, and the mode of administration. For administration into the eye, a monthly vitreous injection of 0.5 to 1 mg is preferred. For systemic application, a monthly injection of 10 mg/kg is preferred. In addition, implantation of slow release deposits into the vitreous of the eye is also preferred.

Furthermore the invention relates to an artificial transcription factor directed to the endothelin receptor B promoter as described above for use in influencing the cellular response to endothelin, for lowering or increasing endothelin receptor B levels, and for use in the treatment of diseases modulated by endothelin, in particular for use in the treatment of such eye diseases. Likewise the invention relates to a method of treating a disease modulated by endothelin comprising administering a therapeutically effective amount of an artificial transcription factor directed to the endothelin receptor B promoter to a patient in need thereof.

Diseases modulated by ET-1-dependent, ETRB-mediated artificial transcription factors are certain cancers, neurodegeneration and inflammation-related disorders.

Furthermore the invention relates to an artificial transcription factor directed to the TLR4 promoter as described above for use in influencing the cellular response to LPS, for lowering or increasing TLR4 levels, and for use in the treatment of diseases modulated by LPS, in particular for use in the treatment of such eye diseases. Likewise the invention relates to a method of treating a disease modulated by LPS comprising administering a therapeutically effective amount of an artificial transcription factor directed to the TLR4 promoter to a patient in need thereof. Diseases modulated by LPS are rheumatoid arthritis, artherosclerosis, psoriasis, Crohn's disease, uveitis, contact lens associated keratitis, corneal inflammation, resistance of cancers to chemotherapy and the like.

Furthermore the invention relates to an artificial transcription factor directed to the FCER1A promoter as described above for use in influencing the cellular response to IgE or IgE-antigen complexes, for lowering or increasing FCER1 levels, and for use in the treatment of diseases modulated by IgE or IgE-antigen complexes, in particular for use in the treatment of such eye diseases.

Likewise the invention relates to a method of treating a disease modulated by IgE or IgE-antigen complexes comprising administering a therapeutically effective amount of an artificial transcription factor directed to the FCER1A promoter to a patient in need thereof. Diseases modulated by IgE or IgE-antigen complexes are allergic rhinitis, asthma, eczema and anaphylaxis and the like.

### Use of Artificial Transcription Factors in Plants

Furthermore the invention relates to the use of artificial transcription factors targeting plant receptors. Preferably, DNA encoding the artificial transcription factors is cloned into vectors for transformation of plant-colonizing microorganisms or plants. Alternatively, the artificial transcription factors are directly applied in suitable compositions for topical applications to plants.

### EXAMPLES

### Cloning of DNA Plasmids

For all cloning steps, restriction endonucleases and T4 DNA ligase were purchased from New England Biolabs. Shrimp Alkaline Phosphatase (SAP) was from Promega. The high-fidelity Platinum Pfx DNA polymerase (Invitrogen) was applied in all standard PCR reactions. DNA fragments and plasmids were isolated according to the manufacturer's instructions using NucleoSpin Extract II kit, NucleoSpin Plasmid kit, or NucleoBond Xtra Midi Plus kit (Macherey-Nagel). Oligonucleotides were purchased from Sigma-Aldrich. All relevant DNA sequences of newly generated plasmids were verified by sequencing (Microsynth).

### Design and Cloning of Two Hexameric Zinc Finger Proteins (ZFP−855a and ZFP+74a)

To generate artificial transcription factors regulating ETRA expression, a fusion protein consisting of TAT-KRAB-ZFP was designed and the corresponding, codon-optimized DNA sequence was obtained through gene synthesis. For the ZFP part of the fusion protein, human ETRA promoter region (−1000 bp to +100 bp relative to the transcription start site; RefSeq DNA NG_{—}013343) was screened for potential (GNN)₆ 6ZFP target sites using the ZiFiT software (Sander J. D. et al., 2010, Nucleic Acids Res 38, W462-468; 2007, Nucleic Acids Res 35, W599-605) with parameters set to “modular assembly” using the so called “Barbas modules” set. For ZFP−855A intended to bind target site −855 (starting at −855 bp relative to transcription start site) ZF59-ZF59-ZF72-ZF58-ZF71-ZF67 was constructed according to Wright D. A. et al., 2006, Nat Protoc 1, 1637-1652. Similarly, ZF65-ZF62-ZF58-ZF65-ZF59-ZF59 was assembled for ZFP+74A intended to bind to target site +74 (+74 bp relative to transcription start site).

As repressor domain for the artificial transcription factor, the KRAB domain consisting of amino acids 1-97 of human KOX1 protein was chosen (Beerli, R. R. et al., 1998, Proc Natl Acad Sci USA 95, 14628-14633). For nuclear targeting, amino acids PKKKRKV (SEQ ID NO: 196) (corresponding to the SV40 NLS) as well as YKDDDDK (SEQ ID NO: 197) (FLAG tag) were incorporated into the fusion protein. Synthetic genes coding for XhoI-NcoI-KRAB-NLS-FLAG-SpeI-ZFP−855A-HindIII and SpeI-ZFP+74A-HindIII (to replace ZFP−855A with ZFP+74A) were codon optimized and synthesized by GenScript. KRAB-NLS-FLAG-ZFP−855A and KRAB-NLS-FLAG-ZFP+74A were inserted into pcDNA3(−) (Invitrogen) after cutting both inserts and vector with XhoI/HindIII, resulting in pAN1021 and pAN1022, respectively.

### Cloning of Hexameric Zinc Finger Protein Libraries for Prey Plasmids

Several hexameric zinc finger protein libraries containing GNN and/or CNN binding zinc finger (ZF) modules were cloned according to Gonzalez B. et al., 2010, Nat Protoc 5, 791-810, with the following improvements. DNA sequences coding for GNN and CNN ZF modules were synthesized and inserted into pUC57 (GenScript) resulting in pAN1049 and pAN1073, respectively. Stepwise assembly of ZFP libraries was done in pBluescript SK (+) vector. To avoid insertion of multiple ZF modules during each individual cloning step leading to non-functional proteins, pBluescript (and its derived products containing 1ZFP, 2ZFPs, or 3ZFPs) and pAN1049 or pAN1073 were first incubated with one restriction enzyme and afterwards treated with SAP. Enzymes were removed by NucleoSpin Extract II kit before the second restriction endonuclease was added.

Cloning of pBluescript-1ZFPL was done by treating 5 μg pBluescript with XhoI, SAP and subsequently SpeI. Inserts were generated by incubating 10 μg pAN1049 (release of 16 different GNN ZF modules) or pAN1073 (release of 15 different CNN ZF modules) with SpeI, SAP and subsequently XhoI. For generation of pBluescript-2ZFPL and pBluescript-3ZFPL, 7 μg pBluescript-1ZFPL or pBluescript-2ZFPL were cut with AgeI, dephosphorylated, and cut with SpeI. Inserts were obtained by applying SpeI, SAP, and subsequently XmaI to 10 μg pAN1049 or pAN1073, respectively. Cloning of pBluescript-6ZFPL was done by treating 6 μg of pBluescript-3ZFPL with AgeI, SAP, and thereafter SpeI to obtain cut vectors. 3ZFPL inserts were released from the pBluescript-3ZFPL by incubating with SpeI, SAP, and subsequently XmaI.

Ligation reactions for libraries containing one, two, and three ZFPs were set up in a 3:1 molar ratio of insert:vector using 200 ng cut vector, 400 U T4 DNA ligase in 20 μl total volume at RT (room temperature) overnight. Ligation reactions of hexameric zinc finger protein libraries included 2000 ng pBluescript-3ZFPL, 500 ng 3ZFPL insert, 4000 U T4 DNA ligase in 200 μl total volume, which were divided into ten times 20 μl and incubated separately at RT over night. Portions of ligation reactions were transformed into Escherichia coli by several methods depending on the number of clones required for each library. For generation of pBluescript-1ZFPL and pBluescript-2ZFPL, 3 μl of ligation reaction were directly used for heat shock transformation of E. coli NEB 5-alpha. Ligation reactions of pBluescript-3ZFPL were desalted by dialyzing for 1 h against DNA-grade H₂O using 0.05 μm VMWP filters (Millipore) before transformation into electrocompetent E. coli NEB 5-alpha (EasyjecT Plus electroporator from EquiBio, 2.5 kV and 25 μF, 2 mm electroporation cuvettes from Bio-Rad). Ligation reactions of pBluescript-6ZFP libraries were applied to NucleoSpin Extract II kit and DNA was eluted in 15 μl of deionized water. About 60 ng of desalted DNA were mixed with 50 μl NEB 10-beta electrocompetent E. coli (New England Biolabs) and electroporation was performed as recommended by the manufacturer using EasyjecT Plus, 2.5 kV, 25 μF and 2 mm electroporation cuvettes.

Multiple electroporations were performed for each library and cells were directly pooled afterwards to increase library size. After heat shock transformation or electroporation, SOC medium was applied to the bacteria and after 1 h of incubation at 37° C. and 250 rpm, 30 μl of SOC culture were used for serial dilutions and plating on LB plates containing ampicillin. The next day, total number of obtained library clones was determined. In addition, ten clones of each library were chosen to isolate plasmid DNA and to check incorporation of inserts by restriction enzyme digestion. At least three of these plasmids were sequenced to verify diversity of the library. The remaining SOC culture was transferred to 100 ml LB medium containing ampicillin and cultured over night at 37° C. and 250 rpm. Those cells were used to prepare plasmid Midi DNA for each library.

For yeast one hybrid screens, hexameric zinc finger protein libraries were transferred to a compatible prey vector. For that purpose, the multiple cloning site of pGAD10 (Clontech) was modified by cutting the vector with XhoI/EcoRI and inserting annealed oligonucleotides OAN971 (TCGACAGGCCCAGGCGGCCCTCGAGGATATCATGATG ACTAGTGGCCAGGCCGGCCC, SEQ ID NO: 198) and OAN972 (AATTGGGCCGGC CTGGCCACTAGTCATCATGATATCCTCGAGGGCCGCCTGGGCCTG, SEQ ID NO: 199). The resulting vector pAN1025 was cut and dephosphorylated, 6ZFP library inserts were released from pBluescript-6ZFPL by XhoI/SpeI. Ligation reactions and electroporations into NEB 10-beta electrocompetent E. coli were done as described above for pBluescript-6ZFP libraries.

For yeast one hybrid screens with increased sensitivity, 6ZFP libraries were transferred into another compatible prey vector. For that purpose, a 1460 bp SphI fragment of pAN1025 was ligated into pAN1373, a modified pRS315 (Sikorski, R. S. and Hieter, P., 1989, Genetics 122(1), 19-27) where an ApaI, NarI fragment was replaced by annealed oligonucleotides OAN1143 (CGCCGCATGCATTCATGCAGGCC, SEQ ID NO: 200) and OAN1144 (TGCATGAATGCATGCGG, SEQ ID NO: 201). This modification resulted in a yeast one hybrid vector containing a low copy ARS/CEN compared to a high copy 2μ origin of replication compatible with the library cloning scheme outlined above.

### Cloning of Receptor Promoter Region for Combined Secreted Luciferase and Alkaline Phosphatase Assay

DNA fragments containing promoter regions of ETRA, ETRB, TLR4 or FCER1A were cloned into pAN1485 (NEG-PG04, GeneCopeia) or pAN1486 (EF1a-PG04, GeneCopeia) resulting in reporter plasmids containing secreted Gaussia luciferase under the control of a receptor promoter and secreted alkaline phosphatase under the control of the constitutive CMV promoter allowing for normalization of luciferase to alkaline phosphatase signal. In detail, ETRA promoter was amplified from human genomic DNA using OAN981 (AATCGCGAGCTCCTTAAGAAACTGGCAGCTTCCACTT, SEQ ID NO: 202) and OAN982 (AATCGCCTCGAGCTGCCGGGTCCGCGCGGCG, SEQ ID NO: 203) and cloned SacI/XhoI into pBluescript resulting in pAN1031. ETRA promoter was cut from pAN1031 using XhoI/Klenow/BamHI and cloned into pAN1486 cut HindIII/Klenow/BglII resulting in pAN1492. ETRB promoter was amplified from human genomic DNA using OAN1232 (GCTAGCTGTCGACACATGGTGCGTGATAACTTGCCC, SEQ ID NO: 204) and OAN1233 (GCTAGCTGGTACCAGGCCTGCTGCTACCTGCTCCAGAAGGC, SEQ ID NO: 205) and cloned SacI/KpnI into pBluescript resulting in pAN1432. ETRB promoter was cut from pAN1432 StuI/EcoRI and cloned into pAN1486 cut with HindIII/Klenow/EcoRI resulting in pAN1489. TLR4 promoter was amplified from human genomic DNA using OAN1234 (GCTAGCTGTCGACATAAGCCAGTGACAAAAAGAT ACATAC, SEQ ID NO: 206) and OAN1235 (GCTAGCTGGTACCAGGCCTTATTTGAT CTCTGTGGCTTCTTGAG, SEQ ID NO: 207) and cloned SalI/KpnI into pBluescript resulting in pAN1433. TLR4 promoter fragment was cut from pAN1433 StuI/BamHI and cloned into pAN1486 HindIII/Klenow/BglII resulting in pAN1491. TLR4 promoter was amplified from pAN1491 using OAN1249 (CTAGCTGATATCAGCTTAGCGGTTTAC ATGACTTGAC, SEQ ID NO: 208) and OAN1250 (CTAGCTAAGCTTCACGCAGGA GAGGAAGGCCATG, SEQ ID NO: 209) and cloned EcoRV/HindIII into pAN1486 resulting in pAN1509. FCER1A promoter was amplified from human genomic DNA using OAN1236 (GCTAGCTGTCGACTTAAATTCCTATTTATTAACCTTTTTAGC, SEQ ID NO: 210) and OAN1237 (GCTAGCTGGTACCAGGCCTGTCACCACCCACAGTAAAGGTTC, SEQ ID NO: 211) and cloned SacI/KpnI into pBluescript resulting in pAN1434. FCER1A promoter was cut from pAN1434 StuI/EcoRI and cloned into pAN1486 HindIII/Klenow/EcoRI resulting in pAN1490. FCER1A promoter was amplified from pAN1490 using OAN1261 (CTAGCTGAT ATCGCTAGCCATGCTCCTGAATATGTAT, SEQ ID NO: 212) and OAN1262 (CTAGCTAAGCTTGGCAGGAGCCCTCTTCTTCATGGACTCCTGG, SEQ ID NO: 213) and cloned EcoRV/HindIII into pAN1485 resulting in pAN1515.

### Cloning of Bait Plasmids

For each bait plasmid, a 18 bp target site flanked by 21 bps taken from the sequence upstream and downstream in the ETRA, ETRB, TLR4 or FCER1A promoter region were used. A NcoI site was included for restriction analysis. Oligonucleotides were designed and annealed in such a way to produce 5′ HindIII and 3′ XhoI sites which allowed direct ligation into pAbAi (Clontech,) cut with HindIII/XhoI (Table 5).

TABLE 5
Oligonucleotides used for cloning of target sites into pAbAi vector

| | | | |
|---|---|---|---|
| Target | | | bait |
| site | Oligo name | Oligo sequence 5′-3′ | plasmid |

ETRA

| | | | |
|---|---|---|---|
| −855 | OAN1018 | AGCTTGTGAACTGTCTTGGAAGTGGATCCTCCA | pAN1083 |
| | | GCCCCTGCTACATGGAGCAAAAACGAGCTGTC | |
| | | CCATGGC (SEQ ID NO: 214), 60 bp insert | |
| | OAN1019 | TCGAGCCATGGGACAGCTCGTTTTTGCTCCAT | |
| | | GTAGCAGGGGCTGGAGGATCCACTTCCAAGAC | |
| | | AGTTCACA (SEQ ID NO: 215), 60 bp insert | |
| −555 | OAN1082 | AGCTTAGGCAGTGGCCTTTGTCCCTCATCTCCT | pAN1160 |
| | | CTCCCACCCCCAATTTAGGATAAAGTATCTGCC | |
| | | CATGGC (SEQ ID NO: 216) | |
| | OAN1083 | TCGAGCCATGGGCAGATACTTTATCCTAAATTG | |
| | | GGGGTGGGAGAGGAGATGAGGGACAAAGGCC | |
| | | ACTGCCTA (SEQ ID NO: 217) | |
| −487 | OAN1084 | AGCTTAGACGTTGAGACCCACTTTCTGTAAGGT | pAN1161 |
| | | CGGCTTCTTCATTGTTTGAATTTCTTGAGGTTC | |
| | | CATGGC (SEQ ID NO: 218) | |
| | OAN1085 | TCGAGCCATGGAACCTCAAGAAATTCAAACAAT | |
| | | GAAGAAGCCGACCTTACAGAAAGTGGGTCTCA | |
| | | ACGTCTA (SEQ ID NO: 219) | |
| −447 | OAN1090 | AGCTTATTGTTTGAATTTCTTGAGGTTTCACGG | pAN1164 |
| | | AGCCACGCGCTGGAACCTTCCATAGTCTCTCC | |
| | | CCATGGC (SEQ ID NO: 220) | |
| | OAN1091 | TCGAGCCATGGGGAGAGACTATGGAAGGTTCC | |
| | | AGCGCGTGGCTCCGTGAAACCTCAAGAAATTC | |
| | | AAACAATA (SEQ ID NO: 221) | |
| −37 | OAN1092 | AGCTTAAAAAAGACTCCTGCCCTTCAGGGCCT | pAN1165 |
| | | GGAAGGGGGCGGCAGCTTTGTGCTTTTTAGTG | |
| | | GCCATGGC (SEQ ID NO: 222) | |
| | OAN1093 | TCGAGCCATGGCCACTAAAAAGCACAAAGCTG | |
| | | CCGCCCCCTTCCAGGCCCTGAAGGGCAGGAG | |
| | | TCTTTTTTA (SEQ ID NO: 223) | |
| −306 | OAN1024 | AGCTTGCGTGCTCCCTCTTAAGTTTAGAGGCC | pAN1086 |
| | | GTTGAGGAGCCGAAGTGGACAGCAGTTTACTG | |
| | | GCCATGGC (SEQ ID NO: 224) | |
| | OAN1025 | TCGAGCCATGGCCAGTAAACTGCTGTCCACTT | |
| | | CGGCTCCTCAACGGCCTCTAAACTTAAGAGGG | |
| | | AGCACGCA (SEQ ID NO: 225) | |
| −230 | OAN1088 | AGCTTGGCAGGGAAGACGGAGAAGAAACCACC | pAN1163 |
| | | CGTGGGCCCTGGCTCTGTGTCCAGTTGTTCCG | |
| | | TCCATGGC (SEQ ID NO: 226) | |
| | OAN1089 | TCGAGCCATGGACGGAACAACTGGACACAGAG | |
| | | CCAGGGCCCACGGGTGGTTTCTTCTCCGTCTT | |
| | | CCCTGCCA (SEQ ID NO: 227) | |
| −103 | OAN1022 | AGCTTGTCTGTCAAACTCTACCCTCTCTCCTCC | pAN1085 |
| | | ACATCCCCCACCTTTTCTTTCAGGAAGGAAATC | |
| | | CATGGC (SEQ ID NO: 228) | |
| | OAN1023 | TCGAGCCATGGATTTCCTTCCTGAAAGAAAAGG | |
| | | TGGGGGATGTGGAGGAGAGAGGGTAGAGTTT | |
| | | GACAGACA (SEQ ID NO: 229) | |
| +74 | OAN1020 | AGCTTAGTGGAAGGTCTGGAGCTTTGGGAGGA | pAN1084 |
| | | GACGGGGAGGACAGACTGGAGGCGTGTTCCT | |
| | | CCCCATGGC (SEQ ID NO: 230) | |
| | OAN1021 | TCGAGCCATGGGGAGGAACACGCCTCCAGTCT | |
| | | GTCCTCCCCGTCTCCTCCCAAAGCTCCAGACC | |
| | | TTCCACTA (SEQ ID NO: 231) | |

ETRB

| | | | |
|---|---|---|---|
| −1149 | OAN1198 | AGCTTGGACGAGGACTGCCCCCCTCCCTCGG | pAN1383 |
| | | GCAACTACTACTGATGCTGTCCAGGCATCGCC | |
| | | CACCATGGC (SEQ ID NO: 232) | |
| | OAN1199 | TCGAGCCATGGTGGGCGATGCCTGGACAGCAT | |
| | | CAGTAGTAGTTGCCCGAGGGAGGGGGGCAGT | |
| | | CCTCGTCCA (SEQ ID NO: 233) | |
| −487 | OAN1214 | AGCTTCGAGTTCAATCGCGGGGTATAGAGGTT | pAN1417 |
| | | CCCCTGCGGGGCAAAATGCAGAGCTTGACACA | |
| | | ACCATGGC (SEQ ID NO: 234) | |
| | OAN1215 | TCGAGCCATGGTTGTGTCAAGCTCTGCATTTTG | |
| | | CCCCGCAGGGGAACCTCTATACCCCGCGATTG | |
| | | AACTCGA (SEQ ID NO: 235) | |

TLR4

| | | | |
|---|---|---|---|
| −276 | OAN1186 | AGCTTACCTGATTGTTTTCCTAAATTCACCAAG | pAN1377 |
| | | CCCAGGCAGAGGTCAGATGACTAATTGGGATA | |
| | | CCATGGC (SEQ ID NO: 236) | |
| | OAN1187 | TCGAGCCATGGTATCCCAATTAGTCATCTGACC | |
| | | TCTGCCTGGGCTTGGTGAATTTAGGAAAACAAT | |
| | | CAGGTA (SEQ ID NO: 237) | |
| −55 | OAN1188 | AGCTTACTGCTTTGAATACACCAATTGCTGTGG | pAN1378 |
| | | GGCGGCTCGAGGAAGAGAAGACACCAGTGCC | |
| | | TCCATGGC (SEQ ID NO: 238) | |
| | OAN1189 | TCGAGCCATGGAGGCACTGGTGTCTTCTCTTC | |
| | | CTCGAGCCGCCCCACAGCAATTGGTGTATTCA | |
| | | AAGCAGTA (SEQ ID NO: 239) | |
| +113 | OAN1190 | AGCTTGCTGGGACTCTGATCCCAGCCATGGCC | pAN1379 |
| | | TTCCTCTCCTGCGTGAGACCAGAAAGCTGGGA | |
| | | GCCATGGC (SEQ ID NO: 240) | |
| | OAN1191 | TCGAGCCATGGCTCCCAGCTTTCTGGTCTCAC | |
| | | GCAGGAGAGGAAGGCCATGGCTGGGATCAGA | |
| | | GTCCCAGCA (SEQ ID NO: 241) | |

FCER1A

| | | | |
|---|---|---|---|
| −147 | OAN1192 | AGCTTTAAGTGGGTAAATATTAAATTGCCCAGT | pAN1380 |
| | | TGGGCACCATCCTGAATATTATCTCTAAAGAAC | |
| | | CATGGC (SEQ ID NO: 242) | |
| | OAN1193 | TCGAGCCATGGTTCTTTAGAGATAATATTCAGG | |
| | | ATGGTGCCCAACTGGGCAATTTAATATTTACCC | |
| | | ACTTAA (SEQ ID NO: 243) | |
| +17 | OAN1194 | AGCTTCCAGCACAGTAAGCACCAGGAGTCCAT | pAN1381 |
| | | GAAGAAGATGGCTCCTGCCATGGAATCCCCTA | |
| | | CCCATGGC (SEQ ID NO: 244) | |
| | OAN1195 | TCGAGCCATGGGTAGGGGATTCCATGGCAGGA | |
| | | GCCATCTTCTTCATGGACTCCTGGTGCTTACTG | |
| | | TGCTGGA (SEQ ID NO: 245) | |

### Cloning of Artificial Transcription Factors for Mammalian Transfection

For generation of DNA fragment XbaI-EcoRV-NNNNNN-XhoI-NNNNNN-AgeI-3xmyc-STOP-NotI-EcoRI, 3xmyc tag was amplified from pWS250 with Platinum Pfx DNA polymerase, OAN1032 (AATCGCTCTAGAGATATCATATATCTCGAGATATATACCGGT GAGCAGAAACTCATCTCTG, SEQ ID NO: 246), and OAN1033 (GCGATTGAATTCGC GGCCGCTTACAGATCTTCCTCAGAGA, SEQ ID NO: 247), cut with XbaI/EcoRI, ligated into pcDNA3(−) cut with XbaI/EcoRI, resulting in pAN1109. KRAB-NLS was amplified from pAN1021 using Platinum Pfx DNA polymerase, OAN1034 (AATCGCGATATCATGGATG CTAAGTCCCTGA, SEQ ID NO: 248), and OAN1035 (GCGATTCTCGAGCCCCACTTTA CGTTTCTTTT, SEQ ID NO: 249). The PCR product was cut with EcoRV/XhoI and ligated into pAN1109 cut with EcoRV/XhoI resulting in pAN1110.

DNA sequence of ZFP−855A was amplified from pAN1021 with Platinum Pfx DNA polymerase, OAN1036 (AATCGCCTCGAGCCCGGGCCGGGTGAAAAGCCCTAT, SEQ ID NO: 250), OAN1037 (GCGATTACCGGTCTGTGCTGATGAGCCCC, SEQ ID NO: 251), digested with XhoI/AgeI and cloned into pAN1110 cut with XhoI/AgeI to produce pAN1111. Similarly, ZFP+74A was amplified from pAN1022 with OAN1038 (AATCGCCTC GAGCCCGGGCCAGGCGAAAAGCCCTAC, SEQ ID NO: 252) and OAN1039 (GCGATTA CCGGTCTGTGCTGAACTACCGCC, SEQ ID NO: 253), cloned into pAN1110 and resulting in pAN1112.

ZFP−855A of pAN1111 was replaced by appropriate 6ZFPs (identified by yeast one hybrid screen) using XhoI/AgeI digestion, e.g. by ZFP−855C resulting in pAN1133.

In addition, SID-NLS (SID corresponds to amino acids 1-36 of Mad mSin3 interaction domain according to Beerli, R. R. et al., 1998, Proc Natl Acad Sci USA 95, 14628-14633) was generated by annealing OAN1096 (AATCGCGATATCATGGCGGCGGCGGTTCGG ATGAACATCCAGATGCTGCTGGA, SEQ ID NO: 254), OAN1097 (ATCCAGATGCTGCT GGAGGCGGCCGACTATCTGGAGCGGCGGGAGAGAGAAGCT, SEQ ID NO: 255), OAN1098 (GGTATGGTAACATGGAGGCATAACCATGTTCAGCTTCTCTCTCCCGC, SEQ ID NO: 256), OAN1099 (GCGATTCTCGAGCCCCACTTTACGTTTCTTTTTCGGGT ATGGTAACATGGAGG, SEQ ID NO: 257) in a first DNA synthesis step using Platinum Pfx DNA polymerase. An aliquot of this PCR product was used as template for the second DNA synthesis step with Platinum Pfx DNA polymerase, OAN1096, and OAN1099. The second PCR product was cut with XhoI/EcoRV and used to replace KRAB-NLS in pAN1111 cut with XhoI/EcoRV. The resulting plasmid pAN1208 was used to replace ZFP−855A with any 6ZFP from Y1H screens after via XhoI/AgeI treatment.

Furthermore, the order of protein domains was rearranged according to Gommans, W. M. et al., 2007, Mol Carcinog 46, 391-401 with a N-terminal NLS followed by 6ZFP, GGSGGS (SEQ ID NO: 9) linker sequence, amino acids 11-55 of human KRAB and C-terminal 3xmyc tag. First, DNA fragment AgeI-EcoRI-NNNNNN-BamHI-3xmyc-STOP-NotI-HindIII was generated by PCR with pAN1133 as template, Platinum Pfx DNA polymerase, OAN1100 (GCGATTACCGGTGAATTCATATATGGATCCGAGCAGAAA CTCATCTCT, SEQ ID NO: 258), OAN1101 (GCGATTAAGCTTGCGGCCGCTTACAG ATCTTCCTCAGAGA, SEQ ID NO: 259), cut with AgeI/HindIII, ligated into pAN1109 cut with AgeI/HindIII producing pAN1183. Second, EcoRV-ATG-NLS-XhoI-XmaI-ZFP−855C-AgeI-GGSGGS linker-EcoRI was created by PCR with pAN1133 as template, Platinum Pfx DNA polymerase, OAN1104 (GCGATTGATATC ATGCCGAAAAAGAAACGTAAAG, SEQ ID NO: 260), OAN1105 (GCGATTGAATTCGCTGCCGCCGCTGCCGCCACCGG TATGAGTCCTCT, SEQ ID NO: 261) and inserted into pAN1183 using EcoRI/EcoRV cloning to produce pAN1184. Third, amino acids 11-55 of human KRAB were amplified from pAN1133 with Platinum Pfx DNA polymerase, OAN1106 (GCGATTGAATTCC GCACACTGGTTACCT, SEQ ID NO: 262), OAN1107 (GCGATTGGATCCATAGCC CAGGCTAACC, SEQ ID NO: 263), cut with EcoRI/BamHI and ligated into pAN1184 cut with EcoRI/BamHI. The final plasmid pAN1185 was used to replace ZFP−855C with any 6ZFP from Y1H screens by cutting with XhoI/AgeI.

For cloning of activating ATFs the C-terminal KRAB domain was replaced by VP64 coding sequence by cutting with EcoRI/BamHI and inserting annealed OAN1253 (SEQ ID NO: 264), OAN1254 (SEQ ID NO: 265), OAN1255 (SEQ ID NO: 266) and OAN1256 (SEQ ID NO: 267).

### Modified Yeast One Hybrid (Y1H) Screen

### Yeast Strain and Media

Saccharomyces cerevisiae Y1H Gold was purchased from Clontech, YPD medium and YPD agar from Carl Roth. Synthetic drop-out (SD) medium contained 20 g/l glucose, 6.8 g/l Na₂HPO₄.2H₂O, 9.7 g/l NaH₂PO₄.2H₂O (all from Carl Roth), 1.4 g/l yeast synthetic drop-out medium supplements, 6.7 g/l yeast nitrogen base, 0.1 g/l L-tryptophan, 0.1 g/l L-leucine, 0.05 g/l L-adenine, 0.05 g/l L-histidine, 0.05 g/l uracil (all from Sigma-Aldrich). SD-U medium contained all components except uracil, SD-L was prepared without L-leucine. SD agar plates did not contain sodium phosphate, but 16 g/l Bacto Agar (BD). Aureobasidin A (AbA) was purchased from Clontech.

### Preparation of Bait Yeast Strains

About 5 μg of each bait plasmid were linearized with BstBI in a total volume of 20 μl and half of the reaction mix was directly used for heat shock transformation of S. cerevisiae Y1H Gold. Yeast cells were used to inoculate 5 ml YPD medium the day before transformation and grown over night on a roller at RT. One milliliter of this pre-culture was diluted 1:20 with fresh YPD medium and incubated at 30° C., 225 rpm for 2-3 h. For each transformation reaction 1 OD₆₀₀ was harvested by centrifugation, yeast cells were washed once with 1 ml sterile water and once with 1 ml TE/LiAc (10 mM Tris/HCl, pH 7.5, 1 mM EDTA, 100 mM lithium acetate). Finally, yeast cells were resuspended in 50 μl TE/LiAc and mixed with 50 μg single stranded DNA from salmon testes (Sigma-Aldrich), 10 ul of BstBI-linearized bait plasmid (see above), and 300 μl PEG/TE/LiAc (10 mM Tris/HCl, pH 7.5, 1 mM EDTA, 100 mM lithium acetate, 50% (w/v) PEG 3350). Cells and DNA were incubated on a roller for 20 min at RT, afterwards placed into a 42° C. water bath for 15 min. Finally, yeast cells were collected by centrifugation, resuspended in 100 μl sterile water and spread onto SD-U agar plates. After 3 days of incubation at 30° C. eight clones growing on SD-U from each transformation reaction were chosen to analyze their sensitivity towards Aureobasidin A (AbA). Pre-cultures were grown over night on a roller at RT. For each culture, OD₆₀₀ was measured and OD₆₀₀=0.3 was adjusted with sterile water. From this first dilution five additional 1/10 dilution steps were prepared with sterile water. For each clone 5 μl from each dilution step were spotted onto agar plates containing SD-U, SD-U 100 ng/ml AbA, SD-U 150 ng/ml AbA, and SD-U 200 ng/ml AbA. After incubation for 3 days at 30° C., three clones growing well on SD-U and being most sensitive to AbA were chosen for further analysis. Stable integration of bait plasmid into yeast genome was verified by Matchmaker Insert Check PCR Mix 1 (Clontech) according to the manufacturer's instructions. One of three clones was used for subsequent Y1H screen.

Transformation of Bait Yeast Strain with Hexameric Zinc Finger Protein Library

50 μl of yeast bait strain pre-culture were diluted into 100 ml YPD medium and incubated at 30° C. and 225 rpm until OD₆₀₀=1.6-2.0 (circa 20 h). Cells were collected by centrifugation in a swing-out rotor (5 min, 1500×g, 4° C.). Preparation of electrocompetent cells was done according to Benatuil L. et al., 2010, Protein Eng Des Sel 23, 155-159. For each transformation reaction, 400 μl electrocompetent bait yeast cells were mixed with 1 μg prey plasmids encoding 6ZFP libraries and incubated on ice for 3 min. Cell-DNA suspension was transferred to a pre-chilled 2 mm electroporation cuvette. After electroporation (EasyjecT Plus electroporator, 2.5 kV and 25 μF) yeast cells were transferred to 8 ml of 1:1 mix of YPD:1 M Sorbitol and incubated at 30° C. and 225 rpm for 90 min. Cells were collected by centrifugation and resuspended in 1 ml of SD-L medium. Aliquots of 50 μl were spread on 10 cm SD-L agar plates containing 1000-4000 ng/ml AbA. In addition, 50 μl of cell suspension were used to make 1/100 and 1/1000 dilutions and 50 μl of undiluted and diluted cells were plated on SD-L. All plates were incubated at 30° C. for 3 days. The total number of obtained clones was calculated from plates with diluted transformants. While SD-L plates with undiluted cells indicate growth of all transformants, AbA-containing SD-L plates only resulted in colony formation if the prey 6ZFP bound to its bait target site successfully.

### Verification of Positive Interactions and Recovery of 6ZFP-Encoding Prey Plasmids

For initial analysis, forty good-sized colonies were picked from SD-L plates containing the highest AbA concentration and yeast cells were restreaked twice on SD-L with 3000-4000 ng/ml AbA to obtain single colonies. For each clone, one colony was used to inoculate 5 ml SD-L medium and cells were grown at RT over night. The next day, OD₆₀₀=0.3 was adjusted with sterile water, five additional 1/10 dilutions were prepared and 5 μl of each dilution step were spotted onto two plates of SD-L, SD-L 1000 ng/ml AbA, SD-L 1500 ng/ml AbA, SD-L 2000 ng/ml AbA, SD-L 3000 ng/ml AbA, and SD-L 4000 ng/ml AbA. Clones were ranked according to their ability to grow on high AbA concentration. From best growing clones 5 ml of initial SD-L pre-culture were used to spin down cells and to resuspend them in 100 μl water or residual medium. After addition of 50 U lyticase (Sigma-Aldrich, L2524) cells were incubated for 1 h at 30° C. and 300 rpm on a horizontal shaker. Generated spheroblasts were diluted with 250 μl A1 buffer from NucleoSpin Plasmid kit, one spatula tip of glass beads (Sigma-Aldrich, G8772) was added and tubes were mixed vigorously by vortexing for 20 s. Glass beads were allowed to settle and 250 μl of supernatant were transferred to a fresh tube and used to continue with the standard NucleoSpin Plasmid kit protocol. After elution with 50 μl of elution buffer 5 μl of plasmid DNA were transformed into E. coli DH5 alpha by heat shock transformation or electroporation. Two individual colonies were picked from ampicillin-containing LB plates, plasmids were isolated and library inserts were sequenced. Obtained results were analyzed for consensus sequences among the 6ZFPs for each target site.

### Cell Culture and Transfections

HeLa cells were grown in Dulbecco's Modified Eagle's Medium (DMEM) supplemented with 4.5 g/l glucose, 10% heat-inactivated fetal bovine serum, 2 mM L-glutamine, and 1 mM sodium pyruvate (all from Sigma-Aldrich) in 5% CO₂ at 37° C. For luciferase reporter assay, 7000 HeLa cells/well were seeded into 96 well plates. Next day, co-transfections were performed using Effectene Transfection Reagent (Qiagen) according to the manufacturer's instructions. Plasmid midi preparations coding for artificial transcription factor and for luciferase were used in the ratio 3:1. Medium was replaced by 100 μl per well of fresh DMEM 6 h and 24 h after transfection. U937 (Sigma) and KU812F cells (Sigma) were grown in RMPI-1640 media supplemented with 10% FBS, 2 mM glutamine, and 1 mM sodium pyruvate. U937 and KU812F cells were transfected by nucleofection using Cell Line Nucleofector kit C (Amaxa) or Cell Line Nucleofector kit T (Amaxa) according to manufacturer's suggestions. RBL-2H3 cells (DSMZ) were grown in 70% MEM/20% RMPI-1640/10% heat inactivated FBS supplemented with 2 mM glutamine and 1 mM pyruvate. RBL-2H3 cells were nucleofected using Cell line nucleofector kit T (Amaxa).

Primary human uterine smooth muscle cells (hUtSMCs, PromoCell) were grown according to the supplier's suggestion using Smooth Muscle Cell Growth Medium 2.

### Combined Luciferase/SEAP Promoter Activity Assay

HeLa or RBL-2H3 cells were co-transfected with an artificial transcription factor expression construct and a plasmid carrying secreted Gaussia luciferase under the control of the ETRA, ETRB, TLR4 or FCER1 promoter and secreted alkaline phosphatase under the control of the constitutive CMV promoter (Secrete-Pair Dual Luminescence Assay, GeneCopeia, Rockville, Md.). Two days following transfection, cell culture supernatants were collected and luciferase activity and SEAP activity were measured using Secrete-Pair Dual Luminescence assay (GeneCopoeia) or SEAP reporter gene assay (Roche). Co-transfection of YFP—N1 (Clontech) instead of an artificial transcription factor expression construct served as control. Luciferase activity was normalized to SEAP activity and expressed as percentage of control.

Human Uterine Smooth Muscle Cells (hUtSMC) Lattice Contraction Assay

250 μl of sterile bovine collagen (3.1 mg/ml; #5005-B Nutacon) were mixed with 30 μl 10×PBS and 22.5 μl 0.1 N NaOH to reach a pH 7.4. 25000 hUtSMCs in 200 μl of SMC media 2 were added to the neutralized collagen, gently mixed, transferred to 24 well tissue culture plate and allowed to polymerize at 37° C., 5% CO₂ for 45 minutes. After polymerization, 500 μl of SMC growth media 2 were added. For treatment with artificial transcription factor, 1 μM AO74V or an appropriate amount of buffer as control were added right after polymerization and again after 24 and 48 hours. 72 hours after polymerization, lattices were detached from the vessel wall by gently shaking or the help of a spatula and 100 nM of ET-1 or buffer control were added. Lattices were scanned and lattice area was determined by image analysis using ImageJ software.

### IL-6 Detection

1×10⁶ U937 cells were nucleofected with expression plasmids for TLR4-specific artificial transcription factors or control vector according to the manufacturer's recommendation

(Amaxa). 1.25×10⁶ cells from each nucleofection were transferred into 12 well plates and stimulated for 48 hours with 100 nM phorbol-12-myristate-13-acetate (PMA; Sigma) before stimulation with varying LPS concentrations for 8 hours. Concentration of IL-6 was analyzed in cell culture supernatants using IL-6 ELISA (Orgenium) according to the manufacturer's recommendation.

### Flowcytometric Determination of IqE Bindability

To determine binding of IgE to KU812F cells, 1*10⁶ cells were washed once in 2 ml of FACS buffer (1×PBS, 2% FBS) before resuspension in 0.5 ml FACS buffer. 2*10⁵ cells were incubated with 10 μg/ml of human IgE (abcam) for 30 minutes, washed once with 500 μl FACS buffer before addition of FITC-labeled mouse anti-human IgE (5 μg/ml, abcam) for 30 minutes. Samples were washed once in 500 μl FACS buffer and resuspended in 700 μl FACS buffer. Samples were analyzed by flow cytometry (Cyan ADP, Beckman Coulter). Unstained cells and cells only treated with FITC-labeled mouse anti-human IgE were used as controls.

### Determination of Cellular Proliferation Using MTS Assay.

7000 HeLa cells or hUtSMCs were seeded into 96 well plates in 100 μl of media and treated with specific artificial transcription factors or appropriate buffer controls for 48 or 72 hours, respectively. To determine cellular proliferation, the CellTiter 96 Aqueous Non-Radioactive Cell Proliferation Assay (Promega) was used according to the manufacturer's recommendations. Experiments done in triplicates were repeated independently at least three times.

### Production of Artificial Transcription Factor Protein

E. coli BL21(DE3) transformed with expression plasmid for a given artificial transcription factor were grown in 1 L LB media supplemented with 100 μM ZnCl₂ until OD₆₀₀ between 0.8 and 1 was reached, and induced with 1 mM IPTG for two hours. Bacteria were harvested by centrifugation, bacterial lysate was prepared by sonication, and inclusion bodies were purified. To this end, inclusion bodies were collected by centrifugation (5000 g, 4° C., 15 minutes) and washed three times in 20 ml of binding buffer (50 mM HEPES, 500 mM NaCl, 10 mM imidazole; pH 7.5). Purified inclusion bodies were solubilized on ice for one hour in 30 ml of binding buffer A (50 mM HEPES, 500 mM NaCl, 10 mM imidazole, 6M GuHCl; pH 7.5). Solublized inclusion bodies were centrifuged for 40 minutes at 4° C. and 13,000 g and filtered through 0.45 μm PVDF filter. His-tagged artificial transcription factors were purified using His-Trap columns on an Aktaprime FPLC (GeHealthcare) using binding buffer A and elution buffer B (50 mM HEPES, 500 mM NaCl, 500 mM imidazole, 6M GuHCl; pH 7.5). Fractions containing purified artificial transcription factor were pooled and dialyzed at 4° C. overnight against buffer S (50 mM Tris-HCl, 500 mM NaCl, 200 mM arginine, 100 μM ZnCl₂, 5 mM GSH, 0.5 mM GSSG, 50% glycerol; pH 7.5) in case the artificial transcription factor contained a SID domain, or against buffer K (50 mM Tris-HCl, 300 mM NaCl, 500 mM arginine, 100 μM ZnCl₂, 5 mM GSH, 0.5 mM GSSG, 50% glycerol; pH 8.5) for KRAB domain containing artificial transcription factors. Following dialysis, protein samples were centrifuged at 14,000 rpm for 30 minutes at 4° C. and sterile filtered using 0.22 μm Millex-GV filter tips (Millipore).

### Statistical Analysis

Statistical analysis was done employing Student's t-test where appropriate (Excel, Microsoft Cooperation) or a general linear univariate model using SPSS (IBM). All experiments shown are averages of three independent experiments with the error bars representing SEM.

1. An artificial transcription factor comprising a polydactyl zinc finger protein targeting specifically a receptor gene promoter fused to an inhibitory or activatory protein domain, a nuclear localization sequence, and a protein transduction domain.

2. An artificial transcription factor according to claim 1 wherein the receptor gene promoter is the endothelin receptor A promoter.

3. An artificial transcription factor according to claim 1 wherein the receptor gene promoter is the endothelin receptor B promoter.

4. An artificial transcription factor according to claim 1 wherein the receptor gene promoter is the Toll-like receptor 4 promoter.

5. An artificial transcription factor according to claim 1 wherein the receptor gene promoter is the FCER1A promoter.

6. The artificial transcription factor according to claim 1 wherein the zinc finger protein is a hexameric zinc finger protein.

7. The artificial transcription factor according to claim 1 wherein the zinc finger protein is a zinc finger protein of a protein sequence selected from the group consisting of SEQ ID NO: 31 to SEQ ID NO: 37, SEQ ID NO: 39 to SEQ ID NO: 43, SEQ ID NO: 45 to SEQ ID NO: 50, SEQ ID NO: 52, SEQ ID NO: 54 to SEQ ID NO: 57, SEQ ID NO: 59 to SEQ ID NO: 64, SEQ ID NO: 66 to SEQ ID NO: 80, SEQ ID NO: 82 to SEQ ID NO: 95, SEQ ID NO: 97 to SEQ ID NO: 118, SEQ ID NO: 120 to SEQ ID NO: 136, SEQ ID NO: 138 to SEQ ID NO: 143, SEQ ID NO: 145 to SEQ ID NO: 153, SEQ ID NO: 155 to SEQ ID NO: 164, SEQ ID NO: 166 to SEQ ID NO: 173, SEQ ID NO: 175 to SEQ ID NO: 181, and SEQ ID NO: 183 to SEQ ID NO: 191.

8. The artificial transcription factor according to claim 1 wherein the zinc finger protein is a zinc finger protein of a protein sequence selected from the group consisting of SEQ ID NO 56, 83, 85, 101, 114, 118, 127, 133, 140, 142, 146, 147, 156, 159, 175, and 181.

9. The artificial transcription factor according to claim 1 wherein the zinc finger protein is a zinc finger protein of SEQ ID NO 118, 133, 156, or 175.

10. The artificial transcription factor according to claim 1 wherein the zinc finger protein is fused to an inhibitory protein domain.

11. The artificial transcription factor according to claim 10 wherein the inhibitory protein domain is N-terminal KRAB of SEQ ID NO: 1, C-terminal KRAB of SEQ ID NO: 2, SID of SEQ ID NO: 3, or ERD of SEQ ID NO: 4.

12. The artificial transcription factor according to claim 1 wherein the zinc finger protein is fused to an activatory protein domain.

13. The artificial transcription factor according to claim 12 wherein the activatory protein domain is VP16 of SEQ ID NO: 5 or VP64 of SEQ ID NO: 6.

14. The artificial transcription factor according to claim 1 wherein the nuclear localization sequences is a cluster of basic amino acids containing the K-K/R-X-K/R consensus sequence or the SV40 NLS of SEQ ID NO: 196.

15. The artificial transcription factor according to claim 1 wherein the protein transduction domain is the HIV derived TAT peptide of SEQ ID NO: 7, the HSV-1 VP22 peptide, the synthetic peptide mT02 of SEQ ID NO: 192, the synthetic peptide mT03 of SEQ ID NO: 193, the R9 peptide of SEQ ID NO: 194, the ANTP domain, or the protective antigen/lethal factor N terminus PTD.

16. An artificial transcription factor comprising a polydactyl zinc finger protein targeting specifically the endothelin receptor A promoter fused to an inhibitory or activatory protein domain and a nuclear localization sequence.

17. An artificial transcription factor comprising a polydactyl zinc finger protein targeting specifically the endothelin receptor B promoter fused to an inhibitory or activatory protein domain and a nuclear localization sequence.

18. An artificial transcription factor comprising a polydactyl zinc finger protein targeting specifically the Toll-like receptor 4 promoter fused to an inhibitory or activatory protein domain and a nuclear localization sequence.

19. An artificial transcription factor comprising a polydactyl zinc finger protein targeting specifically the FCER1A promoter fused to an inhibitory or activatory protein domain and a nuclear localization sequence.

20. A pharmaceutical composition comprising an artificial transcription factor according to claim 1.

21-26. (canceled)

27. A method of treating diseases comprising administering a therapeutically effective amount of an artificial transcription factor according to claim 1 to a patient in need thereof, wherein the disease to be treated is modulated by the binding of specific effectors to receptors, for which the polydactyl zinc finger protein is specifically targeting the receptor gene promoter.

28. A method of treating a disease modulated by endothelin comprising administering a therapeutically effective amount of an artificial transcription factor according to claim 2 to a patient in need thereof.

29. A method of treating a disease modulated by endothelin comprising administering a therapeutically effective amount of an artificial transcription factor according to claim 3 to a patient in need thereof.

30. A method of treating a disease modulated by lipopolysaccharide comprising administering a therapeutically effective amount of an artificial transcription factor according to claim 4 to a patient in need thereof.

31. A method of treating a disease modulated by IgE comprising administering a therapeutically effective amount of an artificial transcription factor according to claim 5 to a patient in need thereof.


SEQUENCE LISTING (267 sequences)

SEQ ID NO: 1 - protein, 98 aa, Homo sapiens
MDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVS

SEQ ID NO: 2 - protein, 45 aa, Homo sapiens
RTLVTFKDVFVDFTREEWKLLDTAQQIVYRNVMLENYKNLVSLGY

SEQ ID NO: 3 - protein, 36 aa, Homo sapiens
MAAAVRMNIQMLLEAADYLERREREAEHGYASMLPY

SEQ ID NO: 4 - protein, 58 aa, Homo sapiens
GASQCMPLKLRFKRRWSEDCRLEGGGGPAGGFEDEGEDKKVRGEGPGEAGGPLTPRRV

SEQ ID NO: 5 - protein, 13 aa, herpes simplex virus 7
DALDDFDLDMLGS

SEQ ID NO: 6 - protein, 55 aa, Artificial Sequence
  Synthetic contruct
GRADALDDFDLDMLGSDALDDFDLDMLGSDALDDFDLDMLGSDALDDFDLDMLIN

SEQ ID NO: 7 - protein, 11 aa, Human immunodeficiency virus
YGRKKRRQRRR

SEQ ID NO: 8 - protein, 10 aa, Homo sapiens
EQKLISEEDL

SEQ ID NO: 9 - protein, 6 aa, Artificial Sequence
  Synthetic construct
GGSGGS

SEQ ID NO: 10 - protein, 255 aa, Artificial Sequence
  Synthetic construct
MAAAVRMNIQMLLEAADYLERREREAEHGYASMLPYPKKKRKVGLEPGPGEKPYKCPECGKSFSDPGNLVRHQRTHTGEKPYKCPECGKSFSRSDNLVRHQRTHTGEKPYKCPECGKSFSRSDKLVRHQRTHTGEKPYKCPECGKSFSDPGNLVRHQRTHTGEKPYKCPECGKSFSQRAHLERHQRTHTGEKPYKCPECGKSFSQRAHLERHQRTHTGGSSAQTGEQKLISEEDLEQKLISEEDLEQKLISEEDL

SEQ ID NO: 11 - protein, 246 aa, Artificial Sequence
  Synthetic construct
MAAAVRMNIQMLLEAADYLERREREAEHGYASMLPYPKKKRKVGLEPGEKPYKCPECGKSFSRSDELVRHQRTHTGEKPYKCPECGKSFSRSDKLVRHQRTHTGEKPYKCPECGKSFSRSDKLVRHQRTHTGEKPYKCPECGKSFSDPGNLVRHQRTHTGEKPYKCPECGKSFSQRAHLERHQRTHTGEKPYKCPECGKSFSTSGNLVRHQRTHTGEQKLISEEDLEQKLISEEDLEQKLISEEDL

SEQ ID NO: 12 - protein, 246 aa, Artificial Sequence
  Synthetic construct
MAAAVRMNIQMLLEAADYLERREREAEHGYASMLPYPKKKRKVGLEPGEKPYKCPECGKSFSRSDNLVRHQRTHTGEKPYKCPECGKSFSRSDDLVRHQRTHTGEKPYKCPECGKSFSDPGNLVRHQRTHTGEKPYKCPECGKSFSQRAHLERHQRTHTGEKPYKCPECGKSFSDPGHLVRHQRTHTGEKPYKCPECGKSFSDPGHLVRHQRTHTGEQKLISEEDLEQKLISEEDLEQKLISEEDL

SEQ ID NO: 13 - protein, 246 aa, Artificial Sequence
  Synthetic construct
MAAAVRMNIQMLLEAADYLERREREAEHGYASMLPYPKKKRKVGLEPGEKPYKCPECGKSFSRSDNLVRHQRTHTGEKPYKCPECGKSFSRSDELVRHQRTHTGEKPYKCPECGKSFSTSGHLVRHQRTHTGEKPYKCPECGKSFSDPGNLVRHQRTHTGEKPYKCPECGKSFSQSSSLVRHQRTHTGEKPYKCPECGKSFSTSGELVRHQRTHTGEQKLISEEDLEQKLISEEDLEQKLISEEDL

SEQ ID NO: 14 - protein, 279 aa, Artificial Sequence
  Synthetic construct
MHHHHHHGYGRKKRRQRRRGYPYDVPDYAPWDIMAAAVRMNIQMLLEAADYLERREREAEHGYASMLPYPKKKRKVGLEPGEKPYKCPECGKSFSRSDNLVRHQRTHTGEKPYKCPECGKSFSRSDELVRHQRTHTGEKPYKCPECGKSFSTSGHLVRHQRTHTGEKPYKCPECGKSFSDPGNLVRHQRTHTGEKPYKCPECGKSFSQSSSLVRHQRTHTGEKPYKCPECGKSFSTSGELVRHQRTHTGEQKLISEEDLEQKLISEEDLEQKLISEEDL

SEQ ID NO: 15 - protein, 276 aa, Artificial Sequence
  Synthetic construct
MPKKKRKVGLEPGEKPYKCPECGKSFSRSDNLVRHQRTHTGEKPYKCPECGKSFSRSDDLVRHQRTHTGEKPYKCPECGKSFSDPGNLVRHQRTHTGEKPYKCPECGKSFSQRAHLERHQRTHTGEKPYKCPECGKSFSDPGHLVRHQRTHTGEKPYKCPECGKSFSDPGHLVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 16 - protein, 276 aa, Artificial Sequence
  Synthetic construct
MPKKKRKVGLEPGEKPYKCPECGKSFSRSDNLVRHQRTHTGEKPYKCPECGKSFSRSDELVRHQRTHTGEKPYKCPECGKSFSTSGHLVRHQRTHTGEKPYKCPECGKSFSDPGNLVRHQRTHTGEKPYKCPECGKSFSQSSSLVRHQRTHTGEKPYKCPECGKSFSTSGELVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 17 - protein, 309 aa, Artificial Sequence
  Synthetic construct
MHHHHHHGYGRKKRRQRRRGYPYDVPDYAPWDIMPKKKRKVGLEPGEKPYKCPECGKSFSRSDNLVRHQRTHTGEKPYKCPECGKSFSRSDELVRHQRTHTGEKPYKCPECGKSFSTSGHLVRHQRTHTGEKPYKCPECGKSFSDPGNLVRHQRTHTGEKPYKCPECGKSFSQSSSLVRHQRTHTGEKPYKCPECGKSFSTSGELVRHQRTHTGGGSGGSEFGRADALDDFDLDMLGSDALDDFDLDMLGSDALDDFDLDMLGSDALDDFDLDMLINGSEQKLISEEDLEQKLISEEDLEQKLISEEDL

SEQ ID NO: 18 - protein, 246 aa, Artificial Sequence
  Synthetic construct
MAAAVRMNIQMLLEAADYLERREREAEHGYASMLPYPKKKRKVGLEPGEKPYKCPECGKSFSTTGALTEHQRTHTGEKPYKCPECGKSFSTKNSLTEHQRTHTGEKPYKCPECGKSFSRSDKLTEHQRTHTGEKPYKCPECGKSFSRSDKLTEHQRTHTGEKPYKCPECGKSFSRSDNLVRHQRTHTGEKPYKCPECGKSFSRSDKLTEHQRTHTGEQKLISEEDLEQKLISEEDLEQKLISEEDL

SEQ ID NO: 19 - protein, 218 aa, Artificial Sequence
  Synthetic construct
MAAAVRMNIQMLLEAADYLERREREAEHGYASMLPYPKKKRKVGLEPGEKPYKCPECGKSFSQSGHLTEHQRTHTGEKPYKCPECGKSFSRSDKLTEHQRTHTGEKPYKCPECGKSFSRSDKLTEHQRTHTGEKPYKCPECGKSFSRSDNLVRHQRTHTGEKPYKCPECGKSFSRSDKLTEHQRTHTGEQKLISEEDLEQKLISEEDLEQKLISEEDL

SEQ ID NO: 20 - protein, 266 aa, Artificial Sequence
  Synthetic construct
MPKKKRKVGLEPGEKPYKCPECGKSFSQRAHLERHQRTHTGEKPYKCPECGKSFSQSGHLTEHQRTHTGEKPYKCPECGKSFSTSGNLTEHQRTHTGEKPYKCPECGKSFSTSGSLVRHQRTHTGEKPYKCPECGKSFSRSDNLVRHQRTHTGEKPYKCPECGKSFSDPGHLVRHQRTHTGGGSGGSEFRTLVTFKDVFVDFTREEWKLLDTAQQIVYRNVMLENYKNLVSLGYGSEQKLISEEDLEQKLISEEDLEQKLISEEDL

SEQ ID NO: 21 - protein, 266 aa, Artificial Sequence
  Synthetic construct
MPKKKRKVGLEPGEKPYKCPECGKSFSDCRDLARHQRTHTGEKPYKCPECGKSFSRADNLTEHQRTHTGEKPYKCPECGKSFSSRRTCRAHQRTHTGEKPYKCPECGKSFSRSDNLVRHQRTHTGEKPYKCPECGKSFSQSSNLVRHQRTHTGEKPYKCPECGKSFSTSGSLVRHQRTHTGGGSGGSEFRTLVTFKDVFVDFTREEWKLLDTAQQIVYRNVMLENYKNLVSLGYGSEQKLISEEDLEQKLISEEDLEQKLISEEDL

SEQ ID NO: 22 - protein, 299 aa, Artificial Sequence
  Synthetic construct
MHHHHHHGYGRKKRRQRRRGYPYDVPDYAPWDIMPKKKRKVGLEPGEKPYKCPECGKSFSQRAHLERHQRTHTGEKPYKCPECGKSFSQSGHLTEHQRTHTGEKPYKCPECGKSFSTSGNLTEHQRTHTGEKPYKCPECGKSFSTSGSLVRHQRTHTGEKPYKCPECGKSFSRSDNLVRHQRTHTGEKPYKCPECGKSFSDPGHLVRHQRTHTGGGSGGSEFRTLVTFKDVFVDFTREEWKLLDTAQQIVYRNVMLENYKNLVSLGYGSEQKLISEEDLEQKLISEEDLEQKLISEEDL

SEQ ID NO: 23 - protein, 308 aa, Artificial Sequence
  Synthetic construct
MDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVSPKKKRKVGLEPGEKPYKCPECGKSFSDPGHLVRHQRTHTGEKPYKCPECGKSFSRNDALTEHQRTHTGEKPYKCPECGKSFSQSGNLTEHQRTHTGEKPYKCPECGKSFSTSGSLVRHQRTHTGEKPYKCPECGKSFSTSGHLVRHQRTHTGEKPYKCPECGKSFSTSGNLVRHQRTHTGEQKLISEEDLEQKLISEEDLEQKLISEEDL

SEQ ID NO: 24 - protein, 341 aa, Artificial Sequence
  Synthetic construct
MHHHHHHGYGRKKRRQRRRGYPYDVPDYAPWDIMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVSPKKKRKVGLEPGEKPYKCPECGKSFSDPGHLVRHQRTHTGEKPYKCPECGKSFSRNDALTEHQRTHTGEKPYKCPECGKSFSQSGNLTEHQRTHTGEKPYKCPECGKSFSTSGSLVRHQRTHTGEKPYKCPECGKSFSTSGHLVRHQRTHTGEKPYKCPECGKSFSTSGNLVRHQRTHTGEQKLISEEDLEQKLISEEDLEQKLISEEDL

SEQ ID NO: 25 - DNA, 6950 bp, Homo sapiens
gtgtctatgaatttaactattctaggtacctcatctaagtgggataataaaatatctatctttccttttatgtctggcttatttcacttaacataatatcttaaacgttcatccatgtggtagcatatatcattcttttttaaagctgaataatgttctgtgttatgtacatgtatttatatacatatacatacatgtatataccacattttgtttatccattcttccactgatggatatttggattgtttccatcttttggctagttttcaccttttggcttttctgaataatgctgctatgaatatgggtgtacaaatatctgtttgagactctgctttcaattatttaggtaagtaccaaaaagtagaattgctggatcatagggtaattttatgtttaatttttggagggctgccatgctgttttccacaactgctacactatattttacattcagactggcaatgcacaagggttccaatttctcaacattcttgccaacacttgttctttgctgtttttgcttttgttttactataatggctatcctaataggtgtgaaggaagaatttttagtaactagtcctacaccacagtgagatcagctgtctcaataggtgggtcatgatgaatgtgttctagcaaagactggacagattgacatattcagatatgcaggtgatgcactgtccaagtgtgtctggccacagagtgaataagggctgaaatccagcacatgtttcacgggccaagatgtgaactgcctcttttgggaggaagcagtaagtttttctttcccgaaaatattgtcagcttgccaagccacatgcccaaagggtcacctttttttaatataaacaatggcacttataaaagctattagttattctggttggctgattctccctcctagagaagctgtaagattagtgaacagggtaatatctagtctaaccctactagatgactattaaggcctctttcaatggtggttttctgtagatctctttgatggttttacaaaatggtccctaaattctttgacactcctcacactaagggttgggctctatataccctcaccttcaatctgggatttgtgactgcttgactaatagaatcaagcagaaatgacacggtgccagtttctgggcccagcccttaagaaactggcagcttccactttctgtctctggggacattcactcttggatccccgccaccatgctgtgaggaagcccaaaccacaagtctccacaagtctttgtggagagacgcacgtggagaaaaactaacactcaacaccaagcaagtgaactgtcttggaagtggatcctccagcccctgctacatggagcaaaaacgagctgtcccagacaggctctgcccaaactgcagactgataagcagagtaaatgagtgttgttattttaagccactacgttttatagtaatttgtttagctgcagcagatagccggaacagcatgggatataacatgaccagtgctccaacctcacacttctaccatgtgctgactctagcagtgcactgaaggactccaaggcaggccttccctgaggagaccccagtttttactcacatgtcacaggcagtggcctttgtccctcatctcctctcccacccccaatttaggataaagtatctgccgtgataaagacgttgagacccactttctgtaaggtcggcttcttcattgtttgaatttcttgaggtttcacggagccacgcgctggaaccttccatagtctctcctgaggctccttctttgccctgggctggaggtctgtagccgtgggatgctggctacaagggacaagatagaagcaaaccacctgatccagtaaactgctgtccacttcggctcctcaacggcctctaagcttaagagggagcacgcaagccaagcaaaggcggcagggaagacggagaagaaaccacccgtgggccctggctctgtgtccagttgttccgtcacagatcaaatctgcctgcactaagaggatgggttcctctgcaaggcctttcggaattctgagtcttgtctgtcaaactctaccctctctcctccacatcccccaccttttctttcaggaaggaaatagttaaaaaagactcctgcccttcagggcctggaagggggcggcagctttgtgctttttagtggccgcgtcccaggatagctggaaggttaggacgctcttgcggtcccagagtggagtggaaggtctggagctttgggaggagacggggaggacagactggaggcgtgttcctccggagttttctttttcgtgcgagccctcgcgcgcgcgtacagtcatcccgctggtctgacgattgtggagaggcggtggagaggcttcatccatcccacccggtcgtcgccggggattggggtcccagcgagacctccccgggagaagcagtgcccaggaggttttctgaagccggggaagctgtgcagccgaagccgccgccgcgccggagcccgggacaccggccaccctccgcgccacccaccctcgccggctccggcttcctctggcccaggcgccgcgcggacccggcagctgtctgcgcacgccgagctccacggtcggtgcaagtctttcttatcggggactgggactggggcgggtgcggggatggcggagacgctgcctgggcccctcggtcgggagaagacgagagctgggaacgttctggcccgaccgccctgcagcttgggcgacccgtcgcagcaggggctggcacccacttgccccagggcgcgcggggaggcgggcgccttccgtgaggggtgcgctgcggacacgtgtagagttcggggaacttcacctctccacgttagggtttaggattcggagttttaggaagaggaagcccaatagttttccctgggtgaccccttttccccaggcatgcaagaactttgaggaacgccaagctctgtggcttgctccagcgccttcttgtgaagttggctccacagccttttcttactggcttgcttttcctcccccacatctgggtctgggtatgccaagggtagccaagggcactcggtgtgagggactgaggtgtttggggaaaccccctgtgtagctagttttggaaacctgcctgtggaggtctgggcgttttgttctgtgctcgcctttaaaggacacttgttgcttctcacgtgcagaaaacaagggctgcttctagataatcaccctctgtttacatcatcctaatggctttcccctgatagagttgggggtgggaggcatattaaactcaggcgttgtctctaaggagagttgctcatttcttaaccagagagcaaaacctcagaaaacttgtaaaaagttcctacttttacttgttttttcctccacctgtagttgtctatgtgttagctaattgagaaacaccttggcagtcatttcatgtaaattgaatgctctaaactcatgccattttgataaggtctgaatttccaaattaccaattacaggtgaactttgatcctgagagtctcatatgttaaatggttaaagtgtaagcgtaaggattcagtcctctttcttaataatagtttttgcactccccaaactctcagattccaaacaggaagaatgctgaatctatagaccatgtccttcaggtctgaacctgagatgcttcactagcgctatgactgattatgagtgatgattcaaagcacagcctgactcagattcctggaaatgagctacagacccaccttggaagaacctgcagttgaggggcttctttcatttggttttgtacttttcagccaatgataatatgacatccacaggcctttgccaagtatcccttagttttgcagcttaacatgtgtggagagctgatagagaagtcatcagaaatgatttctgtagtggtgtcattgttggaggaggaaggaagggggttttaatgatagctggctctcatgccttaagaacccacagcgttggccttacctcctggcataatcagctaaccaagcagaccagcatgctaacgttcaaagcaccacaggacctctgatgagtagagcagtacccaccgtagccaggtcagttcacaggtcagttcccagccgactgtaaccggcagagagccattccatttctgcaaaggccttctctctctcatttttaagaaagctgttattgaaggttccctttcatttgggaagctggaagacgttagtcacgaaacagcacaagatatgaaagtatttcttctcccccctgtatctgtgatccaaaaaggaggatgacaagaagcttttctcttggagtcctggtgttccctttggaatggaaatggtgtcatttcataggtctatcaatttttggccagatactcactccagattataagaagcaggaaaaaactgcaaactgtttataattaagtctagtcagagctgacaaatacatggcctgcctctagccttgaaaaattgatatttttcccatttaatcagagtgagtcaagtcctataagcaaaattatgtgtcctgccatttcttggctttgaaaccttgacctaaatacttcttcaagactctggtttccttataagtaaaatggagatattgctcctgcctacctagagtcgtaaagctcaaggcagataacagatacatatataataagttactatatgtaaatactattattatttcagagttcagggggaaatctctggggaaggcaaaagtatccaatactcgcactttatatataccctatactttctatgcaacttgaataaatcttattttatccacgtattggccaaacctaagctttactgatttcccaagataattgtcaaaattcctaaaagtggttaacatcaaccttgaatacactcagaaaaaggattcaattttattttttactttttatttatttaatttttttttttttgagacagggtctcactctgctgctcaggatggagtgcagtgactcgatcatagctccctgcagccttaacctcctggactcaagtgattctcccatctcagcctcccaagtagctgagactacaggcacacgccaccatgcctgtctaatcttttttatgttttgtaaagacagggtctcactaagttgcccaggctagtttcaaactcctgggctcaagtgatcctcctgctttggcctcccaaagtgctgagattatagacatgagccaccacacccaaccaggactcaatttttttaagattaaattatgacctgggtatatacatcacagacacgtacacacaccaccgcataaatcagattatgtcttcatttgaagattcataaaagcctacagaaaaggaaatatataaaatattgaaataggatgagctatttttaattatctttgattattccttattaaggtctcctagacttcctggacaggaaggaccacttgctctctgcaagtgacttaaaaataaataaataaataaataaaccacaccagatggtctttgaaaatgtctaaccaccctcctgagtctattgcttcatctaaactctggagacttctttaaaatattttacatacaaataaagcacaagaatgagagacaggatggtttagtggttaacagctccggctcagagttggactgacctgccttagaatcgcagtgttgccacccatcacctgtggccttgacaagctgcttcaccctcctgggcctctgtttccctatcttcaaaattgaaccgtggatctaccccacaggaccattgtatggattacagggcatcatgcacatatagtactgagtacagtgactggcataaaataaatgtagctatataggatggttaaaagatagcatggggacaggtgtcccttggaaaccaaaatactatgactagcctggaaaagttcattactcccatttcattcatcggcaaataccgtattgtgatgataatttctgagaaatgaaaaagcaaaaaaactcctgtaaaaaatttagattttacacattaatcatcaaggttttcaaaacaaatgtttttatcaattatttcattcttaattgaaactaaaattgttagatgtgttaatggtgttaaccagagttctcattaacatttcataacactttagatggctacctgtgctgcacttcatccctatgttcattatttatcatatgcaagacagcacacattttagaagattcaatttttacattcgataattttattttatgcctcaataccctgtaccctttactcagcatataatgccttttctctttcattaaaaaatatgtttgtatctcaactcttgaagatttttgtaattcaggttcttatcagcctgaatgtagagaaagaagtaactactacttaaaggaaaaactgacatagcctcatccatattatgatgtagatataagtatagctatgtatagagatgagtgagagagacaaagctagagagatagggagatggagatagagctagagatggaaatagagacagagatagatagctatagagacggaaatggagacagagacagagacggagatgacagagatgatggagatgatggagatggagatggagactgacatagaggagatagagcttgccctaatagagcttcagtttttttctggttttgctctgtcatagggtaacctgatatacatatatcttatctaatcaccaaatatatcctgcattttaattgaatcaataaataatcattgactatgatctttttggcaactgggttttggaacaaaaattatttttccttttgtttcaggtgaaaaaaaagtgaaggtgtaaaagcagcacaagtgcaataagagatatttcctcaaatttgcctcaagatggaaaccctttgcctcagggc

SEQ ID NO: 26 - DNA, 1284 bp, Homo sapiens
atggaaaccctttgcctcagggcatccttttggctggcactggttggatgtgtaatcagtgataatcctgagagatacagcacaaatctaagcaatcatgtggatgatttcaccacttttcgtggcacagagctcagcttcctggttaccactcatcaacccactaatttggtcctacccagcaatggctcaatgcacaactattgcccacagcagactaaaattacttcagctttcaaatacattaacactgtgatatcttgtactattttcatcgtgggaatggtggggaatgcaactctgctcaggatcatttaccagaacaaatgtatgaggaatggccccaacgcgctgatagccagtcttgcccttggagaccttatctatgtggtcattgatctccctatcaatgtatttaagctgctggctgggcgctggccttttgatcacaatgactttggcgtatttctttgcaagctgttcccctttttgcagaagtcctcggtggggatcaccgtcctcaacctctgcgctcttagtgttgacaggtacagagcagttgcctcctggagtcgtgttcagggaattgggattcctttggtaactgccattgaaattgtctccatctggatcctgtcctttatcctggccattcctgaagcgattggcttcgtcatggtaccctttgaatataggggtgaacagcataaaacctgtatgctcaatgccacatcaaaattcatggagttctaccaagatgtaaaggactggtggctcttcgggttctatttctgtatgcccttggtgtgcactgcgatcttctacaccctcatgacttgtgagatgttgaacagaaggaatggcagcttgagaattgccctcagtgaacatcttaagcagcgtcgagaagtggcaaaaacagttttctgcttggttgtaatttttgctctttgctggttccctcttcatttaagccgtatattgaagaaaactgtgtataacgagatggacaagaaccgatgtgaattacttagtttcttactgctcatggattacatcggtattaacttggcaaccatgaattcatgtataaaccccatagctctgtattttgtgagcaagaaatttaaaaattgtttccagtcatgcctctgctgctgctgttaccagtccaaaagtctgatgacctcggtccccatgaacggaacaagcatccagtggaagaaccacgatcaaaacaaccacaacacagaccggagcagccataaggacagcatgaactga

SEQ ID NO: 27 - DNA, 1180 bp, Homo sapiens
tgtccccggacgaggactgcccccctccctcgggcaactactactgatgctgtccaggcatcgcccaaggggaaaggttgcagcggggtcggaaggcgcgggaggagtctggcggtgattgatgggaagggatgaatgaataaaagtacttgtctgatggcagcagagaccccgagcaaacggtggaggctacactgtctggcattctcgcagcgtttcgtcagagccggacccgcctgcagctcaagggaggcgtgctcctctcccagagcaggctggaacccagctgggttccgcctcccgggaaggtggtctccattcgtcgctctgcatctggtttgtcagatccgagaggtaaacattcgggcttggtgttgaattaaaatcattgattgaaccttattctggggcttcggtttggcttactagtttgggattttaaaaaaataaaaattaagcctatagagagggcaaattaaaattaggttgggtaaaggaaggagcgcgagtgtttgaagccgtttggagggaacagcggtttccaagttcctgctgacttgagaagtctctgcgggtttccgaatctccggcgcactcctgggcgcgctgcgggagctgtagctcagccagccagggagtagcggctttcatccgccgggaggagtctttcgagttcaatcgcggggtatagaggttcccctgcggggcaaaatgcagagcttgacacaagcccttggcctctaggtgccttaattccgcggttcccacgcacgcttaactaagacgtgtctgtattcctcccgttacgtgaaagagttcggagctttgcctgggacccccatcattccctccctggcacaccccttccagaacgccccgccccactgcatattatttacccctcctggccacgcgggggaagaaaaacagctgagagggcatcaggaaggagtttcgacccgcgctggcgagtcatgagcgccaagtttcccactggcgcgcaaacttgagttacttttgagcgtggatactggcgaagaggctgcgggcggtattagcgtttgcagcgacttggctcgggcagctgacccaagtgtcctgtcttccttcctctgcttgtctctaggctctgaaactgcggagcggccaccggacgccttctggagcaggtagcagcatg

SEQ ID NO: 28 - DNA, 644 bp, Homo sapiens
ccttgagttcagactggaagcctctagaattgtgagaaaatgaatgtctgttgtttaagccacccagtctgtggtatttccttatggcagccccagcaaactaatacaaatagtgtttccacagctgaaacaaaattggaaaatcaccgtcatcctagagagttacaagggctattttaatagaacctgattgttttcctaaattcaccaagcccaggcagaggtcagatgactaattgggataaaagccaactagcttcctcttgctgtttctttagccactggtctgcaggcgttttcttcttctaacttcctctcctgtgacaaaagagataactattagagaaacaaaagtccagaatgctaaggttgccgctttcacttcctctcaccctttagcccagaactgctttgaatacaccaattgctgtggggcggctcgaggaagagaagacaccagtgcctcagaaactgctcggtcagacggtgatagcgagccacgcattcacagggccactgctgctcacagaagcagtgaggatgatgccaggatgatgtctgcctcgcgcctggctgggactctgatcccagccatggccttcctctcctgcgtgagaccagaaagctgggagccctgcgtggag

SEQ ID NO: 29 - DNA, 220 bp, Homo sapiens
taagtgggtaaatattaaattgcccagttgggcaccatcctgaatattatctctaaagaaagaagcaaaaccaggcacagctgatgggttaaccagatatgatacagaaaacatttccttctgctttttggttttaagcctatatttgaagccttagatctctccagcacagtaagcaccaggagtccatgaagaagatggctcctgccatggaatcccc

SEQ ID NO: 30 - DNA, 18 bp, Homo sapiens
tcctccagcccctgctac

SEQ ID NO: 31 - protein, 168 aa, Artificial sequence
  Synthetic construct
GEKPYKCPECGKSFSQRAHLERHQRTHTGEKPYKCPECGKSFSQRAHLERHQRTHTGEKPYKCPECGKSFSTSGELVRHQRTHTGEKPYKCPECGKSFSRSDKLVRHQRTHTGEKPYKCPECGKSFSQSGDLRRHQRTHTGEKPYKCPECGKSFSQSSSLVRHQRTHT

SEQ ID NO: 32 - protein, 168 aa, Artificial sequence
  Synthetic construct
GEKPYKCPECGKSFSTSGHLVRHQRTHTGEKPYKCPECGKSFSQRAHLERHQRTHTGEKPYKCPECGKSFSQSSSLVRHQRTHTGEKPYKCPECGKSFSTSGHLVRHQRTHTGEKPYKCPECGKSFSQSGDLRRHQRTHTGEKPYKCPECGKSFSQSSSLVRHQRTHT

SEQ ID NO: 33 - protein, 168 aa, Artificial sequence
  Synthetic construct
GEKPYKCPECGKSFSTSGHLVRHQRTHTGEKPYKCPECGKSFSRSDKLVRHQRTHTGEKPYKCPECGKSFSDPGALVRHQRTHTGEKPYKCPECGKSFSRSDKLVRHQRTHTGEKPYKCPECGKSFSQSGDLRRHQRTHTGEKPYKCPECGKSFSQSSSLVRHQRTHT

SEQ ID NO: 34 - protein, 168 aa, Artificial sequence
  Synthetic construct
GEKPYKCPECGKSFSQSGDLRRHQRTHTGEKPYKCPECGKSFSQSGDLRRHQRTHTGEKPYKCPECGKSFSQSGDLRRHQRTHTGEKPYKCPECGKSFSTSGHLVRHQRTHTGEKPYKCPECGKSFSRSDKLVRHQRTHTGEKPYKCPECGKSFSTSGSLVRHQRTHT

SEQ ID NO: 35 - protein, 168 aa, Artificial sequence
  Synthetic construct
GEKPYKCPECGKSFSQRAHLERHQRTHTGEKPYKCPECGKSFSTSGHLVRHQRTHTGEKPYKCPECGKSFSQRAHLERHQRTHTGEKPYKCPECGKSFSDPGALVRHQRTHTGEKPYKCPECGKSFSRSDKLVRHQRTHTGEKPYKCPECGKSFSQSSSLVRHQRTHT

SEQ ID NO: 36 - protein, 168 aa, Artificial sequence
  Synthetic construct
GEKPYKCPECGKSFSQRAHLERHQRTHTGEKPYKCPECGKSFSDPGHLVRHQRTHTGEKPYKCPECGKSFSTSGSLVRHQRTHTGEKPYKCPECGKSFSTSGHLVRHQRTHTGEKPYKCPECGKSFSQSGDLRRHQRTHTGEKPYKCPECGKSFSQSSSLVRHQRTHT

SEQ ID NO: 37 - protein, 168 aa, Artificial sequence
  Synthetic construct
GEKPYKCPECGKSFSTSGHLVRHQRTHTGEKPYKCPECGKSFSQRAHLERHQRTHTGEKPYKCPECGKSFSDCRDLARHQRTHTGEKPYKCPECGKSFSRSDDLVRHQRTHTGEKPYKCPECGKSFSQSSSLVRHQRTHTGEKPYKCPECGKSFSQSSSLVRHQRTHT

SEQ ID NO: 38 - DNA, 15 bp, Homo sapiens
ctcctctcccacccc

SEQ ID NO: 39 - protein, 168 aa, Artificial sequence
  Synthetic construct
GEKPYKCPECGKSFSTSGHLVRHQRTHTGEKPYKCPECGKSFSRSDKLVRHQRTHTGEKPYKCPECGKSFSQRAHLERHQRTHTGEKPYKCPECGKSFSRSDELVRHQRTHTGEKPYKCPECGKSFSRSDKLVRHQRTHTGEKPYKCPECGKSFSTSGHLVRHQRTHT

SEQ ID NO: 40 - protein, 168 aa, Artificial sequence
  Synthetic construct
GEKPYKCPECGKSFSRSDNLVRHQRTHTGEKPYKCPECGKSFSQRAHLERHQRTHTGEKPYKCPECGKSFSRSDKLVRHQRTHTGEKPYKCPECGKSFSRSDKLVRHQRTHTGEKPYKCPECGKSFSRSDDLVRHQRTHTGEKPYKCPECGKSFSDCRDLARHQRTHT

SEQ ID NO: 41 - protein, 168 aa, Artificial sequence
  Synthetic construct
GEKPYKCPECGKSFSTSGHLVRHQRTHTGEKPYKCPECGKSFSQSGDLRRHQRTHTGEKPYKCPECGKSFSRSDELVRHQRTHTGEKPYKCPECGKSFSQSGDLRRHQRTHTGEKPYKCPECGKSFSRSDNLVRHQRTHTGEKPYKCPECGKSFSDCRDLARHQRTHT

SEQ ID NO: 42 - protein, 168 aa, Artificial sequence
  Synthetic construct
GEKPYKCPECGKSFSRSDNLVRHQRTHTGEKPYKCPECGKSFSDPGHLVRHQRTHTGEKPYKCPECGKSFSRSDELVRHQRTHTGEKPYKCPECGKSFSQRAHLERHQRTHTGEKPYKCPECGKSFSRSDELVRHQRTHTGEKPYKCPECGKSFSDCRDLARHQRTHT

SEQ ID NO: 43 - protein, 168 aa, Artificial sequence
  Synthetic construct
GEKPYKCPECGKSFSDPGALVRHQRTHTGEKPYKCPECGKSFSQRAHLERHQRTHTGEKPYKCPECGKSFSRSDNLVRHQRTHTGEKPYKCPECGKSFSQRAHLERHQRTHTGEKPYKCPECGKSFSRSDKLVRHQRTHTGEKPYKCPECGKSFSRSDNLVRHQRTHT

SEQ ID NO: 44 - DNA, 15 bp, Homo sapiens
aaggtcggcttcttc

SEQ ID NO: 45 - protein, 168 aa, Artificial sequence
  Synthetic construct
GEKPYKCPECGKSFSTSGNLTEHQRTHTGEKPYKCPECGKSFSTSGNLVRHQRTHTGEKPYKCPECGKSFSRSDELVRHQRTHTGEKPYKCPECGKSFSQSGDLRRHQRTHTGEKPYKCPECGKSFSTSGSLVRHQRTHTGEKPYKCPECGKSFSRSDNLVRHQRTHT

SEQ ID NO: 46 - protein, 168 aa, Artificial sequence
  Synthetic construct
GEKPYKCPECGKSFSTTGALTEHQRTHTGEKPYKCPECGKSFSTSGSLVRHQRTHTGEKPYKCPECGKSFSDCRDLARHQRTHTGEKPYKCPECGKSFSQSSNLVRHQRTHTGEKPYKCPECGKSFSQSSNLVRHQRTHTGEKPYKCPECGKSFSTSGNLVRHQRTHT

SEQ ID NO: 47 - protein, 168 aa, Artificial sequence
  Synthetic construct
GEKPYKCPECGKSFSSRRTCRAHQRTHTGEKPYKCPECGKSFSTSGNLVRHQRTHTGEKPYKCPECGKSFSDCRDLARHQRTHTGEKPYKCPECGKSFSQSSNLVRHQRTHTGEKPYKCPECGKSFSQSSNLVRHQRTHTGEKPYKCPECGKSFSTSGSLVRHQRTHT

SEQ ID NO: 48 - protein, 168 aa, Artificial sequence
  Synthetic construct
GEKPYKCPECGKSFSSKKHLAEHQRTHTGEKPYKCPECGKSFSRSDDLVRHQRTHTGEKPYKCPECGKSFSQSSNLVRHQRTHTGEKPYKCPECGKSFSQSSNLVRHQRTHTGEKPYKCPECGKSFSTSGELVRHQRTHTGEKPYKCPECGKSFSDPGNLVRHQRTHT

SEQ ID NO: 49 - protein, 168 aa, Artificial sequence
  Synthetic construct
GEKPYKCPECGKSFSTTGALTEHQRTHTGEKPYKCPECGKSFSDCRDLARHQRTHTGEKPYKCPECGKSFSDCRDLARHQRTHTGEKPYKCPECGKSFSQSSNLVRHQRTHTGEKPYKCPECGKSFSQSSNLVRHQRTHTGEKPYKCPECGKSFSTSGSLVRHQRTHT

SEQ ID NO: 50 - protein, 168 aa, Artificial sequence
  Synthetic construct
GEKPYKCPECGKSFSRSDKLTEHQRTHTGEKPYKCPECGKSFSRSDNLVRHQRTHTGEKPYKCPECGKSFSTSGSLVRHQRTHTGEKPYKCPECGKSFSDPGNLVRHQRTHTGEKPYKCPECGKSFSQSSSLVRHQRTHTGEKPYKCPECGKSFSQSSSLVRHQRTHT

SEQ ID NO: 51 - DNA, 15 bp, Homo sapiens
cggagccacgcgctg

SEQ ID NO: 52 - protein, 168 aa, Artificial sequence
  Synthetic construct
GEKPYKCPECGKSFSTSGSLVRHQRTHTGEKPYKCPECGKSFSRNDALTEHQRTHTGEKPYKCPECGKSFSRSDDLVRHQRTHTGEKPYKCPECGKSFSTSGNLVRHQRTHTGEKPYKCPECGKSFSQSGHLTEHQRTHTGEKPYKCPECGKSFSRSDKLTEHQRTHT

SEQ ID NO: 53 - DNA, 18 bp, Homo sapiens
cggctcctcaacggcctc

SEQ ID NO: 54 - protein, 168 aa, Artificial sequence
  Synthetic construct
GEKPYKCPECGKSFSTKNSLTEHQRTHTGEKPYKCPECGKSFSQSSSLVRHQRTHTGEKPYKCPECGKSFSRSDNLVRHQRTHTGEKPYKCPECGKSFSTSGSLVRHQRTHTGEKPYKCPECGKSFSDPGNLVRHQRTHTGEKPYKCPECGKSFSQSSSLVRHQRTHT

SEQ ID NO: 55 - protein, 168 aa, Artificial sequence
  Synthetic construct
GEKPYKCPECGKSFSRSDKLTEHQRTHTGEKPYKCPECGKSFSQSSSLVRHQRTHTGEKPYKCPECGKSFSQRAHLERHQRTHTGEKPYKCPECGKSFSRSDNLVRHQRTHTGEKPYKCPECGKSFSQSSSLVRHQRTHTGEKPYKCPECGKSFSDPGNLVRHQRTHT

SEQ ID NO: 56 - protein, 168 aa, Artificial sequence
  Synthetic construct
GEKPYKCPECGKSFSQNSTLTEHQRTHTGEKPYKCPECGKSFSQSSSLVRHQRTHTGEKPYKCPECGKSFSQRAHLERHQRTHTGEKPYKCPECGKSFSRSDNLVRHQRTHTGEKPYKCPECGKSFSQSSNLVRHQRTHTGEKPYKCPECGKSFSDCRDLARHQRTHT

SEQ ID NO: 57 - protein, 168 aa, Artificial sequence
  Synthetic construct
GEKPYKCPECGKSFSTSHSLTEHQRTHTGEKPYKCPECGKSFSQSSSLVRHQRTHTGEKPYKCPECGKSFSQRAHLERHQRTHTGEKPYKCPECGKSFSRSDNLVRHQRTHTGEKPYKCPECGKSFSQSSNLVRHQRTHTGEKPYKCPECGKSFSDCRDLARHQRTHT

SEQ ID NO: 58 - DNA, 18 bp, Homo sapiens
ccacccgtgggccctggc

SEQ ID NO: 59 - protein, 168 aa, Artificial sequence
  Synthetic construct
GEKPYKCPECGKSFSQRAHLERHQRTHTGEKPYKCPECGKSFSQSGHLTEHQRTHTGEKPYKCPECGKSFSRSDNLVRHQRTHTGEKPYKCPECGKSFSDPGNLVRHQRTHTGEKPYKCPECGKSFSRSDKLTEHQRTHTGEKPYKCPECGKSFSTTGALTEHQRTHT

SEQ ID NO: 60 - protein, 168 aa, Artificial sequence
  Synthetic construct
GEKPYKCPECGKSFSRSDELVRHQRTHTGEKPYKCPECGKSFSRSDKLTEHQRTHTGEKPYKCPECGKSFSQSSSLVRHQRTHTGEKPYKCPECGKSFSDPGHLVRHQRTHTGEKPYKCPECGKSFSRADNLTEHQRTHTGEKPYKCPECGKSFSTKNSLTEHQRTHT

SEQ ID NO: 61 - protein, 168 aa, Artificial sequence
  Synthetic construct
GEKPYKCPECGKSFSQRAHLERHQRTHTGEKPYKCPECGKSFSRNDTLTEHQRTHTGEKPYKCPECGKSFSQRAHLERHQRTHTGEKPYKCPECGKSFSQSGDLRRHQRTHTGEKPYKCPECGKSFSTTGALTEHQRTHTGEKPYKCPECGKSFSRSDKLTEHQRTHT

SEQ ID NO: 62 - protein, 168 aa, Artificial sequence
  Synthetic construct
GEKPYKCPECGKSFSRSDELVRHQRTHTGEKPYKCPECGKSFSRSDKLTEHQRTHTGEKPYKCPECGKSFSQSSSLVRHQRTHTGEKPYKCPECGKSFSDPGHLVRHQRTHTGEKPYKCPECGKSFSRNDTLTEHQRTHTGEKPYKCPECGKSFSSRRTCRAHQRTHT

SEQ ID NO: 63 - protein, 168 aa, Artificial sequence
  Synthetic construct
GEKPYKCPECGKSFSTSGNLVRHQRTHTGEKPYKCPECGKSFSQSGHLTEHQRTHTGEKPYKCPECGKSFSTSGNLVRHQRTHTGEKPYKCPECGKSFSTSGNLVRHQRTHTGEKPYKCPECGKSFSRSDKLTEHQRTHTGEKPYKCPECGKSFSSRRTCRAHQRTHT

SEQ ID NO: 64 - protein, 168 aa, Artificial sequence
  Synthetic construct
GEKPYKCPECGKSFSTSGNLVRHQRTHTGEKPYKCPECGKSFSQSGHLTEHQRTHTGEKPYKCPECGKSFSDPGHLVRHQRTHTGEKPYKCPECGKSFSQSSSLVRHQRTHTGEKPYKCPECGKSFSRSDKLTEHQRTHTGEKPYKCPECGKSFSSRRTCRAHQRTHT

SEQ ID NO: 65 - DNA, 18 bp, Homo sapiens
ctcctccacatcccccac

SEQ ID NO: 66 - protein, 168 aa, Artificial sequence
  Synthetic construct
GEKPYKCPECGKSFSTSGHLVRHQRTHTGEKPYKCPECGKSFSDPGALVRHQRTHTGEKPYKCPECGKSFSTSGELVRHQRTHTGEKPYKCPECGKSFSTSGNLVRHQRTHTGEKPYKCPECGKSFSRSDKLVRHQRTHTGEKPYKCPECGKSFSTSGSLVRHQRTHT

SEQ ID NO: 67 - protein, 168 aa, Artificial sequence
  Synthetic construct
GEKPYKCPECGKSFSDPGHLVRHQRTHTGEKPYKCPECGKSFSRSDELVRHQRTHTGEKPYKCPECGKSFSTSGNLVRHQRTHTGEKPYKCPECGKSFSRSDKLVRHQRTHTGEKPYKCPECGKSFSQSGDLRRHQRTHTGEKPYKCPECGKSFSDPGHLVRHQRTHT

SEQ ID NO: 68 - protein, 168 aa, Artificial sequence
  Synthetic construct
GEKPYKCPECGKSFSDCRDLARHQRTHTGEKPYKCPECGKSFSTSGHLVRHQRTHTGEKPYKCPECGKSFSRSDELVRHQRTHTGEKPYKCPECGKSFSTSGNLVRHQRTHTGEKPYKCPECGKSFSRSDKLVRHQRTHTGEKPYKCPECGKSFSDPGHLVRHQRTHT

SEQ ID NO: 69 - protein, 168 aa, Artificial sequence
  Synthetic construct
GEKPYKCPECGKSFSRSDDLVRHQRTHTGEKPYKCPECGKSFSRSDELVRHQRTHTGEKPYKCPECGKSFSTSGNLVRHQRTHTGEKPYKCPECGKSFSRSDKLVRHQRTHTGEKPYKCPECGKSFSTSGSLVRHQRTHTGEKPYKCPECGKSFSDPGALVRHQRTHT

SEQ ID NO: 70 - protein, 168 aa, Artificial sequence
  Synthetic construct
GEKPYKCPECGKSFSTSGHLVRHQRTHTGEKPYKCPECGKSFSDPGHLVRHQRTHTGEKPYKCPECGKSFSRSDELVRHQRTHTGEKPYKCPECGKSFSTSGNLVRHQRTHTGEKPYKCPECGKSFSRSDKLVRHQRTHTGEKPYKCPECGKSFSDCRDLARHQRTHT

SEQ ID NO: 71 - protein, 168 aa, Artificial sequence
  Synthetic construct
GEKPYKCPECGKSFSQRAHLERHQRTHTGEKPYKCPECGKSFSDCRDLARHQRTHTGEKPYKCPECGKSFSRSDELVRHQRTHTGEKPYKCPECGKSFSTSGNLVRHQRTHTGEKPYKCPECGKSFSRSDKLVRHQRTHTGEKPYKCPECGKSFSDPGNLVRHQRTHT

SEQ ID NO: 72 - protein, 168 aa, Artificial sequence
  Synthetic construct
GEKPYKCPECGKSFSTSGELVRHQRTHTGEKPYKCPECGKSFSRSDDLVRHQRTHTGEKPYKCPECGKSFSTSGNLVRHQRTHTGEKPYKCPECGKSFSRSDKLVRHQRTHTGEKPYKCPECGKSFSRSDKLVRHQRTHTGEKPYKCPECGKSFSDPGHLVRHQRTHT

SEQ ID NO: 73 - protein, 168 aa, Artificial sequence
  Synthetic construct
GEKPYKCPECGKSFSQSSNLVRHQRTHTGEKPYKCPECGKSFSRSDELVRHQRTHTGEKPYKCPECGKSFSRSDDLVRHQRTHTGEKPYKCPECGKSFSTSGELVRHQRTHTGEKPYKCPECGKSFSRSDKLVRHQRTHTGEKPYKCPECGKSFSRSDDLVRHQRTHT

SEQ ID NO: 74 - protein, 168 aa, Artificial sequence
  Synthetic construct
GEKPYKCPECGKSFSDPGHLVRHQRTHTGEKPYKCPECGKSFSDCRDLARHQRTHTGEKPYKCPECGKSFSRSDELVRHQRTHTGEKPYKCPECGKSFSTSGNLVRHQRTHTGEKPYKCPECGKSFSRSDKLVRHQRTHTGEKPYKCPECGKSFSTSGSLVRHQRTHT

SEQ ID NO: 75 - protein, 168 aa, Artificial sequence
  Synthetic construct
GEKPYKCPECGKSFSDPGNLVRHQRTHTGEKPYKCPECGKSFSRSDDLVRHQRTHTGEKPYKCPECGKSFSTSGNLVRHQRTHTGEKPYKCPECGKSFSRSDKLVRHQRTHTGEKPYKCPECGKSFSDPGALVRHQRTHTGEKPYKCPECGKSFSDCRDLARHQRTHT

SEQ ID NO: 76 - protein, 168 aa, Artificial sequence
  Synthetic construct
GEKPYKCPECGKSFSDPGHLVRHQRTHTGEKPYKCPECGKSFSRSDDLVRHQRTHTGEKPYKCPECGKSFSTSGNLVRHQRTHTGEKPYKCPECGKSFSRSDKLVRHQRTHTGEKPYKCPECGKSFSRSDKLVRHQRTHTGEKPYKCPECGKSFSDPGALVRHQRTHT

SEQ ID NO: 77 - protein, 168 aa, Artificial sequence
  Synthetic construct
GEKPYKCPECGKSFSRSDDLVRHQRTHTGEKPYKCPECGKSFSRSDELVRHQRTHTGEKPYKCPECGKSFSTSGNLVRHQRTHTGEKPYKCPECGKSFSRSDKLVRHQRTHTGEKPYKCPECGKSFSTSGSLVRHQRTHTGEKPYKCPECGKSFSDCRDLARHQRTHT

SEQ ID NO: 78 - protein, 168 aa, Artificial sequence
  Synthetic construct
GEKPYKCPECGKSFSRSDDLVRHQRTHTGEKPYKCPECGKSFSRSDELVRHQRTHTGEKPYKCPECGKSFSTSGNLVRHQRTHTGEKPYKCPECGKSFSRSDKLVRHQRTHTGEKPYKCPECGKSFSQSSSLVRHQRTHTGEKPYKCPECGKSFSDCRDLARHQRTHT

SEQ ID NO: 79 - protein, 168 aa, Artificial sequence
  Synthetic construct
GEKPYKCPECGKSFSQRAHLERHQRTHTGEKPYKCPECGKSFSDPGHLVRHQRTHTGEKPYKCPECGKSFSRSDDLVRHQRTHTGEKPYKCPECGKSFSTSGNLVRHQRTHTGEKPYKCPECGKSFSRSDKLVRHQRTHTGEKPYKCPECGKSFSDPGALVRHQRTHT

SEQ ID NO: 80 - protein, 140 aa, Artificial sequence
  Synthetic construct
GEKPYKCPECGKSFSDPGNLVRHQRTHTGEKPYKCPECGKSFSQSSSLVRHQRTHTGEKPYKCPECGKSFSTSGNLVRHQRTHTGEKPYKCPECGKSFSRSDKLVRHQRTHTGEKPYKCPECGKSFSDPGALVRHQRTHT

SEQ ID NO: 81 - DNA, 18 bp, Homo sapiens
ggcctggaagggggcggc

SEQ ID NO: 82 - protein, 168 aa, Artificial sequence
  Synthetic construct
GEKPYKCPECGKSFSTSGELVRHQRTHTGEKPYKCPECGKSFSQSSSLVRHQRTHTGEKPYKCPECGKSFSQSSSLVRHQRTHTGEKPYKCPECGKSFSRSDELVRHQRTHTGEKPYKCPECGKSFSRSDKLTEHQRTHTGEKPYKCPECGKSFSDPGHLVRHQRTHT

SEQ ID NO: 83 - protein, 168 aa, Artificial sequence
  Synthetic construct
GEKPYKCPECGKSFSQSGDLRRHQRTHTGEKPYKCPECGKSFSQSGDLRRHQRTHTGEKPYKCPECGKSFSRSDELVRHQRTHTGEKPYKCPECGKSFSTSGNLVRHQRTHTGEKPYKCPECGKSFSRSDKLTEHQRTHTGEKPYKCPECGKSFSRSDELVRHQRTHT

SEQ ID NO: 84 - protein, 168 aa, Artificial sequence
  Synthetic construct
GEKPYKCPECGKSFSDPGALVRHQRTHTGEKPYKCPECGKSFSTSGNLVRHQRTHTGEKPYKCPECGKSFSQSSSLVRHQRTHTGEKPYKCPECGKSFSDCRDLARHQRTHTGEKPYKCPECGKSFSRSDKLTEHQRTHTGEKPYKCPECGKSFSRSDNLVRHQRTHT

SEQ ID NO: 85 - protein, 168 aa, Artificial sequence
  Synthetic construct
GEKPYKCPECGKSFSQSSSLVRHQRTHTGEKPYKCPECGKSFSQSSSLVRHQRTHTGEKPYKCPECGKSFSDCRDLARHQRTHTGEKPYKCPECGKSFSRSDKLVRHQRTHTGEKPYKCPECGKSFSRADNLTEHQRTHTGEKPYKCPECGKSFSQSSSLVRHQRTHT

SEQ ID NO: 86 - protein, 168 aa, Artificial sequence
  Synthetic construct
GEKPYKCPECGKSFSQSGDLRRHQRTHTGEKPYKCPECGKSFSQSGDLRRHQRTHTGEKPYKCPECGKSFSTSGNLVRHQRTHTGEKPYKCPECGKSFSRSDKLVRHQRTHTGEKPYKCPECGKSFSRNDALTEHQRTHTGEKPYKCPECGKSFSDPGHLVRHQRTHT

SEQ ID NO: 87 - protein, 168 aa, Artificial sequence
  Synthetic construct
GEKPYKCPECGKSFSRSDKLVRHQRTHTGEKPYKCPECGKSFSQSSNLVRHQRTHTGEKPYKCPECGKSFSRSDELVRHQRTHTGEKPYKCPECGKSFSDPGHLVRHQRTHTGEKPYKCPECGKSFSTTGALTEHQRTHTGEKPYKCPECGKSFSDCRDLARHQRTHT

SEQ ID NO: 88 - protein, 168 aa, Artificial sequence
  Synthetic construct
GEKPYKCPECGKSFSQSGDLRRHQRTHTGEKPYKCPECGKSFSQSGDLRRHQRTHTGEKPYKCPECGKSFSQSGDLRRHQRTHTGEKPYKCPECGKSFSRSDKLVRHQRTHTGEKPYKCPECGKSFSSKKHLAEHQRTHTGEKPYKCPECGKSFSDPGHLVRHQRTHT

SEQ ID NO: 89 - protein, 168 aa, Artificial sequence
  Synthetic construct
GEKPYKCPECGKSFSRSDELVRHQRTHTGEKPYKCPECGKSFSTSGSLVRHQRTHTGEKPYKCPECGKSFSQSSSLVRHQRTHTGEKPYKCPECGKSFSDCRDLARHQRTHTGEKPYKCPECGKSFSRSDKLTEHQRTHTGEKPYKCPECGKSFSRSDNLVRHQRTHT

SEQ ID NO: 90 - protein, 168 aa, Artificial sequence
  Synthetic construct
GEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSTSGNLVRHQRTHTGEKPYKCPECGKSFSRNDALTEHQRTHTGEKPYKCPECGKSFSDCRDLARHQRTHT

SEQ ID NO: 91 - protein, 168 aa, Artificial sequence
  Synthetic construct
GEKPYKCPECGKSFSDCRDLARHQRTHTGEKPYKCPECGKSFSTSGNLVRHQRTHTGEKPYKCPECGKSFSQSSSLVRHQRTHTGEKPYKCPECGKSFSDCRDLARHQRTHTGEKPYKCPECGKSFSRSDKLTEHQRTHTGEKPYKCPECGKSFSRSDNLVRHQRTHT

SEQ ID NO: 92 - protein, 168 aa, Artificial sequence
  Synthetic construct
GEKPYKCPECGKSFSTSGHLVRHQRTHTGEKPYKCPECGKSFSDPGHLVRHQRTHTGEKPYKCPECGKSFSRSDELVRHQRTHTGEKPYKCPECGKSFSTSGNLVRHQRTHTGEKPYKCPECGKSFSRSDKLTEHQRTHTGEKPYKCPECGKSFSTSGHLVRHQRTHT

SEQ ID NO: 93 - protein, 168 aa, Artificial sequence
  Synthetic construct
GEKPYKCPECGKSFSTSGHLVRHQRTHTGEKPYKCPECGKSFSTSGELVRHQRTHTGEKPYKCPECGKSFSQSGDLRRHQRTHTGEKPYKCPECGKSFSRSDDLVRHQRTHTGEKPYKCPECGKSFSRSDKLTEHQRTHTGEKPYKCPECGKSFSDPGALVRHQRTHT

SEQ ID NO: 94 - protein, 168 aa, Artificial sequence
  Synthetic construct
GEKPYKCPECGKSFSDPGALVRHQRTHTGEKPYKCPECGKSFSQSGDLRRHQRTHTGEKPYKCPECGKSFSRSDDLVRHQRTHTGEKPYKCPECGKSFSRSDKLVRHQRTHTGEKPYKCPECGKSFSRNDALTEHQRTHTGEKPYKCPECGKSFSDCRDLARHQRTHT

SEQ ID NO: 95 - protein, 168 aa, Artificial sequence
  Synthetic construct
GEKPYKCPECGKSFSQSGDLRRHQRTHTGEKPYKCPECGKSFSQSGDLRRHQRTHTGEKPYKCPECGKSFSRSDELVRHQRTHTGEKPYKCPECGKSFSTSGNLVRHQRTHTGEKPYKCPECGKSFSRSDKLTEHQRTHTGEKPYKCPECGKSFSQRAHLERHQRTHT

SEQ ID NO: 96 - DNA, 18 bp, Homo sapiens
ggaggagacggggaggac

SEQ ID NO: 97 - protein, 168 aa, Artificial sequence
  Synthetic construct
GEKPYKCPECGKSFSDPGNLVRHQRTHTGEKPYKCPECGKSFSRSDNLVRHQRTHTGEKPYKCPECGKSFSRSDKLVRHQRTHTGEKPYKCPECGKSFSDPGNLVRHQRTHTGEKPYKCPECGKSFSQRAHLERHQRTHTGEKPYKCPECGKSFSQRAHLERHQRTHT

SEQ ID NO: 98 - protein, 168 aa, Artificial sequence
  Synthetic construct
GEKPYKCPECGKSFSQSSNLVRHQRTHTGEKPYKCPECGKSFSRSDELVRHQRTHTGEKPYKCPECGKSFSDPGHLVRHQRTHTGEKPYKCPECGKSFSDPGNLVRHQRTHTGEKPYKCPECGKSFSQRAHLERHQRTHTGEKPYKCPECGKSFSDPGHLVRHQRTHT

SEQ ID NO: 99 - protein, 168 aa, Artificial sequence
  Synthetic construct
GEKPYKCPECGKSFSRSDNLVRHQRTHTGEKPYKCPECGKSFSRSDKLVRHQRTHTGEKPYKCPECGKSFSDPGALVRHQRTHTGEKPYKCPECGKSFSDPGNLVRHQRTHTGEKPYKCPECGKSFSRSDKLVRHQRTHTGEKPYKCPECGKSFSDPGHLVRHQRTHT

SEQ ID NO: 100 - protein, 168 aa, Artificial sequence
  Synthetic construct
GEKPYKCPECGKSFSRSDNLVRHQRTHTGEKPYKCPECGKSFSRSDNLVRHQRTHTGEKPYKCPECGKSFSQSSSLVRHQRTHTGEKPYKCPECGKSFSQSGDLRRHQRTHTGEKPYKCPECGKSFSRSDELVRHQRTHTGEKPYKCPECGKSFSRSDNLVRHQRTHT

SEQ ID NO: 101 - protein, 168 aa, Artificial sequence
  Synthetic construct
GEKPYKCPECGKSFSRSDELVRHQRTHTGEKPYKCPECGKSFSRSDKLVRHQRTHTGEKPYKCPECGKSFSRSDKLVRHQRTHTGEKPYKCPECGKSFSDPGNLVRHQRTHTGEKPYKCPECGKSFSQRAHLERHQRTHTGEKPYKCPECGKSFSTSGNLVRHQRTHT

SEQ ID NO: 102 - protein, 164 aa, Artificial sequence
  Synthetic construct
GEKPYKCPECGKSFSIVRHQRTHTGEKPYKCPECGKSFSRSDNLVRHQRTHTGEKPYKCPECGKSFSDPGHLVRHQRTHTGEKPYKCPECGKSFSDPGNLVRHQRTHTGEKPYKCPECGKSFSQSSSLVRHQRTHTGEKPYKCPECGKSFSDPGHLVRHQRTHT

SEQ ID NO: 103 - protein, 168 aa, Artificial sequence
  Synthetic construct
GEKPYKCPECGKSFSRSDNLVRHQRTHTGEKPYKCPECGKSFSTSGHLVRHQRTHTGEKPYKCPECGKSFSDPGNLVRHQRTHTGEKPYKCPECGKSFSQSGDLRRHQRTHTGEKPYKCPECGKSFSDPGHLVRHQRTHTGEKPYKCPECGKSFSDCRDLARHQRTHT

SEQ ID NO: 104 - protein, 168 aa, Artificial sequence
  Synthetic construct
GEKPYKCPECGKSFSRSDNLVRHQRTHTGEKPYKCPECGKSFSDPGHLVRHQRTHTGEKPYKCPECGKSFSDPGNLVRHQRTHTGEKPYKCPECGKSFSTSGHLVRHQRTHTGEKPYKCPECGKSFSQRAHLERHQRTHTGEKPYKCPECGKSFSDCRDLARHQRTHT

SEQ ID NO: 105 - protein, 168 aa, Artificial sequence
  Synthetic construct
GEKPYKCPECGKSFSRSDNLVRHQRTHTGEKPYKCPECGKSFSDPGHLVRHQRTHTGEKPYKCPECGKSFSDPGALVRHQRTHTGEKPYKCPECGKSFSQRAHLERHQRTHTGEKPYKCPECGKSFSQRAHLERHQRTHTGEKPYKCPECGKSFSRSDDLVRHQRTHT

SEQ ID NO: 106 - protein, 168 aa, Artificial sequence
  Synthetic construct
GEKPYKCPECGKSFSRSDNLVRHQRTHTGEKPYKCPECGKSFSRSDELVRHQRTHTGEKPYKCPECGKSFSDCRDLARHQRTHTGEKPYKCPECGKSFSQRAHLERHQRTHTGEKPYKCPECGKSFSQRAHLERHQRTHTGEKPYKCPECGKSFSDPGHLVRHQRTHT

SEQ ID NO: 107 - protein, 168 aa, Artificial sequence
  Synthetic construct
GEKPYKCPECGKSFSDPGALVRHQRTHTGEKPYKCPECGKSFSRSDNLVRHQRTHTGEKPYKCPECGKSFSDCRDLARHQRTHTGEKPYKCPECGKSFSDPGNLVRHQRTHTGEKPYKCPECGKSFSQRAHLERHQRTHTGEKPYKCPECGKSFSDPGHLVRHQRTHT

SEQ ID NO: 108 - protein, 168 aa, Artificial sequence
  Synthetic construct
GEKPYKCPECGKSFSTSGSLVRHQRTHTGEKPYKCPECGKSFSDPGNLVRHQRTHTGEKPYKCPECGKSFSDPGHLVRHQRTHTGEKPYKCPECGKSFSRSDNLVRHQRTHTGEKPYKCPECGKSFSRSDKLVRHQRTHTGEKPYKCPECGKSFSDCRDLARHQRTHT

SEQ ID NO: 109 - protein, 168 aa, Artificial sequence
  Synthetic construct
GEKPYKCPECGKSFSDPGALVRHQRTHTGEKPYKCPECGKSFSRSDNLVRHQRTHTGEKPYKCPECGKSFSRSDELVRHQRTHTGEKPYKCPECGKSFSTSGELVRHQRTHTGEKPYKCPECGKSFSDPGNLVRHQRTHTGEKPYKCPECGKSFSRSDELVRHQRTHT

SEQ ID NO: 110 - protein, 168 aa, Artificial sequence
  Synthetic construct
GEKPYKCPECGKSFSTSGHLVRHQRTHTGEKPYKCPECGKSFSRSDNLVRHQRTHTGEKPYKCPECGKSFSDPGALVRHQRTHTGEKPYKCPECGKSFSDPGHLVRHQRTHTGEKPYKCPECGKSFSQRAHLERHQRTHTGEKPYKCPECGKSFSQRAHLERHQRTHT

SEQ ID NO: 111 - protein, 168 aa, Artificial sequence
  Synthetic construct
GEKPYKCPECGKSFSRSDNLVRHQRTHTGEKPYKCPECGKSFSRSDKLVRHQRTHTGEKPYKCPECGKSFSDPGHLVRHQRTHTGEKPYKCPECGKSFSDPGNLVRHQRTHTGEKPYKCPECGKSFSTSGNLVRHQRTHTGEKPYKCPECGKSFSTSGSLVRHQRTHT

SEQ ID NO: 112 - protein, 168 aa, Artificial sequence
  Synthetic construct
GEKPYKCPECGKSFSRSDKLVRHQRTHTGEKPYKCPECGKSFSDPGNLVRHQRTHTGEKPYKCPECGKSFSRSDELVRHQRTHTGEKPYKCPECGKSFSRSDELVRHQRTHTGEKPYKCPECGKSFSQSSSLVRHQRTHTGEKPYKCPECGKSFSQRAHLERHQRTHT

SEQ ID NO: 113 - protein, 168 aa, Artificial Sequence
  Synthetic construct
GEKPYKCPECGKSFSDPGNLVRHQRTHTGEKPYKCPECGKSFSRSDELVRHQRTHTGEKPYKCPECGKSFSRSDNLVRHQRTHTGEKPYKCPECGKSFSDPGNLVRHQRTHTGEKPYKCPECGKSFSTSGSLVRHQRTHTGEKPYKCPECGKSFSDPGHLVRHQRTHT

SEQ ID NO: 114 - protein, 168 aa, Artificial Sequence
  Synthetic construct
GEKPYKCPECGKSFSRSDNLVRHQRTHTGEKPYKCPECGKSFSRSDDLVRHQRTHTGEKPYKCPECGKSFSDPGNLVRHQRTHTGEKPYKCPECGKSFSQRAHLERHQRTHTGEKPYKCPECGKSFSDPGHLVRHQRTHTGEKPYKCPECGKSFSDPGHLVRHQRTHT

SEQ ID NO: 115 - protein, 168 aa, Artificial Sequence
  Synthetic construct
GEKPYKCPECGKSFSDPGNLVRHQRTHTGEKPYKCPECGKSFSRSDELVRHQRTHTGEKPYKCPECGKSFSTSGHLVRHQRTHTGEKPYKCPECGKSFSDPGNLVRHQRTHTGEKPYKCPECGKSFSRSDELVRHQRTHTGEKPYKCPECGKSFSTSGSLVRHQRTHT

SEQ ID NO: 116 - protein, 168 aa, Artificial Sequence
  Synthetic construct
GEKPYKCPECGKSFSQSSNLVRHQRTHTGEKPYKCPECGKSFSTSGNLVRHQRTHTGEKPYKCPECGKSFSRSDELVRHQRTHTGEKPYKCPECGKSFSTSGHLVRHQRTHTGEKPYKCPECGKSFSDPGHLVRHQRTHTGEKPYKCPECGKSFSRSDELVRHQRTHT

SEQ ID NO: 117 - protein, 168 aa, Artificial Sequence
  Synthetic construct
GEKPYKCPECGKSFSRSDNLVRHQRTHTGEKPYKCPECGKSFSRSDKLVRHQRTHTGEKPYKCPECGKSFSDCRDLARHQRTHTGEKPYKCPECGKSFSQRAHLERHQRTHTGEKPYKCPECGKSFSDPGHLVRHQRTHTGEKPYKCPECGKSFSDPGALVRHQRTHT

SEQ ID NO: 118 - protein, 168 aa, Artificial Sequence
  Synthetic construct
GEKPYKCPECGKSFSRSDNLVRHQRTHTGEKPYKCPECGKSFSRSDELVRHQRTHTGEKPYKCPECGKSFSTSGHLVRHQRTHTGEKPYKCPECGKSFSDPGNLVRHQRTHTGEKPYKCPECGKSFSQSSSLVRHQRTHTGEKPYKCPECGKSFSTSGELVRHQRTHT

SEQ ID NO: 119 - DNA, 18 bp, Homo sapiens
ctcgggcaactactactg

SEQ ID NO: 120 - protein, 168 aa, Artificial Sequence
  Synthetic construct
GEKPYKCPECGKSFSRNDALTEHQRTHTGEKPYKCPECGKSFSSRRTCRAHQRTHTGEKPYKCPECGKSFSQNSTLTEHQRTHTGEKPYKCPECGKSFSSKKHLAEHQRTHTGEKPYKCPECGKSFSRSDNLVRHQRTHTGEKPYKCPECGKSFSRSDKLTEHQRTHT

SEQ ID NO: 121 - protein, 168 aa, Artificial Sequence
  Synthetic construct
GEKPYKCPECGKSFSRNDALTEHQRTHTGEKPYKCPECGKSFSTKNSLTEHQRTHTGEKPYKCPECGKSFSRSDKLTEHQRTHTGEKPYKCPECGKSFSRSDKLTEHQRTHTGEKPYKCPECGKSFSRSDNLVRHQRTHTGEKPYKCPECGKSFSRSDKLTEHQRTHT

SEQ ID NO: 122 - protein, 168 aa, Artificial Sequence
  Synthetic construct
GEKPYKCPECGKSFSSRRTCRAHQRTHTGEKPYKCPECGKSFSRNDALTEHQRTHTGEKPYKCPECGKSFSRSDKLTEHQRTHTGEKPYKCPECGKSFSRSDKLTEHQRTHTGEKPYKCPECGKSFSRSDNLVRHQRTHTGEKPYKCPECGKSFSRSDKLTEHQRTHT

SEQ ID NO: 123 - protein, 168 aa, Artificial Sequence
  Synthetic construct
GEKPYKCPECGKSFSSKKHLAEHQRTHTGEKPYKCPECGKSFSTSGNLTEHQRTHTGEKPYKCPECGKSFSRSDKLTEHQRTHTGEKPYKCPECGKSFSRSDKLTEHQRTHTGEKPYKCPECGKSFSRSDNLVRHQRTHTGEKPYKCPECGKSFSRSDKLTEHQRTHT

SEQ ID NO: 124 - protein, 168 aa, Artificial Sequence
  Synthetic construct
GEKPYKCPECGKSFSRSDKLTEHQRTHTGEKPYKCPECGKSFSSRRTCRAHQRTHTGEKPYKCPECGKSFSTKNSLTEHQRTHTGEKPYKCPECGKSFSTKNSLTEHQRTHTGEKPYKCPECGKSFSDCRDLARHQRTHTGEKPYKCPECGKSFSSKKHLAEHQRTHT

SEQ ID NO: 125 - protein, 168 aa, Artificial Sequence
  Synthetic construct
GEKPYKCPECGKSFSRSDKLTEHQRTHTGEKPYKCPECGKSFSTKNSLTEHQRTHTGEKPYKCPECGKSFSSRRTCRAHQRTHTGEKPYKCPECGKSFSRSDKLTEHQRTHTGEKPYKCPECGKSFSRSDNLVRHQRTHTGEKPYKCPECGKSFSRSDKLTEHQRTHT

SEQ ID NO: 126 - protein, 168 aa, Artificial Sequence
  Synthetic construct
GEKPYKCPECGKSFSTTGALTEHQRTHTGEKPYKCPECGKSFSRSDKLTEHQRTHTGEKPYKCPECGKSFSTKNSLTEHQRTHTGEKPYKCPECGKSFSSKKHLAEHQRTHTGEKPYKCPECGKSFSRSDNLVRHQRTHTGEKPYKCPECGKSFSRSDKLTEHQRTHT

SEQ ID NO: 127 - protein, 168 aa, Artificial Sequence
  Synthetic construct
GEKPYKCPECGKSFSRNDALTEHQRTHTGEKPYKCPECGKSFSTTGALTEHQRTHTGEKPYKCPECGKSFSTSGNLTEHQRTHTGEKPYKCPECGKSFSTTGALTEHQRTHTGEKPYKCPECGKSFSTSGNLVRHQRTHTGEKPYKCPECGKSFSRSDKLTEHQRTHT

SEQ ID NO: 128 - protein, 168 aa, Artificial Sequence
  Synthetic construct
GEKPYKCPECGKSFSTKNSLTEHQRTHTGEKPYKCPECGKSFSSRRTCRAHQRTHTGEKPYKCPECGKSFSRSDKLTEHQRTHTGEKPYKCPECGKSFSRSDKLTEHQRTHTGEKPYKCPECGKSFSRSDNLVRHQRTHTGEKPYKCPECGKSFSRSDKLTEHQRTHT

SEQ ID NO: 129 - protein, 168 aa, Artificial Sequence
  Synthetic construct
GEKPYKCPECGKSFSQSGNLTEHQRTHTGEKPYKCPECGKSFSHTGHLLEHQRTHTGEKPYKCPECGKSFSRSDKLTEHQRTHTGEKPYKCPECGKSFSRSDKLTEHQRTHTGEKPYKCPECGKSFSRSDNLVRHQRTHTGEKPYKCPECGKSFSRSDKLTEHQRTHT

SEQ ID NO: 130 - protein, 168 aa, Artificial Sequence
  Synthetic construct
GEKPYKCPECGKSFSRNDALTEHQRTHTGEKPYKCPECGKSFSTSGNLTEHQRTHTGEKPYKCPECGKSFSRSDKLTEHQRTHTGEKPYKCPECGKSFSRSDKLTEHQRTHTGEKPYKCPECGKSFSRSDNLVRHQRTHTGEKPYKCPECGKSFSRSDKLTEHQRTHT

SEQ ID NO: 131 - protein, 168 aa, Artificial Sequence
  Synthetic construct
GEKPYKCPECGKSFSTTGALTEHQRTHTGEKPYKCPECGKSFSSRRTCRAHQRTHTGEKPYKCPECGKSFSRSDKLTEHQRTHTGEKPYKCPECGKSFSRSDKLTEHQRTHTGEKPYKCPECGKSFSRSDNLVRHQRTHTGEKPYKCPECGKSFSRSDKLTEHQRTHT

SEQ ID NO: 132 - protein, 168 aa, Artificial Sequence; identical to SEQ ID NO: 128
  Synthetic construct

SEQ ID NO: 133 - protein, 168 aa, Artificial Sequence
  Synthetic construct
GEKPYKCPECGKSFSTTGALTEHQRTHTGEKPYKCPECGKSFSTKNSLTEHQRTHTGEKPYKCPECGKSFSRSDKLTEHQRTHTGEKPYKCPECGKSFSRSDKLTEHQRTHTGEKPYKCPECGKSFSRSDNLVRHQRTHTGEKPYKCPECGKSFSRSDKLTEHQRTHT

SEQ ID NO: 134 - protein, 168 aa, Artificial Sequence
  Synthetic construct
GEKPYKCPECGKSFSSKKHLAEHQRTHTGEKPYKCPECGKSFSTKNSLTEHQRTHTGEKPYKCPECGKSFSTSGNLTEHQRTHTGEKPYKCPECGKSFSRSDKLTEHQRTHTGEKPYKCPECGKSFSDPGHLVRHQRTHTGEKPYKCPECGKSFSTKNSLTEHQRTHT

SEQ ID NO: 135 - protein, 140 aa, Artificial Sequence
  Synthetic construct
GEKPYKCPECGKSFSQSGHLTEHQRTHTGEKPYKCPECGKSFSRSDKLTEHQRTHTGEKPYKCPECGKSFSRSDKLTEHQRTHTGEKPYKCPECGKSFSRSDNLVRHQRTHTGEKPYKCPECGKSFSRSDKLTEHQRTHT

SEQ ID NO: 136 - protein, 168 aa, Artificial Sequence
  Synthetic construct
GEKPYKCPECGKSFSTTGALTEHQRTHTGEKPYKCPECGKSFSRNDALTEHQRTHTGEKPYKCPECGKSFSSRRTCRAHQRTHTGEKPYKCPECGKSFSSRRTCRAHQRTHTGEKPYKCPECGKSFSQSSSLVRHQRTHTGEKPYKCPECGKSFSTKNSLTEHQRTHT

SEQ ID NO: 137 - DNA, 18 bp, Homo sapiens
gaggttcccctgcggggc

SEQ ID NO: 138 - protein, 168 aa, Artificial Sequence
  Synthetic construct
GEKPYKCPECGKSFSQRAHLERHQRTHTGEKPYKCPECGKSFSRSDKLTEHQRTHTGEKPYKCPECGKSFSRNDALTEHQRTHTGEKPYKCPECGKSFSSRRTCRAHQRTHTGEKPYKCPECGKSFSTSGSLVRHQRTHTGEKPYKCPECGKSFSTSGNLVRHQRTHT

SEQ ID NO: 139 - protein, 168 aa, Artificial Sequence
  Synthetic construct
GEKPYKCPECGKSFSDPGHLVRHQRTHTGEKPYKCPECGKSFSRSDKLTEHQRTHTGEKPYKCPECGKSFSRNDALTEHQRTHTGEKPYKCPECGKSFSSKKHLAEHQRTHTGEKPYKCPECGKSFSTSGSLVRHQRTHTGEKPYKCPECGKSFSRSDNLVRHQRTHT

SEQ ID NO: 140 - protein, 168 aa, Artificial Sequence
  Synthetic construct
GEKPYKCPECGKSFSTSGHLVRHQRTHTGEKPYKCPECGKSFSRSDKLTEHQRTHTGEKPYKCPECGKSFSTKNSLTEHQRTHTGEKPYKCPECGKSFSSRRTCRAHQRTHTGEKPYKCPECGKSFSTSGSLVRHQRTHTGEKPYKCPECGKSFSTSGNLVRHQRTHT

SEQ ID NO: 141 - protein, 168 aa, Artificial Sequence
  Synthetic construct
GEKPYKCPECGKSFSQRAHLERHQRTHTGEKPYKCPECGKSFSRSDKLTEHQRTHTGEKPYKCPECGKSFSTTGALTEHQRTHTGEKPYKCPECGKSFSSRRTCRAHQRTHTGEKPYKCPECGKSFSTSGSLVRHQRTHTGEKPYKCPECGKSFSRSDNLVRHQRTHT

SEQ ID NO: 142 - protein, 168 aa, Artificial Sequence
  Synthetic construct
GEKPYKCPECGKSFSTSGHLVRHQRTHTGEKPYKCPECGKSFSRSDKLTEHQRTHTGEKPYKCPECGKSFSRNDALTEHQRTHTGEKPYKCPECGKSFSSRRTCRAHQRTHTGEKPYKCPECGKSFSQSSSLVRHQRTHTGEKPYKCPECGKSFSRSDNLVRHQRTHT

SEQ ID NO: 143 - protein, 168 aa, Artificial Sequence
  Synthetic construct
GEKPYKCPECGKSFSQRAHLERHQRTHTGEKPYKCPECGKSFSRSDKLTEHQRTHTGEKPYKCPECGKSFSRNDALTEHQRTHTGEKPYKCPECGKSFSSRRTCRAHQRTHTGEKPYKCPECGKSFSQSSSLVRHQRTHTGEKPYKCPECGKSFSTSGNLVRHQRTHT

SEQ ID NO: 144 - DNA, 18 bp, Homo sapiens
caccaagcccaggcagag

SEQ ID NO: 145 - protein, 168 aa, Artificial Sequence
  Synthetic construct
GEKPYKCPECGKSFSRSDNLVRHQRTHTGEKPYKCPECGKSFSQSSSLVRHQRTHTGEKPYKCPECGKSFSRADNLTEHQRTHTGEKPYKCPECGKSFSTSGNLVRHQRTHTGEKPYKCPECGKSFSQSGNLTEHQRTHTGEKPYKCPECGKSFSTKNSLTEHQRTHT

SEQ ID NO: 146 - protein, 168 aa, Artificial Sequence
  Synthetic construct
GEKPYKCPECGKSFSRSDNLVRHQRTHTGEKPYKCPECGKSFSQSGDLRRHQRTHTGEKPYKCPECGKSFSRADNLTEHQRTHTGEKPYKCPECGKSFSQSSSLVRHQRTHTGEKPYKCPECGKSFSTKNSLTEHQRTHTGEKPYKCPECGKSFSRADNLTEHQRTHT

SEQ ID NO: 147 - protein, 168 aa, Artificial Sequence
  Synthetic construct
GEKPYKCPECGKSFSRSDNLVRHQRTHTGEKPYKCPECGKSFSQSSSLVRHQRTHTGEKPYKCPECGKSFSRADNLTEHQRTHTGEKPYKCPECGKSFSQSSSLVRHQRTHTGEKPYKCPECGKSFSQSGNLTEHQRTHTGEKPYKCPECGKSFSTTGALTEHQRTHT

SEQ ID NO: 148 - protein, 168 aa, Artificial Sequence
  Synthetic construct
GEKPYKCPECGKSFSRSDNLVRHQRTHTGEKPYKCPECGKSFSQSGDLRRHQRTHTGEKPYKCPECGKSFSRNDALTEHQRTHTGEKPYKCPECGKSFSQSSSLVRHQRTHTGEKPYKCPECGKSFSQSGNLTEHQRTHTGEKPYKCPECGKSFSRNDALTEHQRTHT

SEQ ID NO: 149 - protein, 168 aa, Artificial Sequence
  Synthetic construct
GEKPYKCPECGKSFSRSDNLVRHQRTHTGEKPYKCPECGKSFSQSGDLRRHQRTHTGEKPYKCPECGKSFSRADNLTEHQRTHTGEKPYKCPECGKSFSTSGNLVRHQRTHTGEKPYKCPECGKSFSTKNSLTEHQRTHTGEKPYKCPECGKSFSSRRTCRAHQRTHT

SEQ ID NO: 150 - protein, 168 aa, Artificial Sequence
  Synthetic construct
GEKPYKCPECGKSFSRSDNLVRHQRTHTGEKPYKCPECGKSFSQSGDLRRHQRTHTGEKPYKCPECGKSFSRADNLTEHQRTHTGEKPYKCPECGKSFSTSGSLVRHQRTHTGEKPYKCPECGKSFSQSGNLTEHQRTHTGEKPYKCPECGKSFSQSGNLTEHQRTHT

SEQ ID NO: 151 - protein, 168 aa, Artificial Sequence
  Synthetic construct
GEKPYKCPECGKSFSRSDNLVRHQRTHTGEKPYKCPECGKSFSQSGDLRRHQRTHTGEKPYKCPECGKSFSRADNLTEHQRTHTGEKPYKCPECGKSFSTSGSLVRHQRTHTGEKPYKCPECGKSFSTKNSLTEHQRTHTGEKPYKCPECGKSFSSRRTCRAHQRTHT

SEQ ID NO: 152 - protein, 168 aa, Artificial Sequence
  Synthetic construct
GEKPYKCPECGKSFSRSDKLVRHQRTHTGEKPYKCPECGKSFSQSGDLRRHQRTHTGEKPYKCPECGKSFSRADNLTEHQRTHTGEKPYKCPECGKSFSTSGSLVRHQRTHTGEKPYKCPECGKSFSQSGNLTEHQRTHTGEKPYKCPECGKSFSSRRTCRAHQRTHT

SEQ ID NO: 153 - protein, 168 aa, Artificial Sequence
  Synthetic construct
GEKPYKCPECGKSFSRSDNLVRHQRTHTGEKPYKCPECGKSFSQSSSLVRHQRTHTGEKPYKCPECGKSFSRNDALTEHQRTHTGEKPYKCPECGKSFSTSGELVRHQRTHTGEKPYKCPECGKSFSQSGNLTEHQRTHTGEKPYKCPECGKSFSSRRTCRAHQRTHT

SEQ ID NO: 154 - DNA, 18 bp, Homo sapiens
gctgtggggcggctcgag

SEQ ID NO: 155 - protein, 168 aa, Artificial Sequence
  Synthetic construct
GEKPYKCPECGKSFSDPGHLVRHQRTHTGEKPYKCPECGKSFSTTGALTEHQRTHTGEKPYKCPECGKSFSSRRTCRAHQRTHTGEKPYKCPECGKSFSRSDNLVRHQRTHTGEKPYKCPECGKSFSQSSNLVRHQRTHTGEKPYKCPECGKSFSTSGNLVRHQRTHT

SEQ ID NO: 156 - protein, 168 aa, Artificial Sequence
  Synthetic construct
GEKPYKCPECGKSFSQRAHLERHQRTHTGEKPYKCPECGKSFSQSGHLTEHQRTHTGEKPYKCPECGKSFSTSGNLTEHQRTHTGEKPYKCPECGKSFSTSGSLVRHQRTHTGEKPYKCPECGKSFSRSDNLVRHQRTHTGEKPYKCPECGKSFSDPGHLVRHQRTHT

SEQ ID NO: 157 - protein, 168 aa, Artificial Sequence
  Synthetic construct
GEKPYKCPECGKSFSDPGHLVRHQRTHTGEKPYKCPECGKSFSTTGALTEHQRTHTGEKPYKCPECGKSFSSKKHLAEHQRTHTGEKPYKCPECGKSFSTSGELVRHQRTHTGEKPYKCPECGKSFSDCRDLARHQRTHTGEKPYKCPECGKSFSRSDELVRHQRTHT

SEQ ID NO: 158 - protein, 168 aa, Artificial Sequence
  Synthetic construct
GEKPYKCPECGKSFSTSGNLVRHQRTHTGEKPYKCPECGKSFSRSDKLTEHQRTHTGEKPYKCPECGKSFSTSGNLTEHQRTHTGEKPYKCPECGKSFSTSGELVRHQRTHTGEKPYKCPECGKSFSQSSSLVRHQRTHTGEKPYKCPECGKSFSDPGHLVRHQRTHT

SEQ ID NO: 159 - protein, 168 aa, Artificial Sequence
  Synthetic construct
GEKPYKCPECGKSFSDCRDLARHQRTHTGEKPYKCPECGKSFSRADNLTEHQRTHTGEKPYKCPECGKSFSSRRTCRAHQRTHTGEKPYKCPECGKSFSRSDNLVRHQRTHTGEKPYKCPECGKSFSQSSNLVRHQRTHTGEKPYKCPECGKSFSTSGSLVRHQRTHT

SEQ ID NO: 160 - protein, 168 aa, Artificial Sequence
  Synthetic construct
GEKPYKCPECGKSFSDPGHLVRHQRTHTGEKPYKCPECGKSFSTKNSLTEHQRTHTGEKPYKCPECGKSFSTSHSLTEHQRTHTGEKPYKCPECGKSFSQSSNLVRHQRTHTGEKPYKCPECGKSFSRSDNLVRHQRTHTGEKPYKCPECGKSFSDCRDLARHQRTHT

SEQ ID NO: 161 - protein, 168 aa, Artificial Sequence
  Synthetic construct
GEKPYKCPECGKSFSTSGNLVRHQRTHTGEKPYKCPECGKSFSRSDKLTEHQRTHTGEKPYKCPECGKSFSRSDKLTEHQRTHTGEKPYKCPECGKSFSDCRDLARHQRTHTGEKPYKCPECGKSFSQSGDLRRHQRTHTGEKPYKCPECGKSFSDCRDLARHQRTHT

SEQ ID NO: 162 - protein, 168 aa, Artificial Sequence
  Synthetic construct
GEKPYKCPECGKSFSQSSSLVRHQRTHTGEKPYKCPECGKSFSRNDALTEHQRTHTGEKPYKCPECGKSFSTSHSLTEHQRTHTGEKPYKCPECGKSFSRSDNLVRHQRTHTGEKPYKCPECGKSFSRSDNLVRHQRTHTGEKPYKCPECGKSFSTSGNLVRHQRTHT

SEQ ID NO: 163 - protein, 168 aa, Artificial Sequence
  Synthetic construct
GEKPYKCPECGKSFSDPGHLVRHQRTHTGEKPYKCPECGKSFSTSGNLTEHQRTHTGEKPYKCPECGKSFSTTGALTEHQRTHTGEKPYKCPECGKSFSQSGDLRRHQRTHTGEKPYKCPECGKSFSRSDELVRHQRTHTGEKPYKCPECGKSFSRSDNLVRHQRTHT

SEQ ID NO: 164 - protein, 168 aa, Artificial Sequence
  Synthetic construct
GEKPYKCPECGKSFSDCRDLARHQRTHTGEKPYKCPECGKSFSQNSTLTEHQRTHTGEKPYKCPECGKSFSTSGNLTEHQRTHTGEKPYKCPECGKSFSTSGSLVRHQRTHTGEKPYKCPECGKSFSRSDNLVRHQRTHTGEKPYKCPECGKSFSTSGNLVRHQRTHT

SEQ ID NO: 165 - DNA, 18 bp, Homo sapiens
atggccttcctctcctgc

SEQ ID NO: 166 - protein, 168 aa, Artificial Sequence
  Synthetic construct
GEKPYKCPECGKSFSQNSTLTEHQRTHTGEKPYKCPECGKSFSDPGHLVRHQRTHTGEKPYKCPECGKSFSRSDNLVRHQRTHTGEKPYKCPECGKSFSQRAHLERHQRTHTGEKPYKCPECGKSFSQSGDLRRHQRTHTGEKPYKCPECGKSFSDCRDLARHQRTHT

SEQ ID NO: 167 - protein, 168 aa, Artificial Sequence
  Synthetic construct
GEKPYKCPECGKSFSSKKHLAEHQRTHTGEKPYKCPECGKSFSTSGNLVRHQRTHTGEKPYKCPECGKSFSRSDELVRHQRTHTGEKPYKCPECGKSFSQSGDLRRHQRTHTGEKPYKCPECGKSFSQRAHLERHQRTHTGEKPYKCPECGKSFSRSDELVRHQRTHT

SEQ ID NO: 168 - protein, 168 aa, Artificial Sequence
  Synthetic construct
GEKPYKCPECGKSFSRSDKLTEHQRTHTGEKPYKCPECGKSFSQRAHLERHQRTHTGEKPYKCPECGKSFSRSDNLVRHQRTHTGEKPYKCPECGKSFSQRAHLERHQRTHTGEKPYKCPECGKSFSDPGALVRHQRTHTGEKPYKCPECGKSFSDCRDLARHQRTHT

SEQ ID NO: 169 - protein, 168 aa, Artificial Sequence
  Synthetic construct
GEKPYKCPECGKSFSSKKHLAEHQRTHTGEKPYKCPECGKSFSTSGNLVRHQRTHTGEKPYKCPECGKSFSDPGHLVRHQRTHTGEKPYKCPECGKSFSQSSNLVRHQRTHTGEKPYKCPECGKSFSRSDKLVRHQRTHTGEKPYKCPECGKSFSDPGHLVRHQRTHT

SEQ ID NO: 170 - protein, 168 aa, Artificial Sequence
  Synthetic construct
GEKPYKCPECGKSFSSKKHLAEHQRTHTGEKPYKCPECGKSFSDPGHLVRHQRTHTGEKPYKCPECGKSFSQRAHLERHQRTHTGEKPYKCPECGKSFSRSDNLVRHQRTHTGEKPYKCPECGKSFSDPGHLVRHQRTHTGEKPYKCPECGKSFSQSGDLRRHQRTHT

SEQ ID NO: 171 - protein, 168 aa, Artificial Sequence
  Synthetic construct
GEKPYKCPECGKSFSTSGNLTEHQRTHTGEKPYKCPECGKSFSDPGHLVRHQRTHTGEKPYKCPECGKSFSQSGDLRRHQRTHTGEKPYKCPECGKSFSRSDNLVRHQRTHTGEKPYKCPECGKSFSDPGHLVRHQRTHTGEKPYKCPECGKSFSQSGDLRRHQRTHT

SEQ ID NO: 172 - protein, 168 aa, Artificial Sequence
  Synthetic construct
GEKPYKCPECGKSFSRNDALTEHQRTHTGEKPYKCPECGKSFSDPGHLVRHQRTHTGEKPYKCPECGKSFSQRAHLERHQRTHTGEKPYKCPECGKSFSRSDNLVRHQRTHTGEKPYKCPECGKSFSDPGHLVRHQRTHTGEKPYKCPECGKSFSTSGSLVRHQRTHT

SEQ ID NO: 173 - protein, 168 aa, Artificial Sequence
  Synthetic construct
GEKPYKCPECGKSFSSRRTCRAHQRTHTGEKPYKCPECGKSFSTSGNLVRHQRTHTGEKPYKCPECGKSFSRSDNLVRHQRTHTGEKPYKCPECGKSFSTSGHLVRHQRTHTGEKPYKCPECGKSFSQSGDLRRHQRTHTGEKPYKCPECGKSFSDCRDLARHQRTHT

SEQ ID NO: 174 - DNA, 18 bp, Homo sapiens
gcccagttgggcaccatc

SEQ ID NO: 175 - protein, 168 aa, Artificial Sequence
  Synthetic construct
GEKPYKCPECGKSFSDPGHLVRHQRTHTGEKPYKCPECGKSFSRNDALTEHQRTHTGEKPYKCPECGKSFSQSGNLTEHQRTHTGEKPYKCPECGKSFSTSGSLVRHQRTHTGEKPYKCPECGKSFSTSGHLVRHQRTHTGEKPYKCPECGKSFSTSGNLVRHQRTHT

SEQ ID NO: 176 - protein, 168 aa, Artificial Sequence
  Synthetic construct
GEKPYKCPECGKSFSQSSSLVRHQRTHTGEKPYKCPECGKSFSRNDALTEHQRTHTGEKPYKCPECGKSFSQSGNLTEHQRTHTGEKPYKCPECGKSFSTSGSLVRHQRTHTGEKPYKCPECGKSFSTSGHLVRHQRTHTGEKPYKCPECGKSFSTSGNLVRHQRTHT

SEQ ID NO: 177 - protein, 168 aa, Artificial Sequence
  Synthetic construct
GEKPYKCPECGKSFSQSSSLVRHQRTHTGEKPYKCPECGKSFSRNDALTEHQRTHTGEKPYKCPECGKSFSQSGHLTEHQRTHTGEKPYKCPECGKSFSTSGSLVRHQRTHTGEKPYKCPECGKSFSTSGHLVRHQRTHTGEKPYKCPECGKSFSTSGNLVRHQRTHT

SEQ ID NO: 178 - protein, 168 aa, Artificial Sequence
  Synthetic construct
GEKPYKCPECGKSFSTSGSLVRHQRTHTGEKPYKCPECGKSFSRNDALTEHQRTHTGEKPYKCPECGKSFSQSGHLTEHQRTHTGEKPYKCPECGKSFSTSGSLVRHQRTHTGEKPYKCPECGKSFSTSGHLVRHQRTHTGEKPYKCPECGKSFSTSGNLVRHQRTHT

SEQ ID NO: 179 - protein, 168 aa, Artificial Sequence
  Synthetic construct
GEKPYKCPECGKSFSTSGHLVRHQRTHTGEKPYKCPECGKSFSRNDALTEHQRTHTGEKPYKCPECGKSFSQSGHLTEHQRTHTGEKPYKCPECGKSFSTSGSLVRHQRTHTGEKPYKCPECGKSFSQRAHLERHQRTHTGEKPYKCPECGKSFSTSGNLVRHQRTHT

SEQ ID NO: 180 - protein, 168 aa, Artificial Sequence
  Synthetic construct
GEKPYKCPECGKSFSTSGHLVRHQRTHTGEKPYKCPECGKSFSRNDALTEHQRTHTGEKPYKCPECGKSFSQSGNLTEHQRTHTGEKPYKCPECGKSFSQSSNLVRHQRTHTGEKPYKCPECGKSFSTSGHLVRHQRTHTGEKPYKCPECGKSFSTSGNLVRHQRTHT

SEQ ID NO: 181 - protein, 168 aa, Artificial Sequence
  Synthetic construct
GEKPYKCPECGKSFSQRAHLERHQRTHTGEKPYKCPECGKSFSRNDALTEHQRTHTGEKPYKCPECGKSFSQSGHLTEHQRTHTGEKPYKCPECGKSFSQSSSLVRHQRTHTGEKPYKCPECGKSFSTSGHLVRHQRTHTGEKPYKCPECGKSFSTSGNLVRHQRTHT

SEQ ID NO: 182 - DNA, 18 bp, Homo sapiens
gtccatgaagaagatggc

SEQ ID NO: 183 - protein, 168 aa, Artificial Sequence
  Synthetic construct
GEKPYKCPECGKSFSTSGHLVRHQRTHTGEKPYKCPECGKSFSTSGSLVRHQRTHTGEKPYKCPECGKSFSDCRDLARHQRTHTGEKPYKCPECGKSFSTSGNLVRHQRTHTGEKPYKCPECGKSFSRSDKLTEHQRTHTGEKPYKCPECGKSFSQSSSLVRHQRTHT

SEQ ID NO: 184 - protein, 168 aa, Artificial Sequence
  Synthetic construct
GEKPYKCPECGKSFSTSGELVRHQRTHTGEKPYKCPECGKSFSRSDELVRHQRTHTGEKPYKCPECGKSFSRSDDLVRHQRTHTGEKPYKCPECGKSFSTSGNLVRHQRTHTGEKPYKCPECGKSFSRSDKLTEHQRTHTGEKPYKCPECGKSFSQSSSLVRHQRTHT

SEQ ID NO: 185 - protein, 168 aa, Artificial Sequence
  Synthetic construct
GEKPYKCPECGKSFSRSDELVRHQRTHTGEKPYKCPECGKSFSQSSSLVRHQRTHTGEKPYKCPECGKSFSDCRDLARHQRTHTGEKPYKCPECGKSFSTSGNLVRHQRTHTGEKPYKCPECGKSFSRSDKLTEHQRTHTGEKPYKCPECGKSFSQSSSLVRHQRTHT

SEQ ID NO: 186 - protein, 168 aa, Artificial Sequence
  Synthetic construct
GEKPYKCPECGKSFSDPGNLVRHQRTHTGEKPYKCPECGKSFSRSDKLVRHQRTHTGEKPYKCPECGKSFSDCRDLARHQRTHTGEKPYKCPECGKSFSTSGNLVRHQRTHTGEKPYKCPECGKSFSRSDKLTEHQRTHTGEKPYKCPECGKSFSQSSSLVRHQRTHT

SEQ ID NO: 187 - protein, 168 aa, Artificial Sequence
  Synthetic construct
GEKPYKCPECGKSFSRSDDLVRHQRTHTGEKPYKCPECGKSFSTSGNLVRHQRTHTGEKPYKCPECGKSFSRSDKLVRHQRTHTGEKPYKCPECGKSFSQSSSLVRHQRTHTGEKPYKCPECGKSFSTTGALTEHQRTHTGEKPYKCPECGKSFSQSSSLVRHQRTHT

SEQ ID NO: 188 - protein, 168 aa, Artificial Sequence
  Synthetic construct
GEKPYKCPECGKSFSDPGNLVRHQRTHTGEKPYKCPECGKSFSRSDELVRHQRTHTGEKPYKCPECGKSFSDCRDLARHQRTHTGEKPYKCPECGKSFSTSGNLVRHQRTHTGEKPYKCPECGKSFSRSDKLTEHQRTHTGEKPYKCPECGKSFSQSSSLVRHQRTHT

SEQ ID NO: 189 - protein, 168 aa, Artificial Sequence
  Synthetic construct
GEKPYKCPECGKSFSQSGDLRRHQRTHTGEKPYKCPECGKSFSRSDELVRHQRTHTGEKPYKCPECGKSFSDCRDLARHQRTHTGEKPYKCPECGKSFSTSGNLVRHQRTHTGEKPYKCPECGKSFSRADNLTEHQRTHTGEKPYKCPECGKSFSQSSSLVRHQRTHT

SEQ ID NO: 190 - protein, 168 aa, Artificial Sequence
  Synthetic construct
GEKPYKCPECGKSFSDPGNLVRHQRTHTGEKPYKCPECGKSFSQSSNLVRHQRTHTGEKPYKCPECGKSFSDCRDLARHQRTHTGEKPYKCPECGKSFSTSGNLVRHQRTHTGEKPYKCPECGKSFSRSDKLTEHQRTHTGEKPYKCPECGKSFSQSSSLVRHQRTHT

SEQ ID NO: 191 - protein, 168 aa, Artificial Sequence
  Synthetic construct
GEKPYKCPECGKSFSQSGDLRRHQRTHTGEKPYKCPECGKSFSRSDELVRHQRTHTGEKPYKCPECGKSFSQSGDLRRHQRTHTGEKPYKCPECGKSFSTSGNLVRHQRTHTGEKPYKCPECGKSFSRSDKLTEHQRTHTGEKPYKCPECGKSFSRSDKLVRHQRTHT

SEQ ID NO: 192 - protein, 12 aa, Artificial sequence
  Synthetic construct
PVRRPRRRRRRK

SEQ ID NO: 193 - protein, 12 aa, Artificial sequence
  Synthetic construct
THRLPRRRRRRK

SEQ ID NO: 194 - protein, 9 aa, Artificial sequence
  Synthetic construct
RRRRRRRRR

SEQ ID NO: 195 - protein, 9 aa, Influenza A virus
YPYDVPDYA

SEQ ID NO: 196 - protein, 7 aa, Artificial Sequence
  Synthetic construct
PKKKRKV

SEQ ID NO: 197 - protein, 7 aa, Artificial Sequence
  Synthetic construct
YKDDDDK

SEQ ID NO: 198 - DNA, 57 bp, Artificial sequence
  Synthetic construct
tcgacaggcccaggcggccctcgaggatatcatgatgactagtggccaggccggccc

SEQ ID NO: 199 - DNA, 57 bp, Artificial sequence
  Synthetic construct
aattgggccggcctggccactagtcatcatgatatcctcgagggccgcctgggcctg

SEQ ID NO: 200 - DNA, 23 bp, Artificial Sequence
  Synthetic construct
cgccgcatgcattcatgcaggcc

SEQ ID NO: 201 - DNA, 17 bp, Artificial Sequence
  Synthetic construct
tgcatgaatgcatgcgg

SEQ ID NO: 202 - DNA, 37 bp, Artificial sequence
  Synthetic construct
aatcgcgagctccttaagaaactggcagcttccactt

SEQ ID NO: 203 - DNA, 31 bp, Artificial sequence
  Synthetic construct
aatcgcctcgagctgccgggtccgcgcggcg

SEQ ID NO: 204 - DNA, 36 bp, Artificial Sequence
  Synthetic construct
gctagctgtcgacacatggtgcgtgataacttgccc

SEQ ID NO: 205 - DNA, 41 bp, Artificial Sequence
  Synthetic construct
gctagctggtaccaggcctgctgctacctgctccagaaggc

SEQ ID NO: 206 - DNA, 40 bp, Artificial Sequence
  Synthetic construct
gctagctgtcgacataagccagtgacaaaaagatacatac

SEQ ID NO: 207 - DNA, 44 bp, Artificial Sequence
  Synthetic contruct
gctagctggtaccaggccttatttgatctctgtggcttcttgag

SEQ ID NO: 208 - DNA, 37 bp, Artificial Sequence
  Synthetic construct
ctagctgatatcagcttagcggtttacatgacttgac

SEQ ID NO: 209 - DNA, 34 bp, Artificial Sequence
  Synthetic construct
ctagctaagcttcacgcaggagaggaaggccatg

SEQ ID NO: 210 - DNA, 42 bp, Artificial Sequence
  Synthetic construct
gctagctgtcgacttaaattcctatttattaacctttttagc

SEQ ID NO: 211 - DNA, 42 bp, Artificial Sequence
  Synthetic construct
gctagctggtaccaggcctgtcaccacccacagtaaaggttc

SEQ ID NO: 212 - DNA, 37 bp, Artificial Sequence
  Synthetic construct
ctagctgatatcgctagccatgctcctgaatatgtat

SEQ ID NO: 213 - DNA, 43 bp, Artificial Sequence
  Synthetic construct
ctagctaagcttggcaggagccctcttcttcatggactcctgg

SEQ ID NO: 214 - DNA, 72 bp, Artificial sequence
  Synthetic construct
agcttgtgaactgtcttggaagtggatcctccagcccctgctacatggagcaaaaacgagctgtcccatggc

SEQ ID NO: 215 - DNA, 72 bp, Artificial sequence
  Synthetic construct
tcgagccatgggacagctcgtttttgctccatgtagcaggggctggaggatccacttccaagacagttcaca

SEQ ID NO: 216 - DNA, 72 bp, Artificial sequence
  Synthetic construct
agcttaggcagtggcctttgtccctcatctcctctcccacccccaatttaggataaagtatctgcccatggc

SEQ ID NO: 217 - DNA, 72 bp, Artificial sequence
  Synthetic construct
tcgagccatgggcagatactttatcctaaattgggggtgggagaggagatgagggacaaaggccactgccta

SEQ ID NO: 218 - DNA, 72 bp, Artificial sequence
  Synthetic construct
agcttagacgttgagacccactttctgtaaggtcggcttcttcattgtttgaatttcttgaggttccatggc

SEQ ID NO: 219 - DNA, 72 bp, Artificial sequence
  Synthetic construct
tcgagccatggaacctcaagaaattcaaacaatgaagaagccgaccttacagaaagtgggtctcaacgtcta

SEQ ID NO: 220 - DNA, 72 bp, Artificial sequence
  Synthetic construct
agcttattgtttgaatttcttgaggtttcacggagccacgcgctggaaccttccatagtctctccccatggc

SEQ ID NO: 221 - DNA, 72 bp, Artificial sequence
  Synthetic construct
tcgagccatggggagagactatggaaggttccagcgcgtggctccgtgaaacctcaagaaattcaaacaata

SEQ ID NO: 222 - DNA, 72 bp, Artificial sequence
  Synthetic construct
agcttaaaaaagactcctgcccttcagggcctggaagggggcggcagctttgtgctttttagtggccatggc

SEQ ID NO: 223 - DNA, 72 bp, Artificial sequence
  Synthetic construct
tcgagccatggccactaaaaagcacaaagctgccgcccccttccaggccctgaagggcaggagtctttttta

SEQ ID NO: 224 - DNA, 72 bp, Artificial sequence
  Synthetic construct
agcttgcgtgctccctcttaagtttagaggccgttgaggagccgaagtggacagcagtttactggccatggc

SEQ ID NO: 225 - DNA, 72 bp, Artificial sequence
  Synthetic construct
tcgagccatggccagtaaactgctgtccacttcggctcctcaacggcctctaaacttaagagggagcacgca

SEQ ID NO: 226 - DNA, 72 bp, Artificial sequence
  Synthetic construct
agcttggcagggaagacggagaagaaaccacccgtgggccctggctctgtgtccagttgttccgtccatggc

SEQ ID NO: 227 - DNA, 72 bp, Artificial sequence
  Synthetic construct
tcgagccatggacggaacaactggacacagagccagggcccacgggtggtttcttctccgtcttccctgcca

SEQ ID NO: 228 - DNA, 72 bp, Artificial sequence
  Synthetic construct
agcttgtctgtcaaactctaccctctctcctccacatcccccaccttttctttcaggaaggaaatccatggc

SEQ ID NO: 229 - DNA, 72 bp, Artificial sequence
  Synthetic construct
tcgagccatggatttccttcctgaaagaaaaggtgggggatgtggaggagagagggtagagtttgacagaca

SEQ ID NO: 230 - DNA, 72 bp, Artificial sequence
  Synthetic construct
agcttagtggaaggtctggagctttgggaggagacggggaggacagactggaggcgtgttcctccccatggc

SEQ ID NO: 231 - DNA, 72 bp, Artificial sequence
  Synthetic construct
tcgagccatggggaggaacacgcctccagtctgtcctccccgtctcctcccaaagctccagaccttccacta

SEQ ID NO: 232 - DNA, 72 bp, Artificial Sequence
  Synthetic construct
agcttggacgaggactgcccccctccctcgggcaactactactgatgctgtccaggcatcgcccaccatggc

SEQ ID NO: 233 - DNA, 72 bp, Artificial Sequence
  Synthetic construct
tcgagccatggtgggcgatgcctggacagcatcagtagtagttgcccgagggaggggggcagtcctcgtcca

SEQ ID NO: 234 - DNA, 72 bp, Artificial Sequence
  Synthetic construct
agcttcgagttcaatcgcggggtatagaggttcccctgcggggcaaaatgcagagcttgacacaaccatggc

SEQ ID NO: 235 - DNA, 72 bp, Artificial Sequence
  Synthetic construct
tcgagccatggttgtgtcaagctctgcattttgccccgcaggggaacctctataccccgcgattgaactcga

SEQ ID NO: 236 - DNA, 72 bp, Artificial Sequence
  Synthetic construct
agcttacctgattgttttcctaaattcaccaagcccaggcagaggtcagatgactaattgggataccatggc

SEQ ID NO: 237 - DNA, 72 bp, Artificial Sequence
  Synthetic construct
tcgagccatggtatcccaattagtcatctgacctctgcctgggcttggtgaatttaggaaaacaatcaggta

SEQ ID NO: 238 - DNA, 72 bp, Artificial Sequence
  Synthetic construct
agcttactgctttgaatacaccaattgctgtggggcggctcgaggaagagaagacaccagtgcctccatggc

SEQ ID NO: 239 - DNA, 72 bp, Artificial Sequence
  Synthetic construct
tcgagccatggaggcactggtgtcttctcttcctcgagccgccccacagcaattggtgtattcaaagcagta

SEQ ID NO: 240 - DNA, 72 bp, Artificial Sequence
  Synthetic construct
agcttgctgggactctgatcccagccatggccttcctctcctgcgtgagaccagaaagctgggagccatggc

SEQ ID NO: 241 - DNA, 72 bp, Artificial Sequence
  Synthetic construct
tcgagccatggctcccagctttctggtctcacgcaggagaggaaggccatggctgggatcagagtcccagca

SEQ ID NO: 242 - DNA, 72 bp, Artificial Sequence
  Synthetic construct
agctttaagtgggtaaatattaaattgcccagttgggcaccatcctgaatattatctctaaagaaccatggc

SEQ ID NO: 243 - DNA, 72 bp, Artificial Sequence
  Synthetic construct
tcgagccatggttctttagagataatattcaggatggtgcccaactgggcaatttaatatttacccacttaa

SEQ ID NO: 244 - DNA, 72 bp, Artificial Sequence
  Synthetic construct
agcttccagcacagtaagcaccaggagtccatgaagaagatggctcctgccatggaatcccctacccatggc

SEQ ID NO: 245 - DNA, 72 bp, Artificial Sequence
  Synthetic construct
tcgagccatgggtaggggattccatggcaggagccatcttcttcatggactcctggtgcttactgtgctgga

SEQ ID NO: 246 - DNA, 61 bp, Artificial sequence
  Synthetic construct
aatcgctctagagatatcatatatctcgagatatataccggtgagcagaaactcatctctg

SEQ ID NO: 247 - DNA, 40 bp, Artificial sequence
  Synthetic construct
gcgattgaattcgcggccgcttacagatcttcctcagaga

SEQ ID NO: 248 - DNA, 31 bp, Artificial sequence
  Synthetic construct
aatcgcgatatcatggatgctaagtccctga

SEQ ID NO: 249 - DNA, 32 bp, Artificial sequence
  Synthetic construct
gcgattctcgagccccactttacgtttctttt

SEQ ID NO: 250 - DNA, 36 bp, Artificial sequence
  Synthetic construct
aatcgcctcgagcccgggccgggtgaaaagccctat

SEQ ID NO: 251 - DNA, 29 bp, Artificial sequence
  Synthetic construct
gcgattaccggtctgtgctgatgagcccc

SEQ ID NO: 252 - DNA, 36 bp, Artificial sequence
  Synthetic construct
aatcgcctcgagcccgggccaggcgaaaagccctac

SEQ ID NO: 253 - DNA, 30 bp, Artificial sequence
  Synthetic construct
gcgattaccggtctgtgctgaactaccgcc

SEQ ID NO: 254 - DNA, 53 bp, Artificial sequence
  Synthetic construct
aatcgcgatatcatggcggcggcggttcggatgaacatccagatgctgctgga

SEQ ID NO: 255 - DNA, 54 bp, Artificial sequence
  Synthetic construct
atccagatgctgctggaggcggccgactatctggagcggcgggagagagaagct

SEQ ID NO: 256 - DNA, 47 bp, Artificial sequence
  Synthetic construct
ggtatggtaacatggaggcataaccatgttcagcttctctctcccgc

SEQ ID NO: 257 - DNA, 53 bp, Artificial sequence
  Synthetic construct
gcgattctcgagccccactttacgtttctttttcgggtatggtaacatggagg

SEQ ID NO: 258 - DNA, 48 bp, Artificial sequence
  Synthetic construct
gcgattaccggtgaattcatatatggatccgagcagaaactcatctct

SEQ ID NO: 259 - DNA, 40 bp, Artificial sequence
  Synthetic construct
gcgattaagcttgcggccgcttacagatcttcctcagaga

SEQ ID NO: 260 - DNA, 34 bp, Artificial sequence
  Synthetic construct
gcgattgatatcatgccgaaaaagaaacgtaaag

SEQ ID NO: 261 - DNA, 47 bp, Artificial sequence
  Synthetic construct
gcgattgaattcgctgccgccgctgccgccaccggtatgagtcctct

SEQ ID NO: 262 - DNA, 28 bp, Artificial sequence
  Synthetic construct
gcgattgaattccgcacactggttacct

SEQ ID NO: 263 - DNA, 28 bp, Artificial sequence
  Synthetic construct
gcgattggatccatagcccaggctaacc

SEQ ID NO: 264 - DNA, 90 bp, Artificial Sequence
  Synthetic construct
aattcgggcgcgccgacgcgctggacgatttcgatctcgacatgctgggttctgatgccctcgatgactttgacctggatatgttgggaa

SEQ ID NO: 265 - DNA, 91 bp, Artificial Sequence
  Synthetic construct
gtcgcttcccaacatatccaggtcaaagtcatcgagggcatcagaacccagcatgtcgagatcgaaatcgtccagcgcgtcggcgcgcccg

SEQ ID NO: 266 - DNA, 81 bp, Artificial Sequence
  Synthetic construct
gcgacgcattggatgactttgatctggacatgctcggctccgatgctctggacgatttcgatctcgatatgttaattaacg

SEQ ID NO: 267 - DNA, 80 bp, Artificial Sequence
  Synthetic construct
gatccgttaattaacatatcgagatcgaaatcgtccagagcatcggagccgagcatgtccagatcaaagtcatccaatgc